(19) United States
(12) Patent Application Publication
(10) Pub. No.: US 2023/0365585 A1
(43) Pub. Date: Nov. 16, 2023

(54) BENZODIAZEPINE DERIVATIVES USEFUL IN TREATING A RESPIRATORY SYNCYTIAL VIRUS INFECTION

(71) Applicant: REVIRAL LIMITED, Hertfordshire (GB)

(72) Inventors: Matthew BARRETT, Stevenage (GB); George Stuart COCKERILL, Stevenage (GB); James GOOD, Stevenage (GB); Craig Alex AVERY, Nottingham (GB); Edward James COCHRANE, Nottingham (GB); Stuart Thomas ONIONS, Nottingham (GB); Andrew Joseph WARNER, Nottingham (GB)

(21) Appl. No.: 18/004,496

(22) PCT Filed: Jul. 7, 2021
(86) PCT No.: PCT/GB2021/051732

§ 371 Date: Jan. 10, 2023

(30) Foreign Application Priority Data
Jul. 7, 2020 (GB) 2010408.9

(51) Int. Cl. C07D 498/04; C07D 513/04; A61K 45/06; C07D 498/18; A61P 31/14

(52) U.S. Cl. CPC C07D 498/04; A61K 45/06; A61P 31/14; C07D 498/18; C07D 513/04

(57) ABSTRACT

Benzodiazepine derivatives of formula (Ib) wherein: R¹ is H or halo; Y is selected from O, S, SO, SO₂ and NR; one or two of V, W and X is or are N or CH and the other one or two is or are CH; R² is a group selected from C₁-C₆ alkyl, C₁-C₆ hydroxyalkyl, C₁-C₆ haloalkyl, halo, —OR, —NHR″, —SOₘNR₂, —SOₘR, nitro, —CO₂R, —CN, —CONR₂, —NHCOR and —NR¹¹R¹²; each R is independently H or C₁-C₆ alkyl; R¹¹ and R¹² are each independently H or C₁-C₆ alkyl; or R¹¹ and R¹² form, together with the N atom to which they are attached, either (a) a morpholine ring which is optionally bridged by a —CH₂— group linking two ring carbon atoms that are positioned para to each other, or (b) a spiro group of the following formula (b): R″ is C₃-C₆ cycloalkyl; m is 1 or 2; n is 0, 1 or 2; and each of R³ to R¹⁰ is independently selected from H, C₁-C₆ alkyl, halo, —OR, -NR₂,—NHR″, —SOₘNR₂, —SOₘR, nitro, —CO₂R, —CN, —CONR₂, —NHCOR, —NR¹³R ⁴ whereinR¹³ and R¹⁴ form, together with the N atom to which they are attached, a morpholine ring, and the following options (i) to (iii): (i) any two of R³ to R¹⁰ that bond to the same carbon atom form a C₃-C₆ spiro ring; (ii) any two of R³ to R¹⁰ that bond to non-adjacent carbon atoms form a C₁-C₃ bridgehead group linking the carbon atoms to which they are bonded; and (iii) any two of R³ to R¹⁰ that bond to adjacent carbon atoms form, together with the carbon atoms to which they are bonded, a C₃-C₆ cycloalkyl group; and wherein each alkyl group or moiety recited above is linear or branched; and the pharmaceutically acceptable salts thereof are inhibitors of RSV and can therefore be used to treat or prevent an RSV infection.

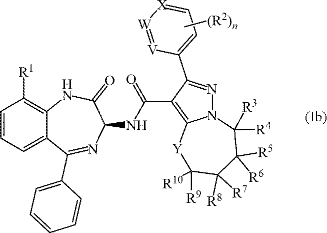

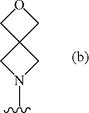

### FIELD OF THE INVENTION

The present invention relates to benzodiazepine derivatives and to their use in treating or preventing a respiratory syncytial virus (RSV) infection.

### BACKGROUND TO THE INVENTION

RSV is a negative-sense, single-stranded RNA virus of the Paramyxoviridae family. RSV is readily transmitted by secretions from an infected person via surfaces or hand-to-hand transfer. Unlike influenza, it is not transmitted by small-particle aerosols. Following successful inoculation, the incubation period is between four and six days during which time the virus spreads from the nasopharynx to the lower respiratory tract by fusion of infected with uninfected cells and by sloughing of the necrotic epithelium. In infants, coupled with increased mucus secretion and oedema, this can lead to mucus plugging causing hyper-inflation and collapse of distal lung tissue indicative of bronchiolitis. Hypoxia is common and the ability to feed is often impaired because of respiratory distress. In RSV pneumonia, inflammatory infiltration of the airways consists of mononuclear cells and is more generalised, with involvement of the bronchioles, bronchi and alveoli. The duration and degree of viral shedding has been found to correlate with the clinical signs and severity of disease.

RSV is the leading cause of serious respiratory tract infections in infants and young children throughout the world. The highest morbidity and mortality occurs in those born prematurely and for those with chronic lung or heart disease, although many infants hospitalised for RSV infection are otherwise healthy. Severe RSV infection in infancy can lead to several years of recurrent wheezing and is linked to the later development of asthma.

RSV is also a major cause of morbidity and mortality in the elderly and in immunocompromised children and adults as well as those with chronic obstructive pulmonary disease (COPD) and congestive heart failure (CHF).

RSV has a seasonal incidence; it is highly predictable and occurs in the winters of both hemispheres, from September to May in Europe and North America, peaking in December and January, and can occur throughout the year in tropical countries. It affects >90% of infants and young children by the age of two years and as natural immunity is short-lived; many will be re -infected each year. As with influenza, in elderly people, RSV causes around 10% of winter hospitalisations with an associated mortality of 10%.

Current anti-RSV treatment involves the use of a monoclonal antibody to RSV, called palivizumab. Such use of palivizumab is a prophylactic, rather than therapeutic, treatment of RSV. Although this antibody is often effective, its use is restricted to preterm infants and infants at high risk. Indeed, its limited utility means that it is unavailable for many people in need of anti-RSV treatment. There is therefore an urgent need for effective alternatives to existing anti-RSV treatment.

Small molecules have also been proposed as inhibitors of RSV. These include benzimidazoles and benzodiazepines. For instance, the discovery and initial development of RSV604, a benzodiazepine compound having sub-micromolar anti-RSV activity, is described in Antimicrobial Agents and Chemotherapy, September 2007, 3346-3353 (Chapman et al). Benzodiazepine inhibitors of RSV are also disclosed in publications including WO2004/026843 and WO2005/089770 (Arrow Therapeutics Limited); WO2016/166546 and WO2018/033714 (Durham University); and WO2017/015449, WO2018/129287 and WO2018/226801 (Enanta Pharmaceuticals, Inc.).

There exists a need to identify further compounds that have anti-RSV activity, in particular compounds having a combination of potent anti-viral activity and favourable pharmacokinetic properties.

### SUMMARY OF THE INVENTION

It has now been found that a novel series of benzodiazepine derivatives have potent anti-RSV activity with favourable pharmacokinetics and good physicochemical properties. Accordingly, the present invention provides a compound which is a benzodiazepine derivative of formula (Ib):

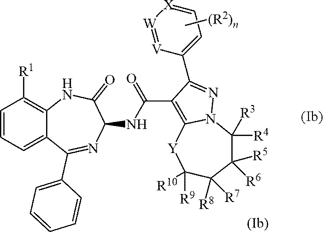

wherein:
R¹ is H or halo;
Y is selected from O, S, SO, SO₂ and NR;
one or two of V, W and X is or are N or CH and the other one or two is or are CH;
R² is a group selected from C₁-C₆ alkyl, C₁-C₆ hydroxyalkyl, C₁-C₆ haloalkyl, halo, —OR, —NHR″, —SOₘNR₂, —SOₘR, nitro, —CO₂R, —CN, —CONR₂, —NHCOR and —NR¹¹R¹²; each R is independently H or C₁-C₆ alkyl;
R¹¹ and R¹² are each independently H or C₁-C₆ alkyl; or R¹¹ and R¹² form, together with the N atom to which they are attached, either (a) a morpholine ring which is optionally bridged by a —CH₂— group linking two ring carbon atoms that are positioned para to each other, or (b) a spiro group of the following formula (b):

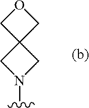

R″ is C₃-C₆ cycloalkyl;
m is 1 or 2;
n is 0, 1 or 2; and
each of R³ to R¹⁰ is independently selected from H, C₁-C₆ alkyl, halo, —OR, —NR₂, —NHR″, —SOₘNR₂, —SOₘR, nitro, —CO₂R, —CN, —CONR₂, —NHCOR, —NR¹³R¹⁴ wherein R¹³ and R¹⁴ form, together with the N atom to which they are attached, a morpholine ring, and the following options (i) to (iii):
   (i) any two of R³ to R¹⁰ that bond to the same carbon atom form a C₃-C₆ spiro ring;
   (ii) any two of R³ to R¹⁰ that bond to non-adjacent carbon atoms form a C₁-C₃ bridgehead group linking the carbon atoms to which they are bonded; and
   (iii) any two of R³ to R¹⁰ that bond to adjacent carbon atoms form, together with the carbon atoms to which they are bonded, a C₃-C₆ cycloalkyl group;
and wherein each alkyl group or moiety recited above is linear or branched;
or a pharmaceutically acceptable salt thereof.

In one embodiment, the present invention provides a compound which is a benzodiazepine derivative of formula (I):

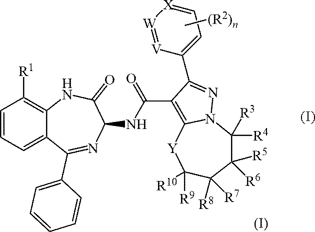

wherein:
R¹ is H or halo;
Y is selected from O, SO, SO₂ and NR;
one or two of V, W and X is or are N or CH and the other one or two is or are CH;
R² is a group selected from C₁-C₆ alkyl, C₁-C₆ hydroxyalkyl, C₁-C₆ haloalkyl, halo, —OR, —NHR″, —SOₘNR₂, —SOₘR, nitro, —CO₂R, —CN, —CONR₂, —NHCOR and —NR¹¹R¹²; each R is independently H or C₁-C₆ alkyl;
R¹¹ and R¹² are each independently H or C₁-C₆ alkyl; or R¹¹ and R¹² form, together with the N atom to which they are attached, either (a) a morpholine ring which is optionally bridged by a —CH₂— group linking two ring carbon atoms that are positioned para to each other, or (b) a spiro group of the following formula (b):

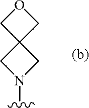

R″ is C₃-C₆ cycloalkyl;
m is 1 or 2;
n is 0, 1 or 2; and
each of R³ to R¹⁰ is independently selected from H, C₁-C₆ alkyl, halo, —OR, —NR₂, —NHR″, —SOₘNR₂, —SOₘR, nitro, —CO₂R, —CN, —CONR₂, —NHCOR and the following options (i) to (iii):
   (i) any two of R³ to R¹⁰ that bond to the same carbon atom form a C₃-C₆ spiro ring;
   (ii) any two of R³ to R¹⁰ that bond to non-adjacent carbon atoms form a C₁-C₃ bridgehead group linking the carbon atoms to which they are bonded; and
   (iii) any two of R³ to R¹⁰ that bond to adjacent carbon atoms form, together with the carbon atoms to which they are bonded, a C₃-C₆ cycloalkyl group;
and wherein each alkyl group or moiety recited above is linear or branched;
or a pharmaceutically acceptable salt thereof.

### DETAILED DESCRIPTION OF THE INVENTION

When any group, ring, substituent or moiety defined herein is substituted, it is typically substituted by Q as defined below.

A C₁₋₆ alkyl group or moiety is linear or branched. A C₁₋₆ alkyl group is typically a C₁₋₄ alkyl group, or a C₄₋₆ alkyl group. Examples of C₁₋₆ alkyl groups and moieties include methyl, ethyl, n-propyl, i-propyl, n-butyl, i-butyl, t-butyl, n-pentyl, i-pentyl (i.e. 3-methylbut-1-yl), t-pentyl (i.e. 2-methylbut-2-yl), neopentyl (i.e. 2,2-dimethylpropan-1-yl), n-hexyl, i-hexyl (i.e. 4-methylpentan-1-yl), t-hexyl (i.e. 3-methylpentan-3-yl) and neopentyl (i.e. 3,3-dimethylbutan-1-yl). For the avoidance of doubt, where two alkyl moieties are present in a group, the alkyl moieties may be the same or different. A C₁₋₆ alkyl group is unsubstituted or substituted, typically by one or more groups Q as defined below. For example, a C₁₋₆ alkyl group is unsubstituted or substituted by 1, 2 or 3 groups Q as defined below.

Q is halo, nitro, —CN, OH, C₁₋₆ alkoxy, C₁₋₆ hydroxyalkyl, C₁₋₆ alkylthio, C₁₋₆ alkyl, C₁₋₆ haloalkyl, C₁₋₄ haloalkoxy, —CO₂R′, —NR′₂, —SR′, —S(═O)R′, —S(═O)₂R′, C₃-C₁₀ cycloalkyl, 5 to 10-membered heterocyclyl, 5- to 12-membered aryl or 5- to 10-membered heteroaryl, wherein each R′ is independently selected from H, C₁₋₆ alkyl, C₃-₁₀ cycloalkyl, 5 to 10-membered heterocyclyl, C₆ - C₁₀ aryl and 5- to 10-membered heteroaryl. For the avoidance of doubt, the alkyl, alkoxy, alkylthio, cycloalkyl, heterocyclyl, aryl and heteroaryl moieties in these definitions are themselves typically unsubstituted.

A C₁₋₆ alkoxy group is linear or branched. It is typically a C₁₋₄ alkoxy group, for example a methoxy, ethoxy, propoxy, i-propoxy, n-propoxy, n-butoxy, sec-butoxy or tert-butoxy group. A C₁₋₆ alkoxy group is unsubstituted or substituted, typically by one or more groups Q as defined above.

A C₁₋₆ alkylthio group is linear or branched. It is typically a C₁₋₄ alkylthio group, for example a methylthio, ethylthio, propylthio, i-propylthio, n-propylthio, n-butylthio, sec-butylthio or tert-butylthio group. A C₁₋₆ alkylthio group is unsubstituted or substituted, typically by one or more groups Q as defined above.

A halogen or halo group is F, Cl, Br or I. Typically it is F or Cl. A C₁₋₆ alkyl group substituted by halogen may be denoted “C₁₋₆ haloalkyl”, which means a C₁₋₆ alkyl group as defined above in which one or more hydrogens is replaced by halo. Likewise a C₁₋₆ alkoxy group substituted by halogen may be denoted “C₁₋₆ haloalkoxy”, which means a C₁₋₆ alkoxy group as defined above in which one or more hydrogens is replaced by halo. Typically, C₁₋₆ haloalkyl or C₁₋₆ haloalkoxy is substituted by 1, 2 or 3 said halogen atoms. Haloalkyl and haloalkoxy groups include perhaloalkyl and perhaloalkoxy groups such as -CX₃ and -OCX₃ wherein X is a halogen, for example —CF₃ —CCl₃ —OCF₃ and —OCCl₃.

A C₁₋₆ hydroxyalkyl group is a C₁₋₆ alkyl group as defined above, substituted by one or more OH groups. Typically, it is substituted by one, two or three OH groups. Preferably, it is substituted by a single OH group.

A C₆ - C₁₀ aryl group is an aromatic carbocyclic group containing from 6 to 10 carbon atoms. It is monocyclic or a fused bicyclic ring system in which an aromatic ring is fused to another aromatic carbocyclic ring. Examples of a C₆ - C₁₀ aryl group include phenyl and naphthyl. When substituted, an aryl group is typically substituted by a group Q as defined above, for instance by 1, 2 or 3, groups selected from a a group Q as defined above. More particularly, a substituted aryl group such as a substituted phenyl group is substituted by 1 or 2 groups selected from C₁-C₆ alkyl, halo, —OR⁸ and —N(R⁸)₂ wherein R⁸ is H or C₁-C₆ alkyl, each R⁸ being the same or different when two are present.

A C₃₋₁₀ cycloalkyl group is a saturated hydrocarbon ring having from 3 to 10 carbon atoms. A C₃₋₁₀ cycloalkyl group may be, for instance, C₃-C₇ cycloalkyl such as cyclopropyl, cyclobutyl, cyclopentyl, cyclohexyl, or cycloheptyl. Typically it is C₃-C₆ cycloalkyl, or C₄-C₆ cycloalkyl, for example cyclobutyl, cyclopentyl or cyclohexyl. In one embodiment it is cyclobutyl. A C₃₋₁₀ cycloalkyl group is unsubstituted or substituted, typically by one or more groups Q as defined above.

A 4- to 10- membered heteroaryl group or moiety is a 4- to 10-membered aromatic heterocyclic group which contains 1, 2, 3, or 4 heteroatoms selected from O, N and S. It is monocyclic or bicyclic. Typically it contains one N atom and 0, 1, 2 or 3 additional heteroatoms selected from O, S and N. It may be, for example, a monocyclic 5- to 7-membered heteroaryl group, for instance a 5- or 6-membered N-containing heteroaryl group. Examples include pyridyl, pyrazinyl, pyrimidinyl, pyridazinyl, furanyl, thienyl, pyrazolidinyl, pyrrolyl, oxadiazolyl, oxazolyl, isoxazolyl, thiazolyl, thiadiazolyl, imidazolyl and pyrazolyl groups. Furanyl, thienyl, imidazolyl, pyridyl and pyrimidyl groups are preferred. It may alternatively be a bicyclic heteroaryl group, for instance an 8- to 10-membered bicyclic heteroaryl group. Examples include quinolyl, isoquinolyl, quinazolyl, quinoxalinyl, indolyl, isoindolyl, indazolyl, imidazopyridazinyl, pyrrolopyridinyl, pyrazolopyrimidinyl and pyrrolopyrimidinyl. When substituted, a heteroaryl group (monocyclic or bicyclic) is typically substituted by one or more, e.g. 1, 2 or 3, groups selected from C₁₋₄ alkyl and a group Q as defined above.

A 4- to 10-membered heterocyclyl group is a monocyclic or bicyclic non-aromatic, saturated or unsaturated ring system containing 5 to 10 carbon atoms and at least one atom or group selected from N, O, S, SO, SO₂ and CO, more typically N or O. When the ring system is bicyclic, one ring may be saturated and one ring unsaturated. Typically, it is a C₄₋₁₀ ring system in which 1, 2 or 3 of the carbon atoms in the ring are replaced with an atom or group selected from O, S, SO₂, CO and NH. More typically it is a monocyclic ring, preferably a monocyclic C₄-C₆ ring. Examples include piperidyl, piperidin-2,6-dionyl, piperidin-2-onyl, piperazinyl, morpholinyl, thiomorpholinyl, S,S-dioxothiomorpholinyl, 1,3-dioxolanyl, pyrrolidinyl, imidazol-2-onyl, pyrrolidin-2-onyl, oxetanyl, tetrahydrofuranyl and tetrahydropyranyl moieties.

For the avoidance of doubt, although the above definitions of heteroaryl and heterocyclyl groups refer to an “N” atom which can be present in the ring, it will be evident to a skilled chemist that any such N atom will be protonated (or will carry a substituent as defined above) if it is attached to each of its adjacent ring atoms via a single bond. Such protonated forms are embraced within the present definitions of heteroaryl and heterocyclyl groups.

In formula (Ib) or (I), R¹ is typically H or F. Y is typically O, S or SO₂. More typically Y is O or SO₂. Most typically Y is O.

When in formula (Ib) or (I) group R² is —NR¹¹R¹² in which R¹¹ and R¹² form, together with the N atom to which they are attached, a morpholine ring which is optionally bridged by a

—CH₂— group linking two ring carbon atoms that are positioned para to each other, the group has the following structure (c) or (d):

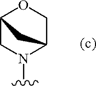

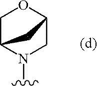

In one embodiment of formula (Ib) as defined above, herein denoted formula (Ic):
R¹ is H or F;
Y is O, S or SO₂;
each of V and X is CH and W is N or CH;
R² is selected from C₁-C₆ alkyl, halo, —NR₂ and —NHR″, in which R and R″ are as defined above for formula (I);
n is 0, 1 or 2; and
each of R³ to R¹⁰ is independently selected from H, C₁-C₆ alkyl, halo and —NR¹³R¹⁴ wherein R¹³ and R¹⁴ form, together with the N atom to which they are attached, a morpholine ring; or
any two of R³ to R¹⁰ that bond to the same carbon atom form a C₃-C₆ spiro ring and the rest of R³ to R¹⁰ are H; or any two of R³ to R¹⁰ that bond to non-adjacent carbon atoms form a C₁-C₃ bridgehead group linking the carbon atoms to which they are bonded and the rest of R³ to R¹⁰ are H.

In formula (Ic), Y is typically O.

In a particular embodiment of formula (I) as defined above, herein denoted formula (Ia):
R¹ is H or F;
Y is O or SO₂;
each of V and X is CH and W is N or CH;
R² is selected from C₁-C₆ alkyl, halo, —NR₂ and —NHR″, in which R and R″ are as defined above for formula (I);
n is 0, 1 or 2; and
each of R³ to R¹⁰ is independently selected from H, C₁-C₆ alkyl and halo; or any two of R³ to R¹⁰ that bond to the same carbon atom form a C₃-C₆ spiro ring and the rest of R³ to R¹⁰ are H; or any two of R³ to R¹⁰ that bond to non-adjacent carbon atoms form a C₁-C₃ bridgehead group linking the carbon atoms to which they are bonded and the rest of R³ to R¹⁰ are H.

In formula (Ia), Y is typically O.

In another embodiment, compounds of the invention have the following formula (I′):

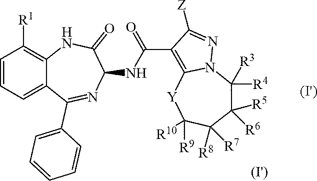

wherein each of R¹, Y and R³ to R¹⁰ is as defined above for formula (Ib), (Ic), (I) or (Ia) and Z is selected from the following structures:

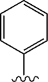

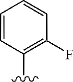

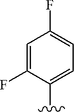

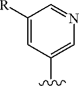

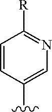

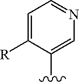

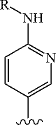

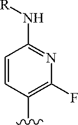

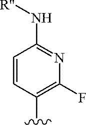

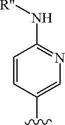

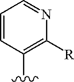

in which R and R″ are as defined above for formula (Ib) or (I). Typically R is H or C₁-C₃ alkyl and R″ is cyclopropyl.

In formula (I′), R¹ is typically H or F. Y is typically O, S or SO₂. More typically Y is O or SO₂. Most typically Y is O.

In formulae (Ib), (Ic) and (I′), R³ to R¹⁰ may take the following values:
each of R³ to R¹⁰ is H; or
one or two of R³ to R¹⁰ are C₁-C₃ alkyl, halo, typically F, or —NR¹³R¹⁴ wherein R¹³ and R¹⁴ form, together with the N atom to which they are attached, a morpholine ring, and the rest of R³ to R¹⁰ are H; or
R³ and R¹⁰ form a C₁ or C₂ bridgehead group linking the carbon atoms to which they are attached, and each of R⁴ to R⁹ is H.

In a particular embodiment, in formulae (I), (Ia) and (I′), R³ to R¹⁰ may take the following values:
each of R³ to R¹⁰ is H; or
one or two of R³ to R¹⁰ are C₁-C₃ alkyl or halo, typically F, and the rest of R³ to R¹⁰ are H; or
R³ and R¹⁰ form a C₁ or C₂ bridgehead group linking the carbon atoms to which they are attached, and each of R⁴ to R⁹ is H.

More typically in formulae (Ib), (Ic) and (I′), R³ to R¹⁰ may take the following values:
each of R³ to R¹⁰ is H; or
-one of R³ and R¹⁰ is C₁-C₃ alkyl and the rest of R³ to R¹⁰ are H; or
each of R³ to R⁸ is H and each of R⁹ and R¹⁰ is C₁-C₃ alkyl; or
each of R⁴ to R⁹ is H and each of R³ and R¹⁰ is C₁-C₃ alkyl; or
each of R³ to R⁶, R⁹ and R¹⁰ is H and each of R⁷ and R⁸ is halo; or
one of R⁷ and R⁸ is —NR¹³R¹⁴ wherein R¹³ and R¹⁴ form, together with the N atom to which they are attached, a morpholine ring, and the rest of R³ to R¹⁰ are H; or
R³ and R¹⁰ form a C₁ or C₂ bridgehead group linking the carbon atoms to which they are attached, and each of R⁴ to R⁹ is H.

In a particular embodiment, more typically in formulae (I), (Ia) and (I′), R³ to R¹⁰ may take the following values:
each of R³ to R¹⁰ is H; or
one of R³ and R¹⁰ is C₁-C₃ alkyl and the rest of R³ to R¹⁰ are H; or
each of R³ to R⁸ is H and each of R⁹ and R¹⁰ is C₁-C₃ alkyl; or
each of R⁴ to R⁹ is H and each of R³ and R¹⁰ is C₁-C₃ alkyl; or
each of R³ to R⁶, R⁹ and R¹⁰ is H and each of R⁷ and R⁸ is halo, typically F; or
R³ and R¹⁰ form a C₁ or C₂ bridgehead group, typically a C₁ bridgehead group, linking the carbon atoms to which they are attached, and each of R⁴ to R⁹ is H.

The bond linking each of R³ to R¹⁰ to the adjacent C atom may be oriented above or below the plane of the seven-membered ring, i.e. depicted as

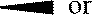

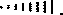

For instance, in any of formulae (Ib), (Ic), (I), (Ia) and (I′) as defined above, with any of the values of R³ to R¹⁰ set out above, R³ may be

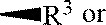

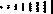

R³. Similarly and independently, R¹⁰ may be

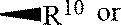

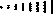

In formulae (Ib), (Ic), (I), (Ia) and (I′) having any of the foregoing values of R³ to R¹⁰, and any orientations of R³ to R¹⁰, Y is typically O, S or SO₂. More typically Y is O or SO₂. Most typically Y is O.

Specific compounds of the invention include the following:
N-[(3S)-2-oxo-5-phenyl-1,3-dihydro-1,4-benzodiazepin-3-yl]-2-phenyl-5,6,7,8-tetrahydropyrazolo[5,1-b][1,3]oxazepine-3-carboxamide;
N-[(3S)-9-Fluoro-2-oxo-5-phenyl-1,3-dihydro-1,4-benzodiazepin-3-yl]-2-(2-fluorophenyl)-5,6,7,8-tetrahydropyrazolo[5,1-b][1,3]oxazepine-3-carboxamide;
2-(5-Methylpyridin-3-yl)-N-[(3 S)-2-oxo-5-phenyl-1,3-dihydro-1,4-benzodiazepin-3-yl]-5,6,7,8-tetrahydropyrazolo[5,1-b][1,3]oxazepine-3-carboxamide;
N-[(3S)-9-Fluoro-2-oxo-5-phenyl-1,3-dihydro-1,4-benzodiazepin-3-yl]-2-(5-methylpyridin-3-yl)-5,6,7,8-tetrahydropyrazolo[5,1-b][1,3]oxazepine-3-carboxamide;
2-(2-Fluorophenyl)-5,5-dimethyl-N-[(3S)-2-oxo-5-phenyl-1,3-dihydro-1,4-benzodiazepin-3-yl]-7,8-dihydro-6H-pyrazolo[5,1-b][1,3]oxazepine-3-carboxamide;
N-[(3S)-9-Fluoro-2-oxo-5-phenyl-1,3-dihydro-1,4-benzodiazepin-3-yl]-2-(2-fluorophenyl)-5,5-dimethyl-7,8-dihydro-6H-pyrazolo[5, 1-b][1,3]oxazepine-3-carboxamide;
2-(2-Fluorophenyl)-N-[(3 S)-2-oxo-5-phenyl-1,3-dihydro-1,4-benzodiazepin-3-yl]-5,6,7,8-tetrahydropyrazolo[5,1-b][1,3]oxazepine-3-carboxamide;
N-[(3S)-9-Fluoro-2-oxo-5-phenyl-1,3-dihydro-1,4-benzodiazepin-3-yl]-2-phenyl-5,6,7,8-tetrahydropyrazolo[5,1-b][1,3]oxazepine-3-carboxamide;
2-[6-(Cyclopropylamino)-2-fluoropyridin-3-yl]-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
2-[6-(Ethylamino)-2-fluoropyridin-3-yl]-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
2-[6-(Ethylamino)-2-fluoropyridin-3-yl]-N-[(3S)-9-fluoro-2-oxo-5-phenyl-1,3-dihydro-1,4-benzodiazepin-3-yl]-5-methyl-5,6,7,8-tetrahydropyrazolo[5,1-b][1,3]oxazepine-3-carboxamide;
2-[2-Fluoro-6-(propan-2-ylamino)pyridin-3-yl]-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
N-[(3S)-9-fluoro-2-oxo-5-phenyl-1,3-dihydro-1,4-benzodiazepin-3-yl]-2-[2-fluoro-6-(propan-2-ylamino)pyridin-3-yl]-5-methyl-5,6,7,8-tetrahydropyrazolo[5, 1-b][1,3]oxazepine-3-carboxamide;
2-(2-Fluorophenyl)-8-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
N-[(3S)-9-fluoro-2-oxo-5-phenyl-1,3-dihydro-1,4-benzodiazepin-3-yl]-2-(2-fluorophenyl)-8-methyl-5,6,7,8-tetrahydropyrazolo[5,1-b][1,3]oxazepine-3-carboxamide;
2-(2,4-Difluorophenyl)-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
2-(2,4-Difluorophenyl)-N-[(3S)-9-fluoro-2-oxo-5-phenyl-1,3-dihydro-1,4-benzodiazepin-3-yl]-5-methyl-5,6,7,8-tetrahydropyrazolo[5,1-b][1,3]oxazepine-3-carboxamide;
2-(2-Fluorophenyl)-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
N-((S)-9-fluoro-2-oxo-5-phenyl-2,3-dihydro-1H-benzo[b]azepin-3-yl)-2-(2-fluorophenyl)-5,6,7,8-tetrahydro-5,8-methanopyrazolo[5,1-b][1,3]oxazepine-3-carboxamide;
N-[(3S)-9-fluoro-2-oxo-5-phenyl-2,3-dihydro-1H-1,4-benzodiazepin-3-yl]-2-(2-fluorophenyl)-5H,6H,7H,8H-pyrazolo[3,2-b][1,3]thiazepine-3-carboxamide;
N-[(3S)-9-fluoro-2-oxo-5-phenyl-2,3-dihydro-1H-1,4-benzodiazepin-3-yl]-2-(2-fluorophenyl)-4,4-dioxo-5H,6H,7H,8H-4λ⁶-pyrazolo[3,2-b][1,3]thiazepine-3-carboxamide;
2-(6-Ethylpyridin-3-yl)-6,6-difluoro-N-[(3S)-9-fluoro-2-oxo-5-phenyl-2,3-dihydro-1H-1,4-benzodiazepin-3-yl]-5H,6H,7H,8H-pyrazolo[3,2-b][1,3]oxazepine-3-carboxamide;
6,6-Difluoro-N-[(3 S)-9-fluoro-2-oxo-5-phenyl-2,3-dihydro-1H-1,4-benzodiazepin-3-yl]-2-{6-[(propan-2-yl)amino]pyridin-3-yl}-5H,6H,7H,8H-pyrazolo[3,2-b][1,3]oxazepine-3-carboxamide;
5-Ethyl-2-(2-fluorophenyl)-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
5-Ethyl-N-[(3 S)-9-fluoro-2-oxo-5-phenyl-1,3-dihydro-1,4-benzodiazepin-3-yl]-2-(2-fluorophenyl)-5,6,7,8-tetrahydropyrazolo[5,1-b][1,3]oxazepine-3-carboxamide;
5-Ethyl-2-(6-methylpyridin-3-yl)-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
5-Ethyl-N-[(3 S)-9-fluoro-2-oxo-5-phenyl-1,3-dihydro-1,4-benzodiazepin-3-yl]-2-(6-methylpyridin-3-yl)-5,6,7,8-tetrahydropyrazolo[5,1-b][1,3]oxazepine-3-carboxamide;
4-(6-Ethylpyridin-3-yl)-N-[(3S)-9-fluoro-2-oxo-5-phenyl-2,3-dihydro-1H-1,4-benzodiazepin-3-yl]-7-oxa-2,3-diazatricyclo[6.2.1.0^{2,6}]undeca-3,5-diene-5-carboxamide;
N-[(3S)-9-fluoro-2-oxo-5-phenyl-2,3-dihydro-1H-1,4-benzodiazepin-3-yl]-4-{6-(propan-2-yl)amino]pyridin-3-yl}-7-oxa-2,3-diazatricyclo[6.2.1.0^{2,6}]undeca-3,5-diene-5-carboxamide;
6,6-Difluoro-N-[(3S)-9-fluoro-2-oxo-5-phenyl-2,3-dihydro-1H-1,4-benzodiazepin-3-yl]-2-(2-fluorophenyl)-5H,6H,7H,8H-pyrazolo[3,2-b][1,3]oxazepine-3-carboxamide;
2-(2-Fluorophenyl)-6-morpholin-4-yl-N-[(3R)-9-fluoro-2-oxo-5-phenyl-1,3-dihydro-1,4-benzodiazepin-3-yl]-5,6,7,8-tetrahydropyrazolo[5,1-b][1,3]oxazepine-3-carboxamide;
4-(2-Fluoro-4-methylsulfonylphenyl)-N-[(3S)-9-fluoro-2-oxo-5-phenyl-1,3-dihydro-1,4-benzodiazepin-3-yl]-7-oxa-2,3-diazatricyclo[6.2.1.02,6]undeca-3,5-diene-5-carboxamide;
(5R*)-2-[6-(ethylamino)-2-fluoropyridin-3-yl]-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
(5S*)-2-[6-(ethylamino)-2-fluoropyridin-3-yl]-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
(5R)-2-[6-(ethylamino)-2-fluoropyridin-3-yl]-N-[(3S)-9-fluoro-2-oxo-5-phenyl-1,3-dihydro-1,4-benzodiazepin-3-yl]-5-methyl-5,6,7,8-tetrahydropyrazolo[5,1-b][1,3]oxazepine-3-carboxamide;
(5S*)-2-[6-(ethylamino)-2-fluoropyridin-3-yl]-N-[(3S)-9-fluoro-2-oxo-5-phenyl-1,3-dihydro-1,4-benzodiazepin-3-yl]-5-methyl-5,6,7,8-tetrahydropyrazolo[5,1-b][1,3]oxazepine-3-carboxamide;
(5R*)-2-[2-fluoro-6-(propan-2-ylamino)pyridin-3-yl]-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
(5S*)-2-[2-fluoro-6-(propan-2-ylamino)pyridin-3-yl]-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
(5R*)-N-[(3S)-9-fluoro-2-oxo-5-phenyl-1,3-dihydro-1,4-benzodiazepin-3-yl]-2-[2-fluoro-6-(propan-2-ylamino)pyridin-3-yl]-5-methyl-5,6,7,8-tetrahydropyrazolo[5,1-b][1,3]oxazepine-3-carboxamide;
(5S*)-N-[(3S)-9-fluoro-2-oxo-5-phenyl-1,3-dihydro-1,4-benzodiazepin-3-yl]-2-[2-fluoro-6-(propan-2-ylamino)pyridin-3-yl]-5-methyl-5,6,7,8-tetrahydropyrazolo[5,1-b][1,3]oxazepine-3-carboxamide;
(8R*)-2-(2-Fluorophenyl)-8-methyl-N-(2-oxo-5-phenyl-1,3-dihydro-1,4-benzodiazepin-3-yl)-5,6,7,8-tetrahydropyrazolo[5,1-b][1,3]oxazepine-3-carboxamide;
(8S*)-2-(2-Fluorophenyl)-8-methyl-N-(2-oxo-5-phenyl-1,3-dihydro-1,4-benzodiazepin-3-yl)-5,6,7,8-tetrahydropyrazolo[5,1-b][1,3]oxazepine-3-carboxamide;
(8R*)-N-(9-fluoro-2-oxo-5-phenyl-1,3-dihydro-1,4-benzodiazepin-3-yl)-2-(2-fluorophenyl)-8-methyl-5,6,7,8-tetrahydropyrazolo[5,1-b][1,3]oxazepine-3-carboxamide;
(8S*)-N-(9-fluoro-2-oxo-5-phenyl-1,3-dihydro-1,4-benzodiazepin-3-yl)-2-(2-fluorophenyl)-8-methyl-5,6,7,8-tetrahydropyrazolo[5,1-b][1,3]oxazepine-3-carboxamide;
(5R*)-2-(2,4-difluorophenyl)-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
(5S*)-2-(2,4-difluorophenyl)-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
(5R*)-2-(2,4-difluorophenyl)-N-[(3 S)-9-fluoro-2-oxo-5-phenyl-1,3-dihydro-1,4-benzodiazepin-3-yl]-5-methyl-5,6,7,8-tetrahydropyrazolo[5,1-b][1,3]oxazepine-3-carboxamide;
(5S*)-2-(2,4-difluorophenyl)-N-[(3S)-9-fluoro-2-oxo-5-phenyl-1,3-dihydro-1,4-benzodiazepin-3-yl]-5-methyl-5,6,7,8-tetrahydropyrazolo[5,1-b][1,3]oxazepine-3-carboxamide;
(1S*,8R*)-N-[(3 S)-9-fluoro-2-oxo-5-phenyl-2,3-dihydro-1H-1,4-benzodiazepin-3-yl]-4-(2-fluorophenyl)-7-oxa-2,3-diazatricyclo[6.2.1.0^{2,6}]undeca-3,5-diene-5-carboxamide;
(1R*,8S*)-N-[(3 S)-9-fluoro-2-oxo-5-phenyl-2,3-dihydro-1H-1,4-benzodiazepin-3-yl]-4-(2-fluorophenyl)-7-oxa-2,3-diazatricyclo[6.2.1.0^{2,6}]undeca-3,5-diene-5-carboxamide;
(5R*)-5-ethyl-2-(2-fluorophenyl)-N-[(3S)-2-oxo-5-phenyl-2,3-dihydro-1H-1,4-benzodiazepin-3-yl]-5H,6H,7H,8H-pyrazolo[3,2-b][1,3]oxazepine-3-carboxamide;
(5S*)-5-ethyl-2-(2-fluorophenyl)-N-[(3S)-2-oxo-5-phenyl-2,3-dihydro-1H-1,4-benzodiazepin-3-yl]-5H,6H,7H,8H-pyrazolo[3,2-b][1,3]oxazepine-3-carboxamide;
(5R*)-5-ethyl-2-(2-fluorophenyl)-N-[(3S)-9-fluoro-2-oxo-5-phenyl-1,3-dihydro-1,4-benzodiazepin-3-yl]-5,6,7,8-tetrahydropyrazolo[5,1-b][1,3]oxazepine-3-carboxamide;
(5S*)-5-ethyl-2-(2-fluorophenyl)-N-[(3S)-9-fluoro-2-oxo-5-phenyl-1,3-dihydro-1,4-benzodiazepin-3-yl]-5,6,7,8-tetrahydropyrazolo[5,1-b][1,3]oxazepine-3-carboxamide;
(5R*)-5-ethyl-2-(6-methylpyridin-3-yl)-N-[(3 S)-2-oxo-5-phenyl-2,3-dihydro-1H-1,4-benzodiazepin-3-yl]-5H,6H,7H,8H-pyrazolo[3,2-b][1,3]oxazepine-3-carboxamide;
(5S*)-5-ethyl-2-(6-methylpyridin-3-yl)-N-[(3S)-2-oxo-5-phenyl-2,3-dihydro-1H-1,4-benzodiazepin-3-yl]-5H,6H,7H,8H-pyrazolo[3,2-b][1,3]oxazepine-3-carboxamide;
(5R*)-5-ethyl-N-[(3S)-9-fluoro-2-oxo-5-phenyl-2,3-dihydro-1H-1,4-benzodiazepin-3-yl]-2-(6-methylpyridin-3-yl)-5H,6H,7H,8H-pyrazolo[3,2-b][1,3]oxazepine-3-carboxamide;
(5S*)-5-ethyl-N-[(3S)-9-fluoro-2-oxo-5-phenyl-2,3-dihydro-1H-1,4-benzodiazepin-3-yl]-2-(6-methylpyridin-3-yl)-5H,6H,7H,8H-pyrazolo[3,2-b][1,3]oxazepine-3-carboxamide;
(1S*,8R*)-4-(6-ethylpyridin-3-yl)-N-[(3S)-9-fluoro-2-oxo-5-phenyl-2,3-dihydro-1H-1,4-benzodiazepin-3-yl]-7-oxa-2,3-diazatricyclo[6.2.1.02,6]undeca-3,5-diene-5-carboxamide;
(1R*,8S*)-4-(6-ethylpyridin-3-yl)-N-[(3S)-9-fluoro-2-oxo-5-phenyl-2,3-dihydro-1H-1,4-benzodiazepin-3-yl]-7-oxa-2,3-diazatricyclo[6.2.1.02,6]undeca-3,5-diene-5-carboxamide;
(1S*,8R*)-N-[(3S)-9-fluoro-2-oxo-5-phenyl-2,3-dihydro-1H-1,4-benzodiazepin-3-yl]-4-{6-[(propan-2-yl)amino]pyridin-3-yl}-7-oxa-2,3-diazatricyclo[6.2.1.02,6]undeca-3,5-diene-5-carboxamide;
(1R*,8S*)-N-[(3S)-9-fluoro-2-oxo-5-phenyl-2,3-dihydro-1H-1,4-benzodiazepin-3-yl]-4-{6-[(propan-2-yl)amino]pyridin-3-yl}-7-oxa-2,3-diazatricyclo[6.2.1.02,6]undeca-3,5-diene-5-carboxamide;
and the pharmaceutically acceptable salts thereof.

The compounds of the invention may contain asymmetric or chiral centres, and therefore exist in different stereoisomeric forms. It is intended that all stereoisomeric forms of the compounds of the invention, including but not limited to, diastereomers, enantiomers and atropisomers, as well as mixtures thereof such as racemic mixtures, form part of the present invention. Compounds of formula (Ib) or (I) containing one or more chiral centre may be used in enantiomerically or diastereoisomerically pure form, or in the form of a mixture of isomers.

The present invention embraces all geometric and positional isomers of compounds of the invention as defined above. For example, if a compound of the invention incorporates a double bond or a fused ring, the cis- and trans-forms, as well as mixtures thereof, are embraced within the scope of the invention. Both the single positional isomers and mixture of positional isomers are also within the scope of the present invention.

The compounds of the present invention may exist in unsolvated as well as solvated forms with pharmaceutically acceptable solvents such as water, ethanol, and the like, and it is intended that the invention embrace both solvated and unsolvated forms.

The compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol tautomerizations. Valence tautomers include interconversions by reorganization of some of the bonding electrons.

The present invention embraces all isotopologues of compounds of the invention as defined above. Thus, any atom present in a compound of the invention as defined above, or in any intermediate or starting compound, may be present in any available naturally-occurring isotopic form. For instance, a carbon atom may be ¹²C or ¹³C. A hydrogen atom may be ¹H or ²H (deuterium). A compound of the invention as defined above may thus be prepared in deuterated form, with one or more hydrogen atoms present as ²H. Any hydrogen atoms or combination thereof may be present as deuterium.

Compounds of the invention can be prepared by the synthetic methods described in the Examples that follow, or by analogy with such methods using appropriate starting materials and methodologies familiar to the skilled chemist. The preparation typically comprises, as a final step, an amide coupling reaction in which the central amide linkage in formula (Ib) or (I) as defined above is formed. Compounds of the invention may thus be prepared by the amide coupling reactions designated Procedure A, Procedure B and Procedure C in the Examples, or by analogy with any of Procedure A, Procedure B and Procedure C using appropriate synthetic intermediates. Such intermediates may be prepared by analogous methods to those described in the Preparatory Examples.

A benzodiazepine derivative of formula (Ib) or (I) can be converted into a pharmaceutically acceptable salt thereof, and a salt can be converted into the free compound, by conventional methods. For instance, a benzodiazepine derivative of formula (Ib) or (I) can be contacted with a pharmaceutically acceptable acid to form a pharmaceutically acceptable salt. A pharmaceutically acceptable salt is a salt with a pharmaceutically acceptable acid or base.

Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic or p-toluenesulphonic acid.

Pharmaceutically acceptable bases include alkali metal (e.g. sodium or potassium) and alkali earth metal (e.g. calcium or magnesium) hydroxides and organic bases such as alkyl amines, aralkyl amines and heterocyclic amines.

Compounds of the present invention have been found in biological tests to be inhibitors of respiratory syncytial virus (RSV). They possess a combination of potent anti-RSV activity with good bioavailability and good physicochemical properties. This combination of properties makes the compounds therapeutically useful and superior as drug candidates to many compounds disclosed in the prior art references discussed earlier.

Accordingly, the present invention further provides a compound which is a benzodiazepine derivative of formula (Ib) or (I), as defined above, or a pharmaceutically acceptable salt thereof, for use in a method of treating the human or animal body by therapy.

The invention also provides a compound of the invention as defined above for use in a method treating or preventing an RSV infection. Still further, the present invention provides the use of a compound of the invention as defined above in the manufacture of a medicament for use in treating or preventing an RSV infection. A subject suffering from or susceptible to an RSV infection may thus be treated by a method comprising the administration thereto of a compound of the invention as defined above. The condition of the subject may thereby be improved or ameliorated.

The RSV infection is typically a respiratory tract infection. The RSV infection may be an infection in a child, for instance a child under ten years of age or an infant under two years of age. In one embodiment the invention provides a compound as defined above for use in treating or preventing an RSV infection in paediatric patients. Alternatively the infection may be an infection in a mature or elderly adult, for instance an adult over 60 years of age, an adult over 70 years of age, or an adult over 80 years of age. The invention further provides a compound for use in treating or preventing an RSV infection in geriatric patients.

The RSV infection may be an infection in an immunocompromised individual or an individual suffering from COPD or CHF. In another embodiment, the RSV infection is an infection in a non-compromised individual, for instance an individual who is otherwise healthy.

A compound of the present invention can be administered in a variety of dosage forms, for example orally such as in the form of tablets, capsules, sugar- or film-coated tablets, liquid solutions or suspensions or parenterally, for example intramuscularly, intravenously or subcutaneously. The compound may therefore be given by injection, infusion, or by inhalation or nebulaisation. The compound is preferably given by oral administration.

The dosage depends on a variety of factors including the age, weight and condition of the patient and the route of administration. Daily dosages can vary within wide limits and will be adjusted to the individual requirements in each particular. Typically, however, the dosage adopted for each route of administration when a compound is administered alone to adult humans is 0.0001 to 650 mg/kg, most commonly in the range of 0.001 to 10 mg/kg, body weight, for instance 0.01 to 1 mg/kg. Such a dosage may be given, for example, from 1 to 5 times daily. For intravenous injection a suitable daily dose is from 0.0001 to 1 mg/kg body weight, preferably from 0.0001 to 0.1 mg/kg body weight. A daily dosage can be administered as a single dosage or according to a divided dose schedule.

A unit dose form such as a tablet or a capsule will usually contain 1-250 mg of active ingredient. For example, a compound of formula (Ib) or (I) could be administered to a human patient at a dose of between 100-250 mg either once a day, twice or three times a day. For example, a compound of formula (Ib) or (I) could be administered to a human patient at a dose of between 100-250 mg either once a day, twice or three times a day.

The compounds of formula (Ib) or (I) and pharmaceutically acceptable salts thereof may be used on their own. Alternatively, they may be administered in the form of a pharmaceutical composition. The present invention therefore also provides a pharmaceutical composition comprising a compound of formula (Ib) or (I) or a pharmaceutically acceptable salt thereof as hereinbefore defined, in association with a pharmaceutically acceptable adjuvant, diluent or carrier. Conventional procedures for the selection and preparation of suitable pharmaceutical formulations are described in, for example, “Pharmaceuticals - The Science of Dosage Form Designs”, M. E. Aulton, Churchill Livingstone, 1988.

Depending on the mode of administration, the pharmaceutical composition will preferably comprise from 0.05 to 99%w (percent by weight), more preferably from 0.05 to 80%w, still more preferably from 0.10 to 70%w, and even more preferably from 0.10 to 50%w, of active ingredient, all percentages by weight being based on total composition.

The invention further provides a process for the preparation of a pharmaceutical composition of the invention which comprises mixing a compound of formula (Ib) or (I) or a pharmaceutically acceptable salt thereof as hereinbefore defined with a pharmaceutically acceptable adjuvant, diluent or carrier.

The compounds of the invention may be administered in a variety of dosage forms. Thus, they can be administered orally, for example as tablets, troches, lozenges, aqueous or oily suspensions, solutions, dispersible powders or granules. The compounds of the invention may also be administered parenterally, whether subcutaneously, intravenously, intramuscularly, intrasternally, transdermally, by infusion techniques or by inhalation or nebulisation. The compounds may also be administered as suppositories.

Solid oral forms of the pharmaceutical composition of the invention may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulfates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride. Further suitable carriers for suspensions include sterile water, hydroxypropylmethyl cellulose (HPMC), polysorbate 80, polyvinylpyrrolidone (PVP), aerosol AOT (i.e. sodium 1,2-bis(2-ethylhexoxycarbonyl)ethanesulphonate), pluronic F127 and/or captisol (i.e. sulfobutylether-beta-cyclodextrin).

The compounds of the invention may, for example, be formulated as aqueous suspensions in a carrier selected from:
(i) 0.5% w/v hydroxypropylmethyl cellulose (HPMC)/0.1% w/v polysorbate 80;
(ii) 0.67% w/v polyvinylpyrrolidone (PVP)/0.33% w/v aerosol AOT (sodium 1,2-bis(2-ethylhexoxycarbonyl)ethanesulphonate);
(iii) 1% w/v pluronic F 127; and
(iv) 0.5% w/v polysorbate 80.

The carriers may be prepared by standard procedures known to those of skill in the art. For example, each of the carriers (i) to (iv) may be prepared by weighing the required amount of excipient into a suitable vessel, adding approximately 80% of the final volume of water and magnetically stirring until a solution is formed. The carrier is then made up to volume with water. The aqueous suspensions of compounds of formula (Ib) or (I) may be prepared by weighing the required amount of a compound of formula (Ib) or (I) into a suitable vessel, adding 100% of the required volume of carrier and magnetically stirring.

Solutions for injection or infusion may contain as carrier, for example, sterile water or preferably they may be in the form of sterile, aqueous, isotonic saline solutions.

The compounds of the invention may also be administered in conjunction with other compounds used for the treatment of viral infections. Thus, the invention further relates to combination therapies wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment or prevention of a viral infection, particularly infection by RSV.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

Suitable therapeutic agents for use in the combination therapies include
(i) RSV fusion inhibitors
(ii) other RSV nucleocapsid (N)-protein inhibitors;
(iii) other RSV protein inhibitors, such as those that inhibit the phosphoprotein (P) protein and large (L) protein;
(iv) nucleoside or polymerase inhibitors that inhibit the L protein;
(v) anti-RSV monoclonal antibodies, such as the F-protein antibodies;
(vi) immunomodulating toll-like receptor compounds;
(vii) other respiratory virus anti-virals, such as anti-influenza and anti-rhinovirus compounds; and/or
(viii) anti-inflammatory compounds.

The RSV nucleocapsid (N)-protein plays a pivotal role in viral transcription and replication, mediating the interaction between the genomic RNA and the virally encoded RNA-dependent RNA polymerase. The RSV P- and L-proteins are components of RSV’s virally encoded RNA-dependent RNA polymerase.

According to a further aspect of the invention, there is provided a compound of the formula (Ib) or (I) or a pharmaceutically acceptable salt thereof as hereinbefore defined in combination with one or more of the therapeutic agents listed as (i) to (vi) above for use in the treatment of RSV.

The Examples that follow serve to illustrate the invention further. The Preparatory Examples relate to the preparation of starting materials and intermediates used to prepare the compounds of the Examples. Neither the Examples nor the Preparatory Examples limit the invention in any way.

### EXAMPLES

Reagents were obtained from commercial sources and were used without further purification. All temperatures are in °C. TLC was performed on aluminium backed silica gel plates with fluorescence indicator at 254 nM (median pore size 60 Å). Flash column chromatography was performed using a Biotage Isolera One system using KP-Sil or Ultra silica gel columns or an Isco CombiFlash Rf using FlashPure or RediSep Rf/RediSep Rf Gold silica gel columns.

Analytical chiral HPLC (HPLC Method 1A) was performed at ambient column temperature on an Agilent 1100 HPLC (UV detection at 230 nM) with a ChiralPAK IC column (2.1 × 150 mm; particle size 3 µm) with a flow rate of 0.4 mL/min and a 10 min run time. Preparative HPLC was performed at ambient column temperature by the following methods. HPLC Method 1 was performed on a Waters purification system (UV detection at 210-400 nm) with a Waters Sunfire (19 × 100 mm; 5 µm) column at 28 mL/min. HPLC Method 2 was performed on a Gilson HPLC system (UV detection at 230 nm) with a ChiralPAK IC (20 × 250 mm; 5 µm) at 15 mL/min. HPLC Method 3 was performed on an Agilent 1260 Infinity II Prep HPLC (UV detection at 210-400 nM) with a XBridge BEH C18 (30 × 100 mm; 5 µm) at 42 mL/min. HPLC Method 4 was performed on an Agilent 1260 Infinity IIPrep HPLC system (UV detection at 210-400 nm) with a Waters XSelect CSH (30 × 100 mm; 5 µm) column at 42 mL/min.

Preparative Chiral SFC was performed using a Waters SFC prep 15 (UV detection by DAD at 210 - 400 nm; flow rate 15 mL/min; column temperature 40° C.; 120 bar back pressure) and the following columns: SFC Method 1: Chiralpak® IA (Daicel Ltd.) (1 × 25 cm; 5 µm); SFC Method 2: Chiralpak® IC (Daicel Ltd.) (1 × 25 cm; 5 µm); SFC Method 3: Phenomenex Lux® Cellulose-4 (1 × 25 cm; 5 µm).

Analytical Chiral SFC was performed using a Waters SFC ACQUITY UPC² (UV detection by DAD at 220 - 400 nm; flow rate 1.5 mL/min; column temperature 40° C.; 1750 psi back pressure) with 3 min run time on the following columns, unless otherwise noted. SFC Method 1A: ChiralPAK IA-3 (Daicel Ltd.) (2.1 × 150 mm; 3 µm); SFC Method 2A: ChiralPAK IC-3 (Daicel Ltd.) (2.1 × 150 mm, 3 µm); SFC Method 3A: Lux® Cellulose-4, LC Column (150 × 4.6 mm, 3 µm). SFC Method 4A: Chiralpak IA, (250 × 4.6 mm, 5 µm), flow rate 4 mL/min. SFC Method 5A: Chiralpak IC, (250 × 4.6 mm, 5 µm), flow rate 4 mL/min.

NMR spectra were recorded on a 400 or 500 MHz spectrometer at ambient probe temperature (nominal 298 K). Chemical shifts (δ) are given in ppm and calibrated by using the residual peak of the solvent as the internal standard (CDCl₃, δ = 7.26 ppm; DMSO-d₆, δ = 2.50 ppm). Coupling constants are given in Hertz (Hz). LRMS were recorded using an Advion Plate Express expression^{L} compact mass spectrometer equipped with an APCI ion source.

LCMS analysis was performed using a Waters Acquity UPLC with either a CSH C18 or BEH C18 column (2.1 × 30 mm) at 40° C. at 0.77 mL/min with a linear 5-95% acetonitrile gradient appropriate for the lipophilicity of the compound over 1, 3 or 10 minutes. The aqueous portion of the mobile phase was 0.1% formic acid (CSH C18 column) or 10 mM ammonium bicarbonate (BEH C18 column). LC-UV chromatograms were recorded using a Waters Acquity photodiode array detector between 210 and 400 nm. Mass spectra were recorded using a Waters Acquity QDa detector with ESI switching between positive and negative ion mode.
Method A: 3 min Acidic
Method B: 1 min Acidic
Method C: 10 min Acidic

Preparatory examples (3S)-3-amino-5-phenyl-1,3-dihydro-1,4-benzodiazepin-2-one and (3S)-3-amino-9-fluoro-5-phenyl-1,3-dihydro-1,4-benzodiazepin-2-one were prepared using methods described in WO/2004/026843, WO/2005/090319, and WO/2017/015449.

Abbreviations

| | |
|---|---|
| APCI | Atmospheric pressure chemical ionization |
| DIPEA | N,N-Diisopropylethylamine |
| DMF | N,N-Dimethylformamide |
| DMSO | Dimethyl sulfoxide |
| eq. | Equivalents |
| ES | Electrospray ionisation |
| h | Hour(s) |
| LCMS | Liquid chromatography-mass spectrometry |
| LRMS | Low resolution mass spectrometry |
| MTBE | Methyl tert-butyl ether |
| MWI | Microwave irradiation |
| rt | room temperature |
| THF | Tetrahydrofuran |
| HATU | 1-[Bis(dimethylamino)methylene]-1H-1,2,3-triazolo[4,5-b]pyridinium 3-oxid hexafluorophosphate, N-[(dimethylamino)-1H-1,2,3-triazolo-[4,5-b]pyridin-1-ylmethylene]-N-methylmethanaminium hexafluorophosphate N-oxide |
| Pd-170 | XPhos Pd(crotyl)Cl |
| SFC | Supercritical fluid chromatography |
| XPhos | 2-Dicyclohexylphosphino-2′,4′,6′ triisopropylbiphenyl |

### Preparatory Examples

### 1A Ethyl 3,5-Dibromo-1H-Pyrazole-4-Carboxylate

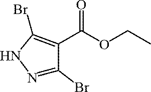

A solution of ethyl 1H-pyrazole-4-carboxylate (5.5 g, 39.3 mmol) in EtOH (60 mL) was cooled to 0° C., then sodium acetate (22.5 g, 271 mmol) in water (90 mL) was added, followed by bromine (8.25 mL, 161 mmol). The reaction was allowed to attain rt, and stirred over the weekend. Sat. aq. Na₂S₂O₃ (100 mL) and EtOAc (50 mL) were added the phases separated, and the aqueous phase extracted with EtOAc (2× 50 mL). The combined organic phases were washed with brine (100 mL), dried (Na₂SO₄) and concentrated under reduced pressure. The crude product was purified by flash chromatography (0-40% MTBE in iso-hexanes) to afford a white solid (10.3 g, 84%). LCMS (method A): m/z 297.3/299.4/301.4 [M+H]⁺ at 1.10 min. ¹H NMR (500 MHz, DMSO-d₆) δ 14.53 (s, 1H), 4.25 (q, J = 7.1 Hz, 2H), 1.29 (t, J = 7.1 Hz, 3H).

### 2A 5-[Tert-Butyl(Dimethyl)Silyl]Oxypentan-2-yl Methanesulfonate

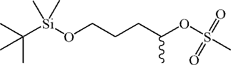 [SMILES: CC(CCCO[Si](C)(C)C(C)(C)C)OS(C)(=O)=O]

A solution of tert-butyldimethylsilyl chloride (1.45 g, 9.62 mmol) in anhydrous CH₂Cl₂ (5 mL) was added to a cooled (0° C.) solution of pentane-1,4-diol (1 g, 9.6 mmol), NEt₃ (3 mL, 22 mmol) and 4-dimethylaminopyridine (250 mg, 2.03 mmol) in anhydrous CH₂Cl₂ (20 mL), allowed to attain rt and stirred for 10 min. The reaction was quenched with 1 M aq. HCl (20 mL), passed through a phase separator, washing with CH₂Cl₂ (3 × 10 mL) and the solvent removed under reduced pressure and the residue taken directly to the next reaction. The crude residue was dissolved in anhydrous CH₂Cl₂ (20 mL), cooled (0° C.), and NEt₃ (3 mL, 22 mmol) and 4-dimethylaminopyridine (250 mg, 2.03 mmol) added. Methanesulfonyl chloride (0.75 mL, 9.69 mmol) was added dropwise and the reaction stirred at rt for 10 min. 1 M aq. HCl (20 mL) was added and the mixture was passed through a phase separator, washing with CH₂Cl₂ (3 × 10 mL) and concentrated under reduced pressure. Purification by flash chromatography (0-50% CH₂Cl₂ in iso-hexanes) afforded a colourless oil (780 mg, 17%). ¹H NMR (500 MHz, CDCl₃) δ 4.90 - 4.80 (m, 1H), 3.68 - 3.59 (m, 2H), 3.00 (s, 3H), 1.80 - 1.55 (m, 4H), 1.43 (d, J = 6.3 Hz, 3H), 0.89 (s, 9H), 0.05 (s, 6H).

### 3A 5-Bromo-2-Methylpentan-2-Ol

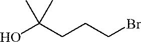

MeMgBr (3.0 M in Et₂O; 3.6 mL, 10.8 mmol) was added dropwise to a cooled (-78° C.) solution of ethyl 4-bromobutanoate (1 g, 5.13 mmol) in anhydrous THF (10 mL). The reaction was allowed to attain rt and stirred overnight, then quenched with sat. aq. NH₄Cl (10 mL). The separated aqueous phase was extracted with EtOAc (2× 10 mL) and the combined organic phases were washed with brine (20 mL), dried (MgSO₄), and concentrated under reduced pressure. Purification by flash chromatography (0-50% MTBE/iso-hexane) afforded a brown oil (604 mg, 62%). ¹H NMR (500 MHz, CDCl₃) δ 3.44 (t, J = 6.7 Hz, 2H), 2.01 - 1.90 (m, 2H), 1.64 - 1.59 (m, 2H), 1.25 (s, 6H).

### 4A 4-Bromo-N-Methoxy-N-Methylbutanamide

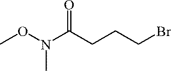

NEt₃ (10.5 mL, 75.1 mmol) was added to a cooled (0° C.) suspension of N,O-dimethyl hydroxylamine hydrochloride (6.71 g, 68.8 mmol) in CH₂Cl₂ (130 mL), followed by dropwise addition of 4-bromobutanoyl chloride (7.24 mL, 62.6 mmol) and additional NEt₃ (10.5 mL, 75.1 mmol). The reaction was stirred for 3 h at rt, the volatiles removed under reduced pressure. The residue was diluted with EtOAc (100 mL), washed successively with 1 M aq. HCl, sat. aq. NaHCO₃ and brine (50 mL each), dried (MgSO₄) and concentrated under reduced pressure to afford a yellow oil (11 g, 80%) which was used without further purification. ¹H NMR (500 MHz, CDCl₃) δ 3.73 (s, 3H), 3.53 (t, J= 6.3 Hz, 2H), 3.20 (s, 3H), 2.64 (t, J = 7.1 Hz, 2H), 2.33 - 2.16 (m, 2H).

### 5A 6-Bromohexan-3-One

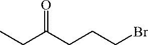

EtMgBr (3.0 M in Et₂O; 3.55 mL, 10.7 mmol) was added to a cooled (-78° C.) solution of intermediate 4A (1.6 g, 7.62 mmol) in anhydrous THF (16 mL). The reaction was warmed to warmed to 0° C. and stirred for 3 h, then quenched with sat. aq. NH₄Cl (50 mL) and extracted with EtOAc (3 × 30 mL). The combined organics were washed with 1 M aq. HCl (50 mL), sat. aq. NaHCO₃ (50 mL) and brine (50 mL), dried (MgSO₄), and concentrated under reduced pressure to afford a yellow oil (1.13 g, 79%) which was used without further purification. ¹H NMR (500 MHz, CDCl₃) δ 3.47 (t, J = 6.4 Hz, 2H), 2.64 (t, J= 7.0 Hz, 2H), 2.47 (q, J= 7.3 Hz, 2H), 2.20 - 2.10 (m, 2H), 1.09 (t, J = 7.3 Hz, 3H).

### 6A Hydroxycyclopentyl) 4-Methylbenzenesulfonate

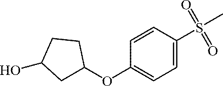 [SMILES: CS(=O)(=O)c1ccc(OC2CCC(O)C2)cc1]

A solution of p-toluenesulfonyl chloride (1 g, 5.25 mmol) in anhydrous CH₂Cl₂ (2 mL) was added dropwise to a cooled (0° C.) solution of cyclopentane-1,3-diol (500 mg, 4.90 mmol), NEt₃ (1.5 mL, 10.8 mmol) and 4-dimethylaminopyridine (121 mg, 0.98 mmol) in anhydrous CH₂Cl₂ (15 mL). The reaction was stirred at rt for 1 h, then partitioned between 1 M aq. HCl (10 mL) and CH₂Cl₂ (10 mL). The separated aqueous phase was extracted with CH₂Cl₂ (2× 10 mL) and the combined organics were washed with 1:1 water/brine (20 mL), dried (MgSO₄), concentrated under reduced pressure and purified by flash chromatography (10-60% EtOAc in iso-hexanes) to afford a colourless gum (302 mg, 22%). ¹H NMR (500 MHz, CDCl₃) δ 7.82 - 7.75 (m, 2H), 7.37 - 7.31 (m, 2H), 5.09 - 5.02 (m, 1H), 4.49 - 4.41 (m, 1H), 2.45 (s, 3H), 2.14 - 2.05 (m, 1H), 2.09 -1.95 (m, 2H), 1.95 - 1.76 (m, 2H), 1.63 - 1.57 (m, 1H), 1.37 (d, J = 3.2 Hz, 1H).

### 7A S-Bromobutyl) Ethanethioate

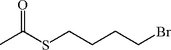

Potassium ethanethioate (574 mg, 5.03 mmol) was added portion-wise to a solution of 1,4-dibromobutane (0.6 mL, 5.03 mmol) in DMF (10 mL). The reaction was stirred at rt for 6 h, diluted with water (20 mL) and extracted with MTBE (3 × 20 mL). The aqueous layer was extracted with MTBE (20 mL) and the combined organics were washed with brine (3 × 40 mL), dried (MgSO₄), and concentrated under reduced pressure. Purification by flash chromatography (0-30% MTBE in iso-hexane) afforded a colourless oil (516 mg, 53%). ¹H NMR (500 MHz, CDCl₃) δ 3.43 (t, J= 6.7 Hz, 2H), 2.92 (t, J= 7.2 Hz, 2H), 2.35 (s, 3H), 2.00 - 1.91 (m, 2H), 1.76 (tt, J = 9.9, 6.2 Hz, 2H).

### 8A 2-(2,2-Dimethyl-1,3-Dioxolan-4-yl)Ethyl Methanesulfonate

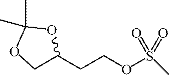

Methanesulfonyl chloride (1.53 mL, 19.8 mmol) was added dropwise to a cooled (0° C.) solution of 2-(2,2-dimethyl-1,3-dioxolan-4-yl)ethanol (2 mL, 14.1 mmol) and NEt₃ (5.11 mL, 36.7 mmol) in CH₂Cl₂ (40 mL). The reaction stirred at rt for 3 h, quenched with 1 M aq. HCl (40 mL), and the organic layer washed with brine (40 mL), passed through a phase separation cartridge and concentrated under reduced pressure to afford a colourless oil (2.81 g, 80%). ¹H NMR (500 MHz, CDCl₃) δ 4.44 - 4.34 (m, 2H), 4.28 - 4.20 (m, 1H), 4.15 - 4.09 (m, 1H), 3.65 - 3.59 (m, 1H), 3.04 (s, 3H), 2.10 - 1.90 (m, 2H), 1.43 (s, 3H), 1.37 (s, 3H).

### 9A Ethyl 3,5-dibromo-1-(4-hydroxybutyl)pyrazole-4-carboxylate

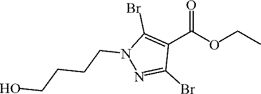 [SMILES: CCOC(=O)c1c(Br)nn(CCCCO)c1Br]

4-Bromobutan-1-ol (0.31 mL, 3.36 mmol) and intermediate 1A (1 g, 3.36 mmol) were added to a solution of K₂CO₃ (1 g, 7.24 mmol) in MeCN (10 mL) and the reaction was heated at 80° C. for 76 h. 4-Bromobutan-1-ol (0.62 mL, 6.72 mmol) was added and heating at 80° C. was continued overnight. The reaction was cooled to rt, filtered, washing with MeCN (2× 20 mL), and the filtrate was concentrated under reduced pressure. Purification by flash chromatography (0-60% EtOAc in iso-hexane) afforded a colourless oil (916 mg, 73%). LCMS (method A): m/z 369.2/371.3/373.3 [M+H]⁺ at 1.65 min.¹H NMR (500 MHz, DMSO-d₆) δ 4.46 (t, J = 5.2 Hz, 1H), 4.26 (q, J = 7.1 Hz, 2H), 4.21 (t, J = 7.1 Hz, 2H), 3.42 - 3.37 (m, 2H), 1.84 - 1.73 (m, 2H), 1.42 - 1.34 (m, 2H), 1.30 (t, J = 7.1 Hz, 3H).

### 10A Ethyl 3,5-dibromo-1-(4-oxopentyl)pyrazole-4-carboxylate

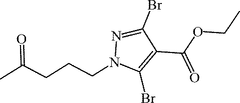 [SMILES: CCOC(=O)c1c(Br)nn(CCCC(C)=O)c1Br]

K₂CO₃ (1.2 g, 8.68 mmol), NaI (1 g, 6.02 mmol) and 5-chloropentan-2-one (0.7 mL, 6.14 mmol) were added to a solution of intermediate 1A (1.2 g, 4.03 mmol) in MeCN (10 mL) and the reaction heated at 60° C. overnight. The reaction was cooled to rt, filtered, washed with MeCN (2× 20 mL), and the filtrate concentrated under reduced pressure. Purification by flash chromatography (0-50% MTBE in iso-hexanes) afforded an orange oil (1.38 g, 83%). LCMS (method A): m/z 381.5/383.5/385.5 [M+H]⁺ at 1.33 min.¹H NMR (500 MHz, CDCl₃) δ 4.35 (q, J = 7.1 Hz, 2H), 4.24 (t, J = 6.7 Hz, 2H), 2.47 (t, J = 6.8 Hz, 2H), 2.15 (s, 3H), 2.14 - 2.07 (m, 2H), 1.39 (t, J = 7.1 Hz, 3H).

### 11A Ethyl 3,5-dibromo-1-(4-oxohexyl)pyrazole-4-carboxylate

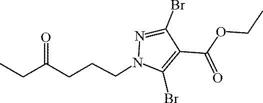 [SMILES: CCOC(=O)c1c(Br)nn(CCCC(=O)CC)c1Br]

K₂CO₃ (1.68 g, 12.2 mmol) and intermediate 5A (1.13 g, 6.33 mmol) were added to a solution of intermediate 1A (1.45 g, 4.87 mmol) in MeCN (10 mL). The reaction was heated at 60° C. for 3 h, cooled to rt, filtered, and washed with MeCN (2× 20 mL). The filtrate was concentrated under reduced pressure and purified by flash chromatography (0-50% MTBE/iso-hexane) to afford a yellow oil (1.78 g, 76%). LCMS (method B): m/z 395.2/397.2/399.2 [M+H]⁺ at 0.66 min.

### 12A Ethyl 3,5-dibromo-1-[5-[tert-butyl(dimethyl)silyl]oxypentan-2-yl]pyrazole-4-carboxylate

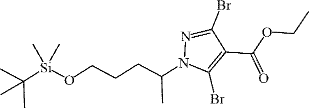 [SMILES: CCOC(=O)c1c(Br)nn(C(C)CCCO[Si](C)(C)C(C)(C)C)c1Br]

K₂CO₃ (750 mg, 5.43 mmol) and intermediate 2A (780 mg, 2.63 mmol) were added to a solution of intermediate 1A (785 mg, 2.63 mmol) in MeCN (10 mL). The reaction was heated at 80° C. overnight, cooled to rt, filtered, and washed with MeCN (2× 20 mL). The filtrate was concentrated under reduced pressure and purified by flash chromatography (0-20%, MTBE in iso-hexanes) to afford a colourless oil (637 mg, 48%). ¹H NMR (500 MHz, CDCl₃) δ 4.72 - 4.61 (m, 1H), 4.35 (q, J = 7.1 Hz, 2H), 3.62 - 3.51 (m, 2H), 2.08 - 1.97 (m, 1H), 1.90 - 1.80 (m, 1H), 1.46 (d, J = 6.6 Hz, 3H), 1.45 - 1.39 (m, 1H), 1.39 (t, J = 7.1 Hz, 3H), 1.36 - 1.24 (m, 1H), 0.88 (s, 9H), 0.03 (s, 6H). LCMS (method A): m/z 497.4/499.4/501.4 [M+H]⁺ at 2.28 min.

### 13A Ethyl 3,5-dibromo-1-(4-hydroxy-4-methylpentyl)pyrazole-4-carboxylate

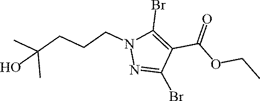 [SMILES: CCOC(=O)c1c(Br)nn(CCCC(C)(C)O)c1Br]

Prepared by an analogous procedure to that described for intermediate 12A with intermediates 1A and 3A. ¹H NMR (500 MHz, CDCl₃) δ 4.35 (q, J = 7.1 Hz, 2H), 4.23 (t, J = 7.3 Hz, 2H), 2.01 - 1.91 (m, 2H), 1.52 - 1.45 (m, 2H), 1.39 (t, J = 7.1 Hz, 3H), 1.23 (s, 6H). LCMS (method A): m/z 379.6/381.6/383.6 [M-OH]⁺ at 1.33 min.

### 14A Ethyl 3,5-dibromo-1-(3-hydroxycyclopentyl)pyrazole-4-carboxylate

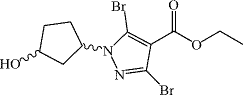 [SMILES: CCOC(=O)c1c(Br)nn(C2CCC(O)C2)c1Br]

Prepared by an analogous procedure to that described for intermediate 12A with intermediates 1A and 6A. LCMS (method A): m/z 381.1/383.1/385.1 [M+H]⁺ at 1.24 min. ¹H NMR (500 MHz, CDCl₃) δ 5.15 - 5.06 (m, 1H), 4.45 - 4.39 (m, 1H), 4.39 -4.32 (m, 2H), 4.01 (d, J = 9.7 Hz, 1H), 2.40 - 2.29 (m, 1H), 2.29 - 2.21 (m, 1H), 2.18 -2.13 (m, 1H), 2.13 - 2.04 (m, 1H), 2.04 - 1.96 (m, 1H), 1.94 - 1.83 (m, 1H), 1.39 (t, J = 7.1 Hz, 3H).

### 15A Ethyl 1-(4-acetylsulfanylbutyl)-3,5-dibromopyrazole-4-carboxylate

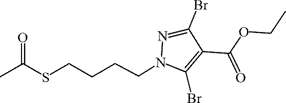 [SMILES: CCOC(=O)c1c(Br)nn(CCCCSC(C)=O)c1Br]

K₂CO₃ (1.41 g, 10.2 mmol) and intermediate 7A (646 mg, 3.06 mmol) were added to a solution of intermediate 1A (760 mg, 2.55 mmol) in MeCN (15 mL). The reaction was heated at 70° C. for 3 days, cooled to rt and filtered and washed with MeCN (2× 20 mL). The filtrate was concentrated under reduced pressure to afford an orange oil (1.06 g, 72%) that was used without further purification. LCMS (method B): m/z 427.2/429.2/431.1 [M+H]⁺ at 0.72 min.

### 16A Ethyl 3,5-dibromo-1-[2-(2,2-dimethyl-1,3-dioxolan-4-yl)ethyl]pyrazole-4-carboxylate

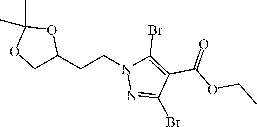 [SMILES: CCOC(=O)c1c(Br)nn(CCC2COC(C)(C)O2)c1Br]

K₂CO₃ (4.45 g, 32.2 mmol) and intermediate 8A (2.81 g, 11.3 mmol) were added to a solution of intermediate 1A (2.4 g, 8.06 mmol) in MeCN (100 mL). The reaction mixture was heated to 70° C. and stirred for 4 h, cooled to rt and filtered, washing with MeCN. The filtrate was concentrated under reduced pressure and purified by flash chromatography (0-60% MTBE/iso-hexane) to afford (2.91 g, 81%) as a colourless oil. LCMS (method A): m/z 425.2/427.2/429.2 [M+H]⁺ at 1.53 min. ¹H NMR (500 MHz, CDCl₃) δ 4.44 - 4.29 (m, 4H), 4.16 - 4.04 (m, 2H), 3.57 (dd, J = 7.9, 6.3 Hz, 1H), 2.21 - 2.00 (m, 2H), 1.46 - 1.34 (m, 9H).

### 17A Ethyl 3,5-dibromo-1-(3,4-dihydroxybutyl)pyrazole-4-carboxylate

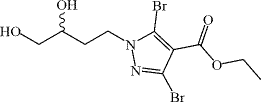 [SMILES: CCOC(=O)c1c(Br)nn(CCC(O)CO)c1Br]

A solution of intermediate 16A (2.91 g, 6.83 mmol) in AcOH (20 mL) and water (10 mL) was heated at 110° C. for 2 h, cooled to rt and concentrated under reduced pressure. The residue was dissolved in CH₂Cl₂ (30 mL), and washed with sat. aq. NaHCO₃ (30 mL) and brine (30 mL), passed through a phase separation cartridge and the solvent removed under reduced pressure to afford a colourless oil (2.80 g, 85%) which was used without further purification. LCMS (method B): m/z 385.4/387.5/389.5 [M+H]⁺ at 0.50 min.¹H NMR (500 MHz, CDCl₃) δ 4.50 - 4.34 (m, 4H), 3.79 - 3.65 (m, 1H), 3.55 -3.49 (m, 1H), 2.12 (d, J= 2.4 Hz, 1H), 2.10 - 1.97 (m, 1H), 1.97 - 1.91 (m, 1H), 1.91 (m, 1H), 1.41 (td, J = 7.1, 2.9 Hz, 3H).

### 18A Ethyl 3,5-dibromo-1-[4-[tert-butyl(dimethyl)silylfoxy-3-hydroxybutyl]pyrazole-4-carboxylate

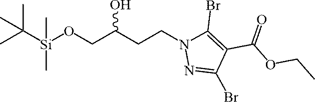 [SMILES: CCOC(=O)c1c(Br)nn(CCC(O)CO[Si](C)(C)C(C)(C)C)c1Br]

A solution of intermediate 17A (2.8 g, 6.15 mmol) in CH₂Cl₂ (10 mL) was added to a solution of tert-butyldimethylsilyl chloride (1.11 g, 7.39 mmol) and imidazole (1.05 g, 15.4 mmol) in CH₂Cl₂ (40 mL) and stirred at rt for 72 h. The reaction mixture was filtered, washing with CH₂Cl₂ (2× 20 mL), the filtrate concentrated under reduced pressure and purified by flash chromatography (0-50% MTBE in heptane) to afford a light yellow oil (1.91 g, 53%). LCMS (method A): m/z 499.3/501.3/503.3 [M+H]⁺ at 1.96 min.¹H NMR (500 MHz, CDCl₃) δ 4.43 - 4.36 (m, 4H), 3.71 - 3.61 (m, 2H), 3.51 - 3.40 (m, 1H), 2.54 (s, 1H), 2.07 - 1.99 (m, 1H), 1.97 - 1.86 (m, 1H), 1.42 (d, J = 7.2 Hz, 3H), 0.92 (s, 9H), 0.09 (s, 6H).

### 19A Ethyl 3,5-dibromo-1-[4-[tert-butyl(dimethyl)silyl]oxy-3-oxobutyl]pyrazole-4-carboxylate

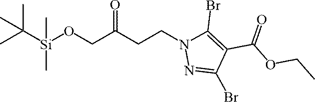 [SMILES: CCOC(=O)c1c(Br)nn(CCC(=O)CO[Si](C)(C)C(C)(C)C)c1Br]

Dess-Martin periodinane (540 mg, 1.27 mmol) was added to a solution of intermediate 18A (0.5 g, 0.85 mmol) in CH₂Cl₂ (10 mL) and the reaction mixture was stirred at rt for 2 h. The reaction was quenched with 10% aq. Na₂S₂O₅ (20 mL) and washed with sat. aq. NaHCO₃ (20 mL). This workup process was repeated twice more, then the organics were washed with brine (20 mL), passed through a phase separation cartridge, and concentrated under reduced pressure to afford as a colourless oil (414 mg, 92% yield). LCMS (method A): m/z 497.2/499.2/501.2 [M+H]⁺ at 2.00 min.¹H NMR (500 MHz, CDCl₃) δ 4.48 (t, J = 7.0 Hz, 2H), 4.37 (d, J= 7.1 Hz, 2H), 4.22 (s, 2H), 3.17 (t, J = 7.0 Hz, 2H), 1.41 (t, J = 7.1 Hz, 3H), 0.94 (s, 9H), 0.11 (s, 6H).

### 20A Ethyl 3,5-dibromo-1-[4-[tert-butyl(dimethyl)silyl]oxy-3,3-difluorobutyl]pyrazole-4-carboxylate

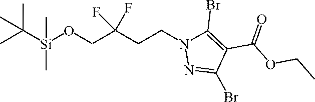 [SMILES: CCOC(=O)c1c(Br)nn(CCC(F)(F)CO[Si](C)(C)C(C)(C)C)c1Br]

Diethylaminosulfur trifluoride (2.78 mL, 21.07 mmol) was added to a solution of intermediate 19A (2.1 g, 4.21 mmol) in CH₂Cl₂ (100 mL) and stirred for 24 h at rt. The reaction was cooled to 0° C., quenched with sat. aq. NaHCO₃ solution (100 mL) and the layers were separated. The organic fraction was washed with brine (50 mL), passed through a phase separation cartridge and concentrated under reduced pressure. Purification by flash chromatography (0-100% MTBE/iso-hexanes) afforded a colourless oil (801 mg, 20%). LCMS (method A): m/z 519.3/521.2/523.2 [M+H]⁺ at 2.17 min.

### 40A Ethyl 3,5-dibromo-1-[4-[tert-butyl(dimethyl)silyl]oxy-3-morpholin-4-ylbutyl]pyrazole-4-carboxylate

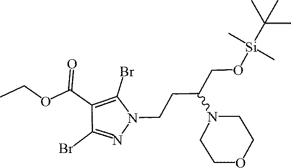 [SMILES: CCOC(=O)c1c(Br)nn(CCC(CO[Si](C)(C)C(C)(C)C)N2CCOCC2)c1Br]

Morpholine (395 µL, 4.52 mmol) was added to a solution of intermediate 19A (900 mg, 1.81 mmol) in CH₂Cl₂ (10 mL) and the reaction mixture was stirred for 20 minutes at rt, after which time sodium triacetoxyborohydride (1.53 g, 7.22 mmol) was added and the mixture was stirred at rt for 8 days. The reaction mixture was quenched with sat. aq. NaHCO₃ (20 mL), and the aqueous layer was extracted with EtOAc (30 mL). The organics were washed with brine (20 mL), passed through a phase separation cartridge and concentrated in vacuo to give a crude residue that was purified by column chromatography (0-100% MTBE in isohexane, then by 10% MeOH in MTBE) to give a colourless oil (183 mg, 18%). LCMS (method A): m/z 568.2/570.2/572.2 [M+H]⁺ at 1.32 min.

### 21A Ethyl 3, 5-dibromo-1-(5-hydroxypentan-2-yl)pyrazole-4-carboxylate

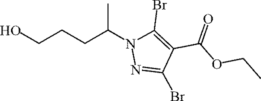 [SMILES: CCOC(=O)c1c(Br)nn(C(C)CCCO)c1Br]

Tetrabutylammonium fluoride (1 M in THF; 1.4 mL, 1.40 mmol) was added to a solution of intermediate 12A (635 mg, 1.27 mmol) in anhydrous THF (5 mL). The reaction was stirred at rt for 10 min, and then sat. aq. NH₄Cl (10 mL) and EtOAc (20 mL) were added. The separated aqueous phase was extracted with EtOAc (2× 10 mL), the combined organics washed with brine (20 mL), dried (MgSO₄) and concentrated under reduced pressure. Purification by flash chromatography (10-70% MTBE in iso-hexanes) afforded a pale yellow gum (449 mg, 91%). LCMS (method A): m/z 383.2/385.2/387.2 [M+H]⁺ at 1.27 min. ¹H NMR (500 MHz, CDCl₃) δ 4.73 - 4.63 (m, 1H), 4.35 (q, J = 7.1 Hz, 2H), 3.67 - 3.54 (m, 2H), 2.14 - 2.03 (m, 1H), 1.93 - 1.82 (m, 1H), 1.54 - 1.48 (m, 1H), 1.47 (d, J = 6.7 Hz, 3H), 1.39 (t, J = 7.1 Hz, 3H), 1.37 - 1.31 (m, 2H).

### 21B Ethyl 3,5-dibromo-1-(3,3-difluoro-4-hydroxybutyl)pyrazole-4-carboxylate

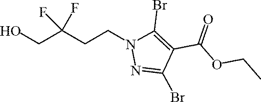 [SMILES: CCOC(=O)c1c(Br)nn(CCC(F)(F)CO)c1Br]

Tetrabutylammonium fluoride (1 M in THF; 1.7 mL, 1.7 mmol) was added to a solution of intermediate 20A (221 mg, 0.42 mmol) in anhydrous THF (5 mL). The reaction mixture was stirred at rt for 3 h, diluted with EtOAc (20 mL) and quenched with sat. aq. NH₄Cl (20 mL). The aqueous layer was further extracted with EtOAc (2× 20 mL), and the combined organics were washed with brine (20 mL), dried (MgSO₄), and concentrated under reduced pressure. Purification by flash chromatography (0-100% MTBE in iso-hexane) afforded (105 mg, 59%) as a colourless solid. LCMS (method A): m/z 405.5/407.5/409.5 [M+H]⁺ at 1.29 min.

### 21C Ethyl 3,5-dibromo-1-(4-hydroxy-3-morpholin-4-ylbutyl)pyrazole-4-carboxylate

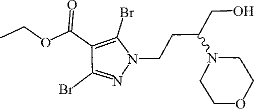 [SMILES: CCOC(=O)c1c(Br)nn(CCC(CO)N2CCOCC2)c1Br]

Prepared by an analogous procedure to that described for intermediate 21A. LCMS (method A): m/z 454.2/456.1/458.1 [M+H]⁺ at 0.67 min.

### 22A Ethyl 3,5-dibromo-1-(4-hydroxypentyl)pyrazole-4-carboxylate

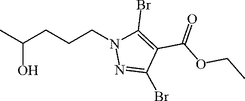 [SMILES: CCOC(=O)c1c(Br)nn(CCCC(C)O)c1Br]

NaBH₄ (408 mg, 10.8 mmol) was added to a cooled (0° C.) solution of intermediate 10A (4.08 g, 10.7 mmol) in EtOH (30 mL). The reaction was warmed to rt over 10 minutes, stirred for 1 h and concentrated under reduced pressure. The residue was partitioned between 1 M aq. HCl (50 mL) and EtOAc (50 mL), the separated aqueous phase extracted with EtOAc (2× 10 mL). The combined organics were washed with brine (50 mL), dried (MgSO₄), and concentrated under reduced pressure to afford a yellow oil (3.84 g, 93%). LCMS (method A): m/z 383.2/385.2/387.2 [M+H]⁺ at 1.25 min. ¹H NMR (500 MHz, CDCl₃) δ 4.35 (q, J = 7.1 Hz, 2H), 4.30 - 4.17 (m, 2H), 3.88 - 3.78 (m, 1H), 2.07 - 1.86 (m, 2H), 1.51 - 1.42 (m, 2H), 1.41 - 1.37 (m, 1H), 1.39 (t, J = 7.1 Hz, 3H), 1.20 (d, J = 6.2 Hz, 3H).

### 22B Ethyl 3,5-dibromo-1-(4-hydroxyhexyl)pyrazole-4-carboxylate

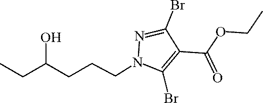 [SMILES: CCOC(=O)c1c(Br)nn(CCCC(O)CC)c1Br]

Prepared by an analogous procedure to that described for intermediate 22A with intermediate 11A. LCMS (method B): m/z 397.2/399.5/401.4 [M+H]⁺ at 0.64 min.

### 23A Ethyl 2-Bromo-5-Methyl-5,6,7,8-Tetrahydropyrazolo[5,1-b][1,3]Oxazepine-3-Carboxylate

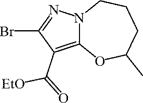 [SMILES: CCOC(=O)c1c(Br)nn2c1OC(C)CCC2]

Sodium hydride (270 mg, 6.75 mmol) was added to a solution of intermediate 22A (1.26 g, 3.28 mmol) in dry THF (10 mL) at 0° C. The reaction mixture was allowed to attain rt and stirred for 3 days. Water (10 mL) and CH₂Cl₂ (20 mL) were added, and the separated aqueous fraction extracted with CH₂Cl₂ (3 × 10 mL). The combined organics were washed with brine (20 mL), dried (MgSO₄) and concentrated under reduced pressure. Purification by flash chromatography (0-50% MTBE in iso-hexanes) afforded a white solid (664 mg, 66%). LCMS (method A): m/z 303.2/305.2 [M+H]⁺ at 1.28 min. ¹H NMR (500 MHz, CDCl₃) δ 4.37 - 4.23 (m, 3H), 4.10 - 4.01 (m, 2H), 2.09 (ddd, J = 14.6, 5.1, 3.2 Hz, 1H), 2.05 - 1.94 (m, 1H), 1.94 - 1.87 (m, 1H), 1.79 - 1.69 (m, 1H), 1.49 (d, J = 6.3 Hz, 3H), 1.36 (t, J = 7.1 Hz, 3H).

The following intermediate compounds listed in Table 1 were prepared by the same general procedure.

TABLE 1

Preparatory Examples prepared via general cyclisation procedure

| | | | | |
|---|---|---|---|---|
| Preparatory Example | Name | Structure | ¹H NMR (500 MHz) δ | LCMS (ES+) m/z |
| 23B | Ethyl 2-bromo-5,6,7,8-tetrahydropyrazolo[5,1-b][1,3]oxazepine-3-carboxylate | 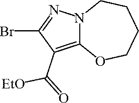 [SMILES: CCOC(=O)c1c(Br)nn2c1OCCCC2] | (DMSO-d₆) 4.21 - 4.14 (m, 6H), 2.02 - 1.96 (m, 2H), 1.83 - 1.75 (m, 2H), 1.24 (t, J = 7.1 Hz, 3H). | (method A) 289.2/291.2 [M+H]⁺ at 1.14 min |
| 23C | Ethyl 2-bromo-5-ethyl-5,6,7,8-tetrahydropyrazolo[5,1-b][1,3]oxazepine-3-carboxylate | 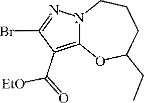 [SMILES: CCOC(=O)c1c(Br)nn2c1OC(CC)CCC2] | Not available | (method B) 317.2/319.2 [M+H]⁺ at 0.66 min |
| 23D | Ethyl 2-bromo-5,5-dimethyl-7,8-dihydro-6H-pyrazolo[5,1-b][1,3]oxazepine-3-carboxylate | 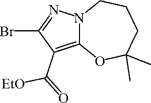 [SMILES: CCOC(=O)c1c(Br)nn2c1OC(C)(C)CCC2] | (CDCl₃) 4.30 (q, J = 7.1 Hz, 2H), 4.19 - 4.13 (m, 2H), 2.03 - 1.97 (m, 2H), 1.91 - 1.83 (m, 2H), 1.39 (s, 6H), 1.37 (t, J = 7.1 Hz, 3H). | (method A) 317.2/319.2 [M+H]⁺ at 1.35 min |
| 23E | Ethyl 2-bromo-8-methyl-5,6,7,8-tetrahydropyrazolo[5,1-b][1,3]oxazepine-3-carboxylate | 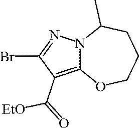 [SMILES: CCOC(=O)c1c(Br)nn2c1OCCCC2C] | (CDCl₃) 4.65 - 4.57 (m, 1H), 4.43 - 4.36 (m, 1H), 4.36 -4.24 (m, 2H), 4.05 - 3.96 (m, 1H), 2.37 - 2.25 (m, 1H), 2.04 - 1.92 (m, 2H), 1.92 - 1.82 (m, 1H), 1.49 (d, J = 7.0 Hz, 3H), 1.35 (t, J = 7.1 Hz, 3H). | (method A) 303.2/305.2 [M+H]⁺ at 1.27 min |
| 23F | Ethyl 2-bromo-6,6-difluoro-7,8-dihydro-5H-pyrazolo[5,1-b][1,3]oxazepine-3-carboxylate | 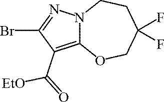 [SMILES: CCOC(=O)c1c(Br)nn2c1OCC(F)(F)CC2] | Not available | (method A) 325.1/327.1 [M+H]⁺ at 1.28 min |
| 23G | Ethyl 4-bromo-7-oxa-2,3-diazatricyclo[6.2.1.0 ^{2,6}]undeca-3,5-diene- 5-carboxylate | 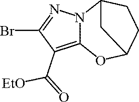 [SMILES: CCOC(=O)c1c(Br)nn2c1OC1CCC2C1] | (CDCl₃) δ 5.23 - 5.18 (m, 1H), 4.72 - 4.69 (m, 1H), 4.29 (q, J = 7.1 Hz, 2H), 2.41 -2.06 (m, 4H), 2.03 - 1.92 (m, 2H), 1.41 - 1.27 (m, 3H). | (method A) 303.2/305.2 [M+H]⁺ at 1.09 min |
| 23H | Ethyl 2-bromo-6-morpholin-4-yl-5,6,7,8-tetrahydropyrazolo[5,1-b][1,3]oxazepine-3-carboxylate | 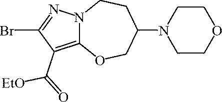 [SMILES: CCOC(=O)c1c(Br)nn2c1OCC(N1CCOCC1)CC2] | Not available | (method A) 374.6/376.7 [M+H]⁺ at 0.20 min |

### 24A Ethyl 2-bromo-5,6,7,8-tetrahydropyrazolo[5,1-b][1,3]thiazepine-3-carboxylate

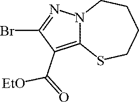 [SMILES: CCOC(=O)c1c(Br)nn2c1SCCCC2]

Intermediate 15A (1.06 g, 2.48 mmol) was added to a suspension of K₂CO₃ (1.71 g, 12.4 mmol) in water (10 mL) and EtOH (10 mL). The reaction mixture was heated at 70° C. for 2 h, cooled to rt and concentrated under reduced pressure. Water (30 mL) and EtOAc (30 mL) were added, and the separated aqueous layer was extracted with EtOAc (30 mL). The combined organics were washed with brine (30 mL), dried (MgSO₄) and concentrated under reduced pressure to afford a colourless oil (673 mg, 86%). LCMS (method B): m/z 305.2/307.2 [M+H]⁺ at 0.64 min.

### 25A Ethyl 2-phenyl-5,6, 7, 8-tetrahydropyrazolo[5,1-b][1,3]oxazepine-3-carboxylate

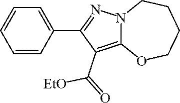 [SMILES: CCOC(=O)c1c(-c2ccccc2)nn2c1OCCCC2]

A reaction vessel was charged with intermediate 23B (140 mg, 0.484 mmol), phenyl boronic acid (118 mg, 0.97 mmol), K₃PO₄ (124 mg, 0.583 mmol), XPhos (12 mg, 0.025 mmol) and XPhos Pd(crotyl)Cl (Pd-170; 18 mg, 0.026 mmol). The reaction vessel was evacuated, filled with N₂, THF:water (~4:1; 5 mL) added, the mixture sparged with N₂ and heated to 65° C. overnight. The reaction was cooled to rt, diluted with EtOAc (25 mL), washed with brine (3 × 25 mL), dried (Na₂SO₄) and the solvent removed under reduced pressure. Purification by flash chromatography (0-70% EtOAc in iso-hexanes) afforded a white solid (186 mg, 94%). LCMS (method A): m/z 287.3 [M+H]⁺ at 1.31 min. ¹H NMR (500 MHz, DMSO-d₆) δ 7.92 - 7.85 (m, 1H), 7.60 - 7.52 (m, 2H), 7.43 -7.29 (m, 2H), 4.26 - 4.20 (m, 2H), 4.22 - 4.16 (m, 2H), 4.11 (q, J = 7.1 Hz, 2H), 2.07 -1.99 (m, 2H), 1.88 - 1.80 (m, 2H), 1.14 (t, J = 7.1 Hz, 3H).

The following preparatory examples were prepared in an analogous manner to intermediate 25A. For preparatory examples 25E, 25F, and 25G, THF was used as the reaction solvent instead of THF:water (~4:1).

| | | | | |
|---|---|---|---|---|
| Preparatory Example | Name | Structure | ¹H NMR δ (500 MHz) | LCMS (ES+) m/z |
| 25B | Ethyl 2-(2,6-difluoropyridin-3-yl)-5,6,7,8-tetrahydropyrazolo[ 5,1-b][1,3]oxazepine-3-carboxylate | 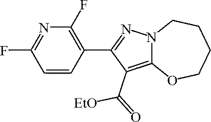 [SMILES: CCOC(=O)c1c(-c2ccc(F)nc2F)nn2c1OCCCC2] | (DMSO-d₆) 8.24 - 8.14 (m, 1H), 7.24 (dd, J = 8.1, 2.5 Hz, 1H), 4.29 - 4.24 (m, 2H), 4.26 - 4.20 (m, 2H), 4.06 (q, J = 7.1 Hz, 2H), 2.08 - 2.00 (m, 2H), 1.90 -1.81 (m, 2H), 1.07 (t, J = 7.1 Hz, 3H). | (method A) 324.3 [M+H]⁺ at 1.25 min |
| 25C | Ethyl 2-(2,6-difluoropyridin-3-yl)-5-methyl-5,6,7,8-tetrahydropyrazolo[ 5,1- | 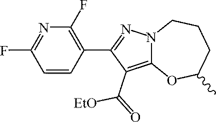 [SMILES: CCOC(=O)c1c(-c2ccc(F)nc2F)nn2c1OC(C)CCC2] | (CDCl₃) 8.03 - 7.95 (m, 1H), 6.86 (dd, J = 8.0, 2.8 Hz, 1H), 4.43 - 4.35 (m, 1H), 4.24 - 4.10 (m, 4H), 2.17 - 2.09 (m, 1H), 2.12 -2.02 (m, 1H), 2.02 - 1.91 (m, 1H), 1.88 - 1.76 (m, | (method A) 338.5 [M+H]⁺ at 1.38 min |
| | b][1,3]oxazepine-3-carboxylate | | 1H), 1.53 (d, J = 6.4 Hz, 3H), 1.21 (t, J = 7.2 Hz, 3H). | |
| 25D | Ethyl 2-(2-fluorophenyl)-8-methyl-5,6,7,8-tetrahydropyrazolo[ 5,1-b][1,3]oxazepine-3-carboxylate | 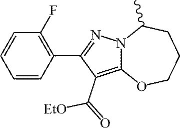 [SMILES: CCOC(=O)c1c(-c2ccccc2F)nn2c1OCCCC2C] | (CDC1₃) 7.48 (td, J = 7.4, 1.8 Hz, 1H), 7.45 - 7.31 (m, 1H), 7.21 - 7.14 (m, 1H), 7.12 - 7.04 (m, 1H), 4.74 -4.66 (m, 1H), 4.51 - 4.43 (m, 1H), 4.22 - 4.09 (m, 2H), 4.08 - 4.00 (m, 1H), 2.42 - 2.31 (m, 1H), 2.09 -1.89 (m, 3H), 1.54 (d, J = 7.0 Hz, 3H), 1.12 (t, J = 7.1 Hz, 3H). | (method A) 319.3 [M+H]⁺ at 1.40 min |
| 25E | Ethyl 2-(2-fluorophenyl)-5-methyl-5,6,7,8-tetrahydropyrazolo[ 5,1-b][1,3]oxazepine-3-carboxylate | 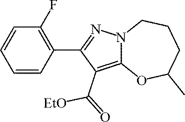 [SMILES: CCOC(=O)c1c(-c2ccccc2F)nn2c1OC(C)CCC2] | (CDC1₃) 7.46 (td, J = 7.4, 1.9 Hz, 1H), 7.39 - 7.31 (m, 1H), 7.17 (td, J = 7.5, 1.2 Hz, 1H), 7.13 - 7.05 (m, 1H), 4.45 - 4.37 (m, 1H), 4.25 - 4.09 (m, 4H), 2.15 -2.09 (m, 1H), 2.08 - 2.01 (m, 1H), 2.01 - 1.90 (m, 1H), 1.89 - 1.77 (m, 1H), 1.53 (d, J = 6.4 Hz, 3H), 1.18 (t, J = 7.1 Hz, 3H). | (method A) 319.7 [M+H]⁺ at 1.40 min |
| 25F | Ethyl 2-(2,4-difluorophenyl)-5-methyl-5,6,7,8-tetrahydropyrazolo[ 5,1-b][1,3]oxazepine-3-carboxylate | 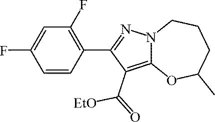 [SMILES: CCOC(=O)c1c(-c2ccc(F)cc2F)nn2c1OC(C)CCC2] | Not available | (method B) 337.5 [M+H]⁺ at 0.68 min |
| 25G | Ethyl 2-(2-fluorophenyl)-5,5-dimethyl-7,8-dihydro-6H-pyrazolo[5,1-b][1,3]oxazepine-3-carboxylate | 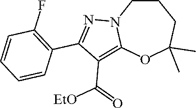 [SMILES: CCOC(=O)c1c(-c2ccccc2F)nn2c1OC(C)(C)CCC2] | (CDC1₃) 7.50 - 7.43 (m, 1H), 7.39 - 7.31 (m, 1H), 7.20 - 7.13 (m, 1H), 7.12 -7.05 (m, 1H), 4.27 - 4.22 (m, 2H), 4.17 (q, J = 7.1 Hz, 2H), 2.07 - 2.01 (m, 2H), 1.97 - 1.89 (m, 2H), 1.44 (s, 6H), 1.19 (t, J = 7.1 Hz, 3H). | (method A) 333.7 [M+H]⁺ at 1.46 min |
| 25H | Ethyl 4-(2-Fluorophenyl)-7-oxa-2,3-diazatricyclo[6.2.1. 02,6]undeca-3,5-diene-5-carboxylate | 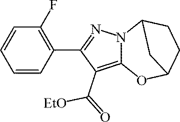 [SMILES: CCOC(=O)c1c(-c2ccccc2F)nn2c1OC1CCC2C1] | (CDC1₃) 7.49 - 7.42 (m, 1H), 7.39 - 7.31 (m, 1H), 7.20 - 7.11 (m, 1H), 7.11 -7.04 (m, 1H), 5.27 - 5.22 (m, 1H), 4.81 - 4.77 (m, 1H), 4.13 (q, J = 7.1 Hz, 2H), 2.48 - 2.39 (m, 2H), 2.35 - 2.24 (m, 1H), 2.23 -2.13 (m, 1H), 2.06 - 1.98 (m, 2H), 1.10 (t, J = 7.1 Hz, 3H). | (method A) 317.3 [M+H]⁺ at 1.23 min |

### 26A Ethyl 2-(5-methylpyridin-3-yl)-5,6, 7,8-tetrahydropyrazolo[5,1-b][1,3]oxazepine-3-carboxylate

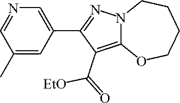 [SMILES: CCOC(=O)c1c(-c2cncc(C)c2)nn2c1OCCCC2]

A reaction vessel was charged with intermediate 23B (140 mg, 0.484 mmol), 5-methylpyridine-3-boronic acid (133 mg, 0.970 mmol), K₃PO₄ (124 mg, 0.583 mmol), XPhos (12 mg, 0.025 mmol) and Pd-170 (18 mg, 0.026 mmol). The reaction vessel was evacuated, filled with N₂, THF:water (~4:1; 5 mL) added, the mixture sparged with N₂ and heated to 65° C. overnight. Further Pd-170 (18 mg, 0.026 mmol) and 5-methylpyridine-3-boronic acid (133 mg, 0.970 mmol) were added to the reaction, which was sparged with N₂ and heated to 65° C. overnight. The reaction was cooled to rt, diluted with MeOH (25 mL) and passed through a silica-propylsulfonic acid solid phase extraction cartridge (Isolute SCX-2, 2 g), which was washed with MeOH (100 mL), followed by product elution with 0.7 M NH₃ in MeOH (100 mL). The solvent was removed under reduced pressure to afford an orange oil (154 mg, 84%) which was used without further purification. LCMS (method A): m/z 302.3 [M+H]⁺ at 0.72 min. ¹H NMR (500 MHz, DMSO-d₆) 8.53 (d, J = 2.1 Hz, 1H), 8.39 (d, J = 1.4 Hz, 1H), 7.78 -7.75 (m, 1H), 4.27 - 4.23 (m, 2H), 4.22 - 4.19 (m, 2H), 4.12 (q, J = 7.1 Hz, 2H), 2.35 -2.30 (m, 3H), 2.05 - 2.01 (m, 2H), 1.90 - 1.79 (m, 2H), 1.14 (t, J = 7.1 Hz, 3H).

### 27A Ethyl 5-ethyl-2-(2-fluorophenyl)-5,6,7,8-tetrahydropyrazolo[5,1-b][1,3]oxazepine-3-carboxylate

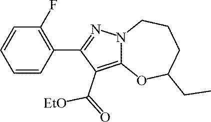 [SMILES: CCOC(=O)c1c(-c2ccccc2F)nn2c1OC(CC)CCC2]

Prepared by an analogous procedure to that described for intermediate 25B from intermediate 23C (269 mg, 0.85 mmol), 2-fluorophenylboronic acid (237 mg, 1.696 mmol), Pd-170 (43 mg, 0.064 mmol), XPhos (40 mg, 0.085 mmol) and K₃PO₄ (234 mg, 1.103 mmol) in THF:water (3:2; 10 mL) with heating at 70° C. for 18 h. LCMS (method A): m/z 333.3 [M+H]⁺ at 0.68 min.

### 28A 2-Ethyl-5-(4,4,5,5-Tetramethyl-1,3,2-Dioxaborolan-2-yl)Pyridine

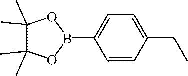 [SMILES: CCc1ccc(B2OC(C)(C)C(C)(C)O2)cc1]

Potassium acetate (633 mg, 6.45 mmol) was added to a suspension of bis(pinacolato)diboron (601 mg, 2.365 mmol) and 5-bromo-2-ethylpyridine (400 mg, 2.150 mmol) in dioxane (3.3 mL). The reaction mixture was sparged with N₂, Pd(dppf)C1₂ (118 mg, 0.161 mmol) added, and heated at 100° C. for 1 h. The reaction was cooled to rt, diluted with EtOAc (30 mL) and filtered through Celite, washing with EtOAc (40 mL). The filtrate was concentrated under reduced pressure to afford a red oil (501 mg, 100% yield) which was used without further purification. ¹H NMR (500 MHz, DMSO-d₆) δ 8.87 (s, 1H), 8.01 (dd, J = 7.7, 1.7 Hz, 1H), 7.19 (d, J = 7.7 Hz, 1H), 2.86 (q, J = 7.6 Hz, 2H), 1.34 (s, 12H), 1.30 (t, J = 7.6 Hz, 3H).

### 29A Ethyl 6,6-Difluoro-2-(2-Fluorophenyl)-7,8-Dihydro-5H-Pyrazolo[5,1-b][1,3]Oxazepine-3-Carboxylate

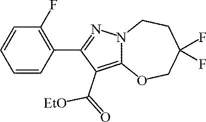 [SMILES: CCOC(=O)c1c(-c2ccccc2F)nn2c1OCC(F)(F)CC2]

A solution of intermediate 23F (41 mg, 0.126 mmol), 2-fluorophenylboronic acid (35 mg, 0.252 mmol), K₃PO₄ (107 mg, 0.504 mmol) and XPhos (12 mg, 0.025 mmol) in 3:2 THF:water (10 mL) was sparged with N₂, Pd-170 (8.5 mg, 0.013 mmol) added and the reaction heated at 70° C. for 3 h. The reaction was cooled to rt, diluted with EtOAc (10 mL), washed with brine and sat. aq. NaHCO₃, dried (MgSO₄) and the solvent removed under reduced pressure. Purification by flash chromatography (0-100% MTBE in heptane) afforded a colourless oil (32 mg, 75%). LCMS (method A): m/z 341.3 [M+H]⁺ at 1.42 min.

The following preparatory examples were prepared in an analogous manner to intermediate 28A. Preparatory examples 29C and 42B were prepared from the corresponding pinacol ester intermediate 28A.

| | | | |
|---|---|---|---|
| Preparatory Example | Name | Structure | LCMS (ES+) m/z |
| 29B | Ethyl 6,6-difluoro-2-(6-fluoropyridin-3-yl)-7,8-dihydro-5H-pyrazolo[5,1-b][1,3]oxazepine-3-carboxylate | 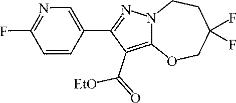 [SMILES: CCOC(=O)c1c(-c2ccc(F)nc2)nn2c1OCC(F)(F)CC2] | (method A) 342.3 [M+H]⁺ at 1.29 min |
| 29C | Ethyl 2-(6-ethylpyridin-3-yl)-6,6-difluoro-7,8-dihydro-5H-pyrazolo[5,1-b][1,3]oxazepine-3-carboxylate | 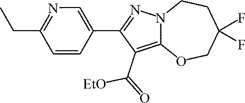 [SMILES: CCOC(=O)c1c(-c2ccc(CC)nc2)nn2c1OCC(F)(F)CC2] | (method A) 352.7 [M+H]⁺ at 0.86 min |
| 41A | Ethyl 2-(2-fluorophenyl)-6-morpholin-4-yl-5,6,7,8-tetrahydropyrazo1o[5, 1-b][1,3]oxazepine-3-carboxylate | 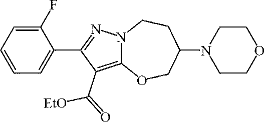 [SMILES: CCOC(=O)c1c(-c2ccccc2F)nn2c1OCC(N1CCOCC1)CC2] | (method A): 390.0 [M+H]⁺ at 0.70 min |
| 42A | Ethyl 4-(6-fluoropyridin-3-yl)-7-oxa-2,3-diazatricyclo[6.2.1.02, 6]undeca-3,5-diene-5-carboxylate | 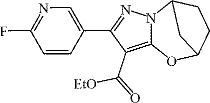 [SMILES: CCOC(=O)c1c(-c2ccc(F)nc2)nn2c1OC1CCC2C1] | (method A): 318.3 [M+H]⁺ at 1.12 min |
| 42B | Ethyl 4-(6-ethylpyridin-3-yl)-7-oxa-2,3-diazatricyclo[6.2.1.02, 6]undeca-3,5-diene-5-carboxylate | 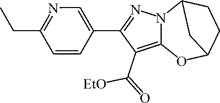 [SMILES: CCOC(=O)c1c(-c2ccc(CC)nc2)nn2c1OC1CCC2C1] | (method A): 328.4 [M+H]⁺ at 0.71 min |

### 30A Ethyl 2-(2-fluorophenyl)-5, 6,7, 8-tetrahydropyrazolo[5,1-b][1,3]oxazepine-3-carboxylate

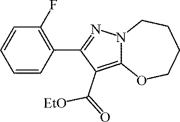 [SMILES: CCOC(=O)c1c(-c2ccccc2F)nn2c1OCCCC2]

A reaction vessel was charged with intermediate 23B (187 mg, 0.613 mmol), 2-fluorophenylboronic acid (172 mg, 1.227 mmol), K₃PO₄ (156 mg, 0.740 mmol), and XPhos Pd G2 (25.8 mg, 0.033 mmol). The reaction vessel was evacuated, filled with N₂, THF (4 mL) and water (1 mL) added, sparged with N₂ and heated at 65° C. overnight. The cooled reaction was partitioned between EtOAc (10 mL) and water (10 mL), and separated aqueous phase extracted with EtOAc (3× 10 mL) and the organic layers were combined, washed with brine (20 mL), dried (Na₂SO₄) and concentrated under reduced pressure. Purification by flash chromatography (0-80% EtOAc in iso-hexanes) afforded a white solid (186 mg, 94%). LCMS (method A): m/z 305.3 [M+H]⁺ at 1.28 min. ¹H NMR (500 MHz, DMSO-d₆) δ 7.48 - 7.39 (m, 2H), 7.26 - 7.18 (m, 2H), 4.27 - 4.22 (m, 2H), 4.24 - 4.18 (m, 2H), 4.03 (q, J = 7.1 Hz, 2H), 2.07 - 1.99 (m, 2H), 1.89 - 1.81 (m, 2H), 1.04 (t, J = 7.1 Hz, 3H).

### 31A Ethyl 5-ethyl-2-(6-methylpyridin-3-yl)-5,6,7,8-tetrahydropyrazolo[5,1-b][1,3]oxazepine-3-carboxylate

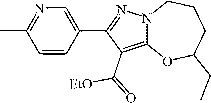 [SMILES: CCOC(=O)c1c(-c2ccc(C)nc2)nn2c1OC(CC)CCC2]

A reaction vessel was charged with intermediate 23C (269 mg, 0.848 mmol), 2-methyl-5-(4,4,5,5-tetramethyl-1,3,2-dioxaborolan-2-yl)pyridine (372 mg, 1.696 mmol), XPhos (40 mg, 0.085 mmol) and K₃PO₄ (234 mg, 1.103 mmol). THF (6 mL) and water (4 mL) were added, the mixture sparged with N₂, then XPhos Pd G2 (50 mg, 0.064 mmol) was added and the reaction heated at 70° C. for 18 h. Analogous workup to that described for intermediate followed by purification by flash chromatography (0-100% MTBE in iso-hexanes) afforded a white solid (186 mg, 94%). LCMS (method B): m/z 330.3 [M+H]⁺ at 0.46 min.

### 32A Ethyl 2-(2-fluorophenyl)-5,6,7,8-tetrahydropyrazolo[5,1-b][1,3]thiazepine-3-carboxylate

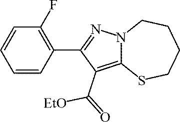 [SMILES: CCOC(=O)c1c(-c2ccccc2F)nn2c1SCCCC2]

A solution of intermediate 24A (135 mg, 0.443 mmol), 2-fluorophenylboronic acid (124 mg, 0.886 mmol), XPhos (32 mg, 0.066 mmol) and K₃PO₄ (141 mg, 0.665 mmol) in 3:2 THF:water (10 mL) was sparged with N₂, Pd-170 (22 mg, 0.033 mmol) added, and the reaction heated at rt for 72 h. Analogous workup to that described for intermediate followed by purification by flash chromatography [0-100% (10% MeOH in ethyl acetate) in iso-hexanes] afforded a colourless oil (54 mg, 59%). LCMS (method B): m/z 321.3 [M+H]⁺ at 0.66 min.

### 43A Ethyl 4-(2-fluoro-4-methylsulfonylphenyl)-7-oxa-2,3-diazatricyclo[6.2.1.02,6]undeca-3,5-diene-5-carboxylate

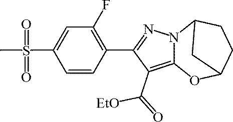 [SMILES: CCOC(=O)c1c(-c2ccc(S(C)(=O)=O)cc2F)nn2c1OC1CCC2C1]

A reaction vessel was charged with intermediate 23G (400 mg, 1.33 mmol), 2-fluoro-4-(methylsulfonyl)phenylboronic acid (434 mg, 1.99 mmol) and XPhos Pd G2 (78 mg, 0.10 mmol). The vessel was evacuated and refilled with N₂ (3×). THF (6 mL) and K₃PO₄ (2 M aq; 1.33 mL, 2.66 mmol), both been degassed with N₂, were added and the reaction heated at 80° C. overnight. The volatiles were removed under reduced pressure and the residue purified by flash chromatography (20-100% EtOAc in heptane) to afford a white solid (145 mg, 27%). ¹H NMR (400 MHz, DMSO-d₆) δ 7.85 - 7.75 (m, 2H), 7.75 - 7.65 (m, 1H), 5.32 - 5.20 (m, 1H), 4.86 - 4.74 (m, 1H), 4.02 (q, J = 7.1 Hz, 2H), 3.32 (s, 3H), 2.37 - 2.28 (m, 1H), 2.25 - 1.98 (m, 5H), 1.05 (t, J = 7.1 Hz, 3H). LRMS (APCI+) m/z 395.0 [M+H]⁺.

### 33A Ethyl 2-[6-(Cyclopropylamino)-2-Fluoropyridin-3-yl]-5,6, 7,8-Tetrahydropyrazolo[5,1-b][1,3]Oxazepine-3-Carboxylic Acid

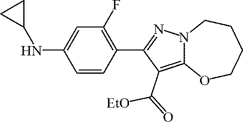 [SMILES: CCOC(=O)c1c(-c2ccc(NC3CC3)cc2F)nn2c1OCCCC2]

DIPEA (105 µL, 0.603 mmol) and cyclopropylamine (195 µL, 2.815 mmol) were added to a solution of intermediate 25B (162 mg, 0.500 mmol) in DMSO (1 mL) and the reaction heated at 50° C. for 5 h. The reaction was cooled to rt, partitioned between EtOAc (10 mL) and water (10 mL), and the separated aqueous layer extracted with EtOAc (3× 10 mL). The combined organics were washed with brine (20 mL), dried (Na₂SO₄) and concentrated under reduced pressure. Purification by flash chromatography [0-60% (10% MeOH in EtOAc) in iso-hexanes] afforded ethyl 2-[2-(cyclopropylamino)-6-fluoropyridin-3-yl]-5,6,7,8-tetrahydropyrazolo[5,1-b][1,3]oxazepine-3-carboxylate as a minor product [LCMS (method A): m/z 361.4 [M+H]⁺ at 1.37 min] followed by the major product intermediate 33A as a white solid (80 mg, 44%). LCMS (method A): m/z 361.4 [M+H]⁺ at 1.26 min.¹H NMR (500 MHz, DMSO-d₆) δ 7.64 - 7.54 (m, 1H), 7.27 (d, J = 2.5 Hz, 1H), 6.49 (dd, J = 8.2, 1.8 Hz, 1H), 4.28 - 4.13 (m, 4H), 4.07 (q, J = 7.1 Hz, 2H), 2.08 - 1.97 (m, 2H), 1.89 - 1.79 (m, 2H), 1.11 (t, J = 7.1 Hz, 3H), 0.73 (d, J = 6.8, 4.6 Hz, 2H), 0.48 - 0.41 (m, 2H).

### 34A Ethyl 2-[6-(ethylamino)-2-fluoropyridin-3-yl]-5-methyl-5,6,7,8-tetrahydropyrazolo[5,1-b][1,3]oxazepine-3-carboxylate

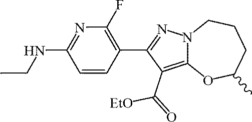 [SMILES: CCNc1ccc(-c2nn3c(c2C(=O)OCC)OC(C)CCC3)c(F)n1]

DIPEA (0.55 mL, 3.16 mmol) and ethylamine (2.0 M in THF; 4 mL, 5.09 mmol) were added to a solution of intermediate 25C (530 mg, 1.57 mmol) in DMSO (3 mL) and the reaction heated by MWI at 130° C. for 30 min. Analogous workup to that described for intermediate 33A followed by purification by flash chromatography (20-80% EtOAc in iso-hexanes) afforded a pale yellow solid (206 mg, 35%). LCMS (method A): m/z 363.4 [M+H]⁺ at 1.34 min. ^{l}H NMR (500 MHz, DMSO-d₆) 7.62 (dd, J = 9.5, 8.1 Hz, 1H), 6.23 (dd, J = 8.2, 1.9 Hz, 1H), 4.59 - 4.55 (m, 1H), 4.40 - 4.35 (m, 1H), 4.25 - 4.16 (m, 2H), 4.13 - 4.07 (m, 2H), 3.36 - 3.27 (m, 2H), 2.13 - 2.06 (m, 1H), 2.09 - 1.99 (m, 1H), 1.99 - 1.88 (m, 1H), 1.88 - 1.75 (m, 1H), 1.51 (d, J = 6.3 Hz, 3H), 1.24 (q, J = 7.3 Hz, 3H), 1.24 (q, J = 7.3 Hz, 3H).

### 34B Ethyl 2-[2-fluoro-6-(propan-2-ylamino)pyridin-3-yl]-5-methyl-5,6,7,8-tetrahydropyrazolo[5,1-b][1,3]oxazepine-3-carboxylate

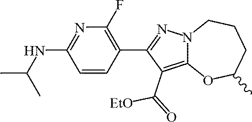 [SMILES: CCOC(=O)c1c(-c2ccc(NC(C)C)nc2F)nn2c1OC(C)CCC2]

Prepared by an analogous procedure to that described for intermediate 34A. LCMS (method A): m/z 377.5 [M+H]⁺ at 1.44 min. ¹H NMR (500 MHz, DMSO-d₆) 7.61 (dd, J = 9.5, 8.1 Hz, 1H), 6.21 (dd, J = 8.2, 1.9 Hz, 1H), 4.49 - 4.44 (m, 1H), 4.41 - 4.34 (m, 1H), 4.27 - 4.14 (m, 2H), 4.14 - 4.06 (m, 1H), 3.95 - 3.85 (m, 1H), 2.10 (d, J = 14.5 Hz, 1H), 2.03 - 1.99 (m, 1H), 1.99 - 1.88 (m, 1H), 1.86 - 1.75 (m, 1H), 1.51 (d, J = 6.3 Hz, 3H), 1.27 - 1.20 (m, 9H).

### 35A Ethyl 6,6-Difluoro-2-[6-(Propan-2-Ylamino)Pyridin-3-yl]-7,8-Dihydro-5H-Pyrazolo[5,1-b J[1,3 Joxazepine-3-Carboxylate

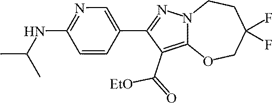 [SMILES: CCOC(=O)c1c(-c2ccc(NC(C)C)nc2)nn2c1OCC(F)(F)CC2]

DIPEA (597 µL, 3.43 mmol) was added to a solution of isopropylamine (147 µL, 1.71 mmol) and intermediate 29B (117 mg, 0.343 mmol) in DMSO (5 mL) and the reaction heated at 70° C. for 3 h. Analogous workup to that described for intermediate 33A followed by purification by flash chromatography (0-100% MTBE in heptane) afforded a colourless oil (44 mg, 34%). LCMS (method A): m/z 381.3 [M+H]⁺ at 0.79 min.

### 44A Ethyl 4-[6-(Propan-2-Ylamino)pyridin-3-yl]-7-Oxa-2,3-Diazatricyclo[6.2.1.02,6]Undeca-3,5-Diene-5-Carboxylate

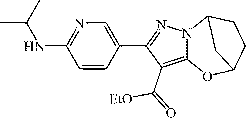 [SMILES: CCOC(=O)c1c(-c2ccc(NC(C)C)nc2)nn2c1OC1CCC2C1]

A solution of intermediate 42A (253 mg, 0.80 mmol), DIPEA (1.389 mL, 7.97 mmol) and isopropylamine (685 µL, 7.97 mmol) in DMSO (5 mL) was heated by MWI at 130° C. for 8 h. Further isopropylamine (343 µL, 3.99 mmol) was added and the reaction heated by MWI at 130° C. for 8 h. The reaction was diluted with water (15 mL) and extracted with EtOAc (2× 15 mL). The organic extracts were washed with brine (20 mL), passed through a phase separation cartridge and the solvent removed under reduced pressure to afford an orange solid (241 mg, 83%). LCMS (method A): m/z 357.4 [M+H]⁺ at 0.70 min.

### 36A Ethyl 2-(2-fluorophenyl)-4,4-dioxo-5,6,7,8-tetrahydropyrazolo[5,1-b][1,3]thiazepine-3-carboxylate

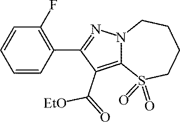 [SMILES: CCOC(=O)c1c(-c2ccccc2F)nn2c1S(=O)(=O)CCCC2]

meta-Chloroperoxybenzoic acid (81 mg, 0.328 mmol) was added to a cooled (0° C.) solution of intermediate 32A (42 mg, 0.131 mmol) in CH₂C1₂ (5 mL) and the reaction stirred at rt for 72 h. The reaction was diluted with CH₂C1₂ (25 mL) and quenched with 10% w/v aq. Na₂S₂O₃ (25 mL). The separated organic phase was washed with sat. aq. NaHCO₃ (3× 10 mL) and brine (10 mL), passed through a phase separation cartridge and concentrated under reduced pressure to afford a colourless oil (45 mg, 94%) which was used without further purification. LCMS (method B): m/z 353.4 [M+H]⁺ at 0.61 min.

### 37A 2-Phenyl-5,6,7,8-Tetrahydropyrazolo[5,1-b][1,3]Oxazepine-3-Carboxylic Acid

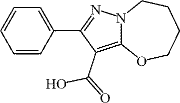 [SMILES: O=C(O)c1c(-c2ccccc2)nn2c1OCCCC2]

LiOH (1.5 M aq., 3 mL, 4.55 mmol) was added to a solution of intermediate 25A (130 mg, 0.46 mmol) in THF:MeOH (1:1, 6 mL) and the reaction stirred at 40° C. over the weekend. The reaction was cooled to rt, washed with MTBE (3× 10 mL), then acidified with 1 M aq. HC1 to pH ≈ 1, and extracted with CHC1₃:iPrOH (~3:1, 3× 10 mL). The combined organic extracts were dried (Na₂SO₄) and concentrated under reduced pressure to afford a white solid (120 mg, 97%) which was used without further purification. LCMS (method A): m/z 259.4 [M+H]⁺ at 0.94 min.¹H NMR (500 MHz, DMSO-d₆) δ 12.04 (s, 1H), 7.61 - 7.57 (m, 2H), 7.39 - 7.32 (m, 3H), 4.26 - 4.14 (m, 4H), 2.06 - 1.98 (m, 2H), 1.89 - 1.79 (m, 2H).

The following intermediates were prepared by an analogous procedure to intermediate 37A.

| | | | | |
|---|---|---|---|---|
| Preparatory Example | Name | Structure | ¹H NMR δ (500 MHz, DMSO) | LCMS (ES+) m/z |
| 37B | 2-(2-Fluorophenyl)-5,6,7,8-tetrahydropyrazo1o[5,1-b][1,3]oxazepine-3-carboxylic acid | 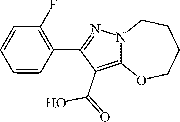 [SMILES: O=C(O)c1c(-c2ccccc2F)nn2c1OCCCC2] | 11.95 (s, 1H), 7.44 - 7.38 (m, 2H), 7.24 - 7.15 (m, 2H), 4.26 - 4.15 (m, 4H), 2.07 - 1.97 (m, 2H), 1.88 - 1.79 (m, 2H). | (method A) 277.4 [M+H]⁺ at 0.92 min |
| 37C | 2-(5-Methylpyridin-3-yl)-5,6,7,8-tetrahydropyrazolo[5,1-b][1,3]oxazepine-3-carboxylic acid | 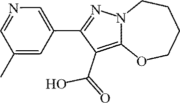 [SMILES: Cc1cncc(-c2nn3c(c2C(=O)O)OCCCC3)c1] | 12.14 (s, 1H), 8.57 - 8.53 (m, 1H), 8.39 - 8.35 (m, 1H), 7.79 - 7.75 (m, 1H), 4.27 - 4.21 (m, 2H), 4.22 - 4.17 (m, 2H), 2.34 -2.30 (m, 3H), 2.02 (d, J = 5.9 Hz, 2H), 1.84 (s, 2H). | (method A) 274.2 [M+H]⁺ at 0.49 min |

### 38A 2-Fluorophenyl)-8-Methyl-5,6,7,8-Tetrahydropyrazolo[5,1-b][1,3]Oxazepine-3-Carboxylic Acid

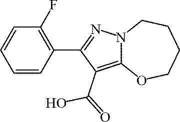 [SMILES: O=C(O)c1c(-c2ccccc2F)nn2c1OCCCC2]

LiOH (85 mg, 3.55 mmol) was added to a solution of intermediate 30A (140 mg, 0.44 mmol) in 1: 1: 1 MeOH:THF:water (3 mL) and the reaction heated at 50° C. for 2 h. The reaction was cooled to rt, acidified with 1 M aq. HC1 (5 mL), and extracted with CH₂C1₂ (3× 10 mL). The combined organics were passed through a phase separator containing brine (20 mL) and concentrated under pressure to afford a white solid (122 mg, 96%) which was used without further purification. LCMS (method A): m/z 291.3 [M+H]⁺ at 1.04 min.¹H NMR (500 MHz, DMSO-d₆) δ 11.94 (s, 1H), 7.46 - 7.37 (m, 2H), 7.24 -7.15 (m, 2H), 4.64 - 4.56 (m, 1H), 4.35 - 4.27 (m, 1H), 4.09 - 4.01 (m, 1H), 2.23 - 2.14 (m, 1H), 2.01 - 1.91 (m, 2H), 1.89 - 1.81 (m, 1H), 1.43 (d, J = 6.9 Hz, 3H).

The following intermediates were prepared by an analogous procedure to intermediate 38A.

| | | | | |
|---|---|---|---|---|
| Preparatory Example | Name | Structure | ¹H NMR δ (500 MHz, DMSO-d₆) | LCMS (ES+) m/z |
| 38B | 2-(2-Fluorophenyl)-5-methyl-5,6,7,8-tetrahydropyrazo1o[5,1-b][1,3]oxazepine-3-carboxylic acid | 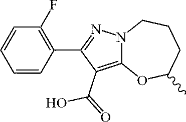 [SMILES: CC1CCCn2nc(-c3ccccc3F)c(C(=O)O)c2O1] | 11.90 (s, 1H), 7.45 - 7.37 (m, 2H), 7.24 - 7.15 (m, 2H), 4.35 -4.27 (m, 1H), 4.16 - 4.08 (m, 2H), 2.10 - 2.03 (m, 1H), 1.97 -1.93 (m, 1H), 1.93 - 1.79 (m, 1H), 1.78 - 1.66 (m, 1H), 1.38 (d, J = 6.3 Hz, 3H). | (method A) 291.3 [M+H]⁺ at 1.03 min |
| 38C | 2-(2,4-Difluorophenyl)-5-methyl-5,6,7,8-tetrahydropyrazo1o[5,1-b][1,3]oxazepine-3-carboxylic acid | 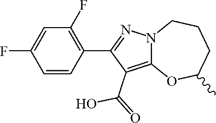 | 11.82 (s, 1H), 7.45 (dd, J = 10.0, 8.2 Hz, 1H), 6.87 (d, J = 7.6 Hz, 1H), 6.30 (dd, J = 8.2, 1.7 Hz, 1H), 4.30 - 4.22 (m, 1H), 4.13 -4.02 (m, 2H), 3.95 - 3.84 (m, 1H), 2.07 - 2.01 (m, 1H), 1.97 -1.90 (m, 1H), 1.88 - 1.77 (m, | (method A) 349.3 [M+H]⁺ at 1.12 min |
| | | | 1H), 1.74 - 1.63 (m, 1H), 1.36 (d, J = 6.3 Hz, 3H), 1.14 (d, J = 6.4 Hz, 6H). | |
| 38D | 2-[6-(Ethylamino)-2-fluoropyridin-3-yl]-5-methyl-5,6,7,8-tetrahydropyrazolo[5,1-b][1,3]oxazepine-3-carboxylic acid | 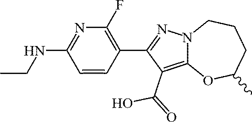 [SMILES: CCNc1ccc(-c2nn3c(c2C(=O)O)OC(C)CCC3)c(F)n1] | 11.81 (s, 1H), 7.47 (dd, J = 9.9, 8.2 Hz, 1H), 6.98 (s, 1H), 6.32 (dd, J = 8.2, 1.8 Hz, 1H), 4.30 -4.22 (m, 1H), 4.13 - 4.02 (m, 2H), 3.24 - 3.19 (m, 2H), 2.04 (d, J = 15.0 Hz, 1H), 1.93 (s, 1H), 1.82 (q, J = 12.4, 11.6 Hz, 1H), 1.69 (dt, J = 14.2, 11.3 Hz, 1H), 1.36 (d, J = 6.3 Hz, 3H), 1.13 (t, J = 7.2 Hz, 3H). | (method A) 335.3 [M+H]⁺ at 1.01 min. |
| 38E | 2-[2-Fluoro-6-(propan-2-ylamino)pyridin-3-yl]-5-methyl-5,6,7,8-tetrahydropyrazolo[5,1-b][1,3]oxazepine-3-carboxylic acid | 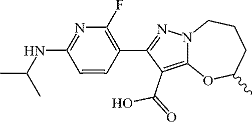 [SMILES: CC(C)Nc1ccc(-c2nn3c(c2C(=O)O)OC(C)CCC3)c(F)n1] | 11.82 (s, 1H), 7.45 (dd, J = 10.0, 8.2 Hz, 1H), 6.87 (d, J = 7.6 Hz, 1H), 6.30 (dd, J = 8.2, 1.7 Hz, 1H), 4.30 - 4.22 (m, 1H), 4.13 -4.02 (m, 2H), 3.95 - 3.84 (m, 1H), 2.07 - 2.01 (m, 1H), 1.97 -1.90 (m, 1H), 1.88 - 1.77 (m, 1H), 1.74 - 1.63 (m, 1H), 1.36 (d, J = 6.3 Hz, 3H), 1.14 (d, J = 6.4 Hz, 6H). | (method A) 349.3 [M+H]⁺ at 1.12 min |
| 38F | 2-(2-Fluorophenyl)-5,5-dimethyl-7,8-dihydro-6H-pyrazolo[5,1-b][1,3]oxazepine-3-carboxylic acid | 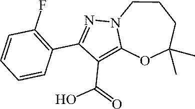 [SMILES: CC1(C)CCCn2nc(-c3ccccc3F)c(C(=O)O)c2O1] | 11.85 (s, 1H), 7.44 - 7.37 (m, 2H), 7.24 - 7.15 (m, 2H), 4.21 -4.15 (m, 2H), 2.01 - 1.95 (m, 2H), 1.87 - 1.80 (m, 2H), 1.32 (s, 6H). | (method A) 305.3 [M+H]⁺ at 1.09 min |
| 38G | 4-(2-Fluorophenyl)-7-oxa-2,3-diazatricyclo[6.2.1.0 ^{2,6}]undeca-3,5-diene- 5-carboxylic acid | 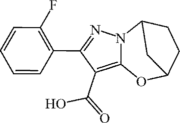 [SMILES: O=C(O)c1c(-c2ccccc2F)nn2c1OC1CCC2C1] | 11.77 (s, 1H), 7.47 - 7.35 (m, 2H), 7.26 - 7.14 (m, 2H), 5.23 -5.18 (m, 1H), 4.77 - 4.72 (m, 1H), 2.30 - 2.24 (m, 1H), 2.21 -2.11 (m, 2H), 2.11 - 2.06 (m, 1H), 2.06 - 1.97 (m, 2H). | (method A) 289.3 [M+H]⁺ at 0.87 min |

### 39A 2-(Cyclopropylamino)-2-Fluoropyridin-3-yl]-5,6,7,8-Tetrahydropyrazolo[5,1-b][1,3]Oxazepine-3-Carboxylic Acid

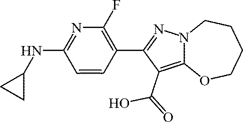 [SMILES: O=C(O)c1c(-c2ccc(NC3CC3)nc2F)nn2c1OCCCC2]

LiOH (1.5 M aq; 1.29 mL, 1.94 mmol) was added to a solution of intermediate 33A (70 mg, 0.194 mmol) in THF:MeOH (1:1, 2 mL) and the reaction heated at 40° C. overnight. The mixture was cooled to rt, 1 M aq. HC1 (1.3 mL) added and the mixture was concentrated under reduced pressure to afford an orange solid (64.5 mg, 100%) which was used without further purification. LCMS (method B): 333.8 [M+H]⁺ at 0.49 min.

### 45A 4-Fluoro-4-Methylsulfonylphenyl)-7-Oxa-2,3-Diazatricyclo[6.2.1.02,6]Undeca-3,5-Diene-5-Carboxylic Acid

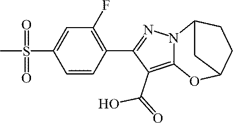 [SMILES: CS(=O)(=O)c1ccc(-c2nn3c(c2C(=O)O)OC2CCC3C2)c(F)c1]

A solution of intermediate 43A (140 mg, 0.355 mmol) and 5 M aq. NaOH (0.57 mL, 2.84 mmol) in EtOH (10 mL) was heated to 60° C. overnight. The volatiles were removed under reduced pressure, acidified with 1 M aq. HC1 to (pH ≈ 2) and extracted with CHC1₃:iPrOH (3: 1; 3 × 15 mL). The combined organics were washed with water and brine (15 mL) each, dried (MgSO₄) and the solvent removed under reduced pressure to afford an off-white solid (129 mg, 99%). ¹H NMR (400 MHz, DMSO-d₆) δ 11.98 (s, 1H), 7.83 - 7.73 (m, 2H), 7.72 - 7.63 (m, 1H), 5.28 - 5.19 (m, 1H), 4.84 - 4.73 (m, 1H), 3.33 (s, 3H), 2.36 - 2.26 (m, 1H), 2.22 - 1.88 (m, 5H). LRMS (APCI+) m/z 367.2 [M+H]⁺.

### EXAMPLES

### 1. N-[(3S)-2-Oxo-5-Phenyl-1,3-Dihydro-1,4-Benzodiazepin-3-yl]-2-Phenyl-5,6,7,8-Tetrahydropyrazolo[5,1-b] [1,3]Oxazepine-3-Carboxamide (Procedure A)

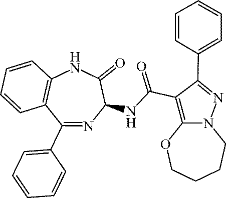 [SMILES: O=C(N[C@H]1N=C(c2ccccc2)c2ccccc2NC1=O)c1c(-c2ccccc2)nn2c1OCCCC2]

NEt₃ (64 µL, 0.459 mmol), HATU (84 mg, 0.221 mmol) and (3S)-3-amino-5-phenyl-1,3-dihydro-1,4-benzodiazepin-2-one (55 mg, 0.220 mmol) were added to a solution of intermediate 37A (57 mg, 0.220 mmol) in DMF (2 mL) and the reaction stirred at 40° C. for 1 h. The reaction was diluted with EtOAc (40 mL), washed with brine (4×20 mL). The layers were separated and the aqueous layer was extracted with CHC1₃:iPrOH (3:1; 30 mL). The organic layers were combined, concentrated under reduced pressure, and purified by flash chromatography (0-5% MeOH in CH₂Cl₂) to afford a white solid (69 mg, 64%). LCMS (method C): m/z 492.4 [M+H]⁺ at 4.27 min. ¹H NMR (500 MHz, DMSO-d₆) δ 10.99 (s, 1H), 8.59 (d, J = 7.5 Hz, 1H), 7.71 - 7.68 (m, 2H), 7.67 - 7.62 (m, 1H), 7.55 - 7.50 (m, 1H), 7.50 - 7.43 (m, 4H), 7.36 - 7.29 (m, 5H), 7.29 - 7.25 (m, 1H), 5.33 (d, J = 7.5 Hz, 1H), 4.48 - 4.42 (m, 1H), 4.42 - 4.36 (m, 1H), 4.33 - 4.28 (m, 2H), 2.18 - 2.08 (m, 2H), 1.95 - 1.86 (m, 2H).

The following compounds of the invention were prepared with (3S)-3-amino-5-phenyl-1,3-dihydro-1,4-benzodiazepin-2-one or (3S)-3-amino-9-fluoro-5-phenyl-1,3-dihydro-1,4-benzodiazepin-2-one by an analogous procedure to that described for the compound of Example 1.

### Table 2 - Example Compounds Prepared by Amide Coupling Procedure A

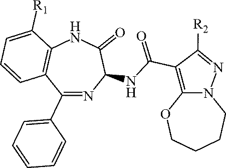

| Example | Name | R¹ | R² | ¹H NMR δ (500 MHz, DMSO-d₆) δ | LCMS (ES+) m/z |
|---|---|---|---|---|---|
| 2 | N-[(3S)-9-Fluoro-2-oxo-5-phenyl-1,3-dihydro-1,4-benzodiazepin-3-yl]-2-(2-fluorophenyl)-5,6,7,8-tetrahydropyrazolo[5,1-b][1,3]oxazepine-3-carboxamide | F | 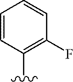 | 10.96 (s, 1H), 8.35 (d, J = 7.6 Hz, 1H), 7.60 - 7.54 (m, 1H), 7.54 -7.50 (m, 1H), 7.50 - 7.43 (m, 4H), 7.41 - 7.35 (m, 2H), 7.32 - 7.25 (m, 1H), 7.20 - 7.09 (m, 3H), 5.34 (d, J = 7.5 Hz, 1H), 4.50 - 4.44 (m, 1H), 4.44 - 4.37 (m, 1H), 4.33 -4.28 (m, 2H), 2.19 - 2.08 (m, 2H), 1.96 - 1.82 (m, 2H). | (method C) 528.4 [M+H]⁺ at 4.16 min |
| 3 | 2-(5-Methylpyridin-3-yl)-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide | H | 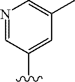 | 11.00 (s, 1H), 8.62 (d, J = 2.0 Hz, 1H), 8.60 (d, J = 7.4 Hz, 1H), 8.34 (d, J = 2.1 Hz, 1H), 7.86 (s, 1H), 7.64 (t, J = 7.8 Hz, 1H), 7.51 (t, J = 7.0 Hz, 1H), 7.50 - 7.40 (m, 4H), 7.32 (d, J = 8.0 Hz, 2H), 7.26 (t, J = 7.5 Hz, 1H), 5.33 (d, J = 7.4 Hz, 1H), 4.49 - 4.35 (m, 2H), 4.32 (t, J = 5.5 Hz, 2H), 2.30 (s, 3H), 2.16 -2.10 (m, 2H), 1.94 - 1.88 (m, 2H). | (method C) 507.4 [M+H]⁺ at 2.40 min |
| 4 | N-[(3S)-9-Fluoro-2-oxo-5-phenyl-1,3-dihydro-1,4-benzodiazepin-3-yl]-2-(5-methylpyridin-3-yl)-5,6,7,8-tetrahydropyrazolo[5,1-b][1,3]oxazepine-3-carboxamide | F | 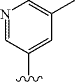 | 10.97 (s, 1H), 8.63 - 8.59 (m, 2H), 8.35 (s, 1H), 7.86 (s, 1H), 7.62 -7.56 (m, 1H), 7.55 - 7.48 (m, 3H), 7.47 - 7.43 (m, 2H), 7.32 (ddd, J = 8.1, 4.9 Hz, 1H), 7.16 (d, J = 7.9 Hz, 1H), 5.41 (d, J = 7.3 Hz, 1H), 4.49 - 4.37 (m, 2H), 4.34 - 4.30 (m, 2H), 2.30 (s, 3H), 2.15 - 2.10 (m, 2H), 1.95 - 1.87 (m, 2H) | (method C) 525.4 [M+H]⁺ at 2.39 min |

### 5. 2-Fluorophenyl)-5,5-Dimethyl-N-[(3S)-2-Oxo-5-Phenyl-1,3-Dihydro-1,4-Benzodiazepin-3-yl]-7,8-Dihydro-6H-Pyrazolo [5,1-b] [1,3]Oxazepine-3-Carboxamide (Procedure B)

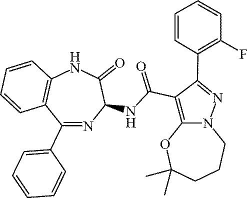 [SMILES: CC1(C)CCCn2nc(-c3ccccc3F)c(C(=O)N[C@H]3N=C(c4ccccc4)c4ccccc4NC3=O)c2O1]

NEt₃ (30 µL, 0.215 mmol), HATU (38 mg, 0.100 mmol) and (3S)-3-amino-5-phenyl-1,3-dihydro-1,4-benzodiazepin-2-one (25 mg, 0.100 mmol) were added to a solution of intermediate 38F (30 mg, 0.100 mmol) in DMF (0.5 mL) and the reaction stirred at rt for 2 h. The reaction was quenched with water (20 mL), and the resultant precipitate filtered and washed with water. The precipitate was dissolved with CH₂Cl₂ (10 mL), passed through a phase separator containing brine (10 mL) and concentrated under reduced pressure. Purification by flash chromatography (10-60% MeOH in CH₂Cl₂) afforded white solid (41 mg, 77%). LCMS (method C): m/z 538.3 [M+H]⁺ at 4.65 min.¹H NMR (500 MHz, DMSO-d₆) δ 10.97 (s, 1H), 8.31 (d, J = 8.0 Hz, 1H), 7.67 -7.60 (m, 1H), 7.54 - 7.47 (m, 1H), 7.47 - 7.40 (m, 4H), 7.40 - 7.33 (m, 2H), 7.33 - 7.28 (m, 2H), 7.28 - 7.22 (m, 1H), 7.19 - 7.14 (m, 1H), 7.14 - 7.09 (m, 1H), 5.30 (d, J = 8.0 Hz, 1H), 4.34 - 4.20 (m, 2H), 2.14 - 2.08 (m, 2H), 1.93 - 1.89 (m, 2H), 1.58 (s, 3H), 1.49 (s, 3H).

The following compounds of the invention were prepared by an analogous procedure to that described for the compound of Example 5.

### Table 3 - Example Compounds Prepared by Amide Coupling Procedure B

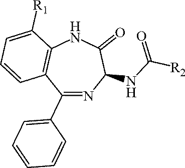

| | | | | | |
|---|---|---|---|---|---|
| Example | Name | R¹ | R² | ¹H NMR δ (500 MHz, DMSO-d₆) δ | LCMS (ES+) m/z |
| 6 | N-[(3S)-9-Fluoro-2-oxo-5-phenyl-1,3-dihydro-1,4-benzodiazepin-3-yl]-2-(2-fluorophenyl)-5,5-dimethyl-7,8-dihydro-6H-pyrazolo[5,1-b][1,3]oxazepine-3-carboxamide | F | 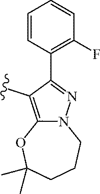 | 10.95 (s, 1H), 8.33 (d, J = 7.9 Hz, 1H), 7.61 - 7.55 (m, 1H), 7.55 - 7.49 (m, 1H), 7.51 - 7.41 (m, 4H), 7.41 - 7.34 (m, 2H), 7.37 - 7.26 (m, 1H), 7.20 - 7.13 (m, 1H), 7.16 - 7.08 (m, 2H), 5.39 (d, J = 7.9 Hz, 1H), 4.34 -4.20 (m, 2H), 2.14 - 2.07 (m, 2H), 1.93 - 1.89 (m, 2H), 1.59 (s, 3H), 1.49 (s, 3H). | (method C) 556.3 [M+H]⁺ at 4.73 min |
| 7 | 2-(2-Fluorophenyl)-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide | H | 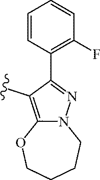 | 10.99 (s, 1H), 8.33 (d, J = 7.6 Hz, 1H), 7.66 - 7.59 (m, 1H), 7.54 - 7.46 (m, 1H), 7.48 - 7.40 (m, 4H), 7.42 - 7.34 (m, 2H), 7.33 - 7.27 (m, 2H), 7.28 - 7.21 (m, 1H), 7.20 - 7.10 (m, 2H), 5.26 (d, J = 7.6 Hz, 1H), 4.51 -4.37 (m, 2H), 4.34 - 4.28 (m, 2H), 2.17 - 2.10 (m, 2H), 1.96 -1.88 (m, 2H). | (method C) 510.5 [M+H]⁺ at 4.09 min. |
| 8 | N-[(3S)-9-Fluoro-2-oxo-5-phenyl-1,3-dihydro-1,4-benzodiazepin-3-yl]-2-phenyl-5,6,7,8-tetrahydropyrazolo[5,1-b][1,3]oxazepine-3-carboxamide | F | 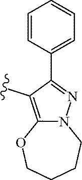 | 10.96 (s, 1H), 8.61 (d, J = 7.4 Hz, 1H), 7.72 - 7.66 (m, 2H), 7.62 - 7.57 (m, 1H), 7.56 - 7.49 (m, 3H), 7.49 - 7.44 (m, 2H), 7.37 - 7.28 (m, 4H), 7.17 (d, J = 7.9 Hz, 1H), 5.41 (d, J = 7.4 Hz, 1H), 4.49 - 4.42 (m, 1H), 4.41 -4.34 (m, 1H), 4.33 - 4.27 (m, 2H), 2.16 - 2.10 (m, 2H), 1.96 -1.86 (m, 2H). | (method C) 510.4 [M+H]⁺ at 4.34 min |
| 9 | 2-[6-(Cyclopropylamino)-2-fluoropyridin-3-yl]-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide | H | 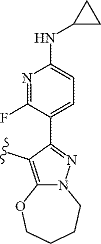 | 10.99 (s, 1H), 8.33 (d, J = 7.6 Hz, 1H), 7.66 - 7.61 (m, 1H), 7.57 - 7.49 (m, 2H), 7.48 - 7.41 (m, 4H), 7.33 - 7.29 (m, 2H), 7.29 - 7.24 (m, 1H), 7.19 (d, J = 2.5 Hz, 1H), 6.43 (dd, J = 8.2, 1.8 Hz, 1H), 5.28 (d, J = 7.6 Hz, 1H), 4.48 - 4.42 (m, 1H), 4.41 -4.35 (m, 1H), 4.31 - 4.26 (m, 2H), 2.17 - 2.08 (m, 2H), 1.93 - | (method C) 566.3 [M+H]⁺ at 4.08 min |
| | | | | 1.86 (m, 2H), 0.73 - 0.66 (m, 2H), 0.43 - 0.37 (m, 2H) | |

The following compounds of the invention were prepared by an analogous procedure to that described for the compound of Example 5 as 1:1 mixture of diastereomers. 0.5H corresponds to 1H of a diastereomeric peak in the ¹H NMR assignment. Examples 16 and 17 were prepared using 5 eq. of NEt₃ in the amide coupling step.

### Table 4 - Diastereomeric Example Compounds Prepared by Amide Coupling Procedure B

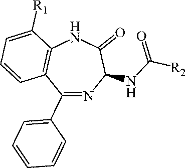

| Example | Name | R¹ | R² | ¹H NMR δ (500 MHz, DMSO-d₆) δ | LCMS (ES+) m/z |
|---|---|---|---|---|---|
| 10 | 2-[6-(Ethylamino)-2-fluoropyridin-3 -yl]-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide | H | 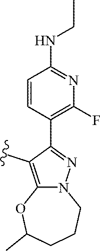 | 10.97 (s, 1H), 8.35 (d, J = 7.6 Hz, 0.5H), 8.28 (d, J = 8.0 Hz, 0.5H), 7.67 - 7.60 (m, 1H), 7.55 - 7.48 (m, 1H), 7.47 - 7.39 (m, 5H), 7.34 - 7.27 (m, 2H), 7.29 - 7.22 (m, 1H), 6.93 (s, 1H), 6.30 - 6.24 (m, 1H), 5.32 (d, J = 7.9 Hz, 1H), 5.28 (d, J = 7.5 Hz, 0H), 4.44 - 4.32 (m, 2H), 4.18 - 4.08 (m, 1H), 3.23 - 3.14 (m, 2H), 2.17 - 2.11 (m, 1H), 2.06 - 1.88 (m, 2H), 1.85 -1.74 (m, 1H), 1.64 (d, J = 6.3 Hz, 3H), 1.57 (d, J = 6.3 Hz, 0H), 1.13 -1.07 (m, 3H). | (method C) 568.4 [M+H]⁺ at 4.23 and 4.35 min |
| 11 | 2-[6-(Ethylamino)-2-fluoropyridin-3 -yl]-N-[(3S)-9-fluoro-2-oxo-5-phenyl-1,3-dihydro-1,4-benzodiazepin-3-yl]-5-methyl-5,6,7,8-tetrahydropyrazol o[5,1-b][1,3]oxazepine-3-carboxamide | F | 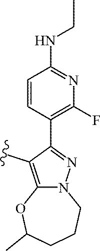 | 10.94 (s, 1H), 8.37 (d, J = 7.5 Hz, 0.5H), 8.30 (d, J = 7.9 Hz, 0.5H), 7.62 - 7.55 (m, 1H), 7.55 - 7.50 (m, 1H), 7.50 - 7.40 (m, 5H), 7.35 - 7.27 (m, 1H), 7.17 - 7.11 (m, 1H), 6.95 -6.90 (m, 1H), 6.30 - 6.24 (m, 1H), 5.40 (d, J = 7.9 Hz, 0.5H), 5.36 (d, J = 7.5 Hz, 0.5H), 4.46 - 4.32 (m, 2H), 4.18 - 4.08 (m, 1H), 3.26 - 3.14 (m, 2H), 2.17 - 2.11 (m, 1H), 2.04 - 1.92 (m, 2H), 1.85 - 1.74 (m, 1H), 1.65 (d, J = 6.3 Hz, 1.5H), 1.58 (d, J = 6.3 Hz, 1.5H), 1.13 - 1.07 (m, 3H) | (method C) 586.4 [M+H]⁺ at 4.31 and 4.43 min |
| 12 | 2-[2-Fluoro-6-(propan-2-ylamino)pyridin-3-yl]-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide | H | 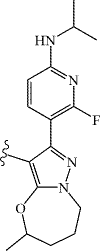 | 10.97 (s, 1H), 8.35 (d, J = 7.6 Hz, 0.5H), 8.29 (d, J = 7.9 Hz, 0.5H), 7.67 - 7.60 (m, 1H), 7.54 - 7.48 (m, 1H), 7.51 - 7.37 (m, 5H), 7.34 - 7.28 (m, 2H), 7.28 - 7.23 (m, 1H), 6.85 -6.79 (m, 1H), 6.29 - 6.23 (m, 1H), 5.32 (d, J = 8.0 Hz, 0.5H), 5.28 (d, J = 7.6 Hz, 0.5H), 4.44 - 4.32 (m, 2H), 4.18 - 4.08 (m, 1H), 3.92 - 3.82 (m, 1H), 2.17 - 2.11 (m, 1H), 2.05 - 1.90 (m, 2H), 1.85 - 1.74 (m, 1H), 1.64 (d, J = 6.2 Hz, 1.5H), 1.57 (d, J = 6.3 Hz, 1.5H), 1.15 - 1.09 (m, 6H). | (method A) 582.4 [M+H]⁺ at 1.51 and 1.54 min. |
| 13 | N-[(3S)-9-fluoro-2-oxo-5-phenyl-1,3-dihydro-1,4-benzodiazepin-3-yl]-2-[2-fluoro-6-(propan-2-ylamino)pyridin-3-yl]-5-methyl-5,6,7,8-tetrahydropyrazolo[5,1-b][1,3]oxazepine-3-carboxamide | F | 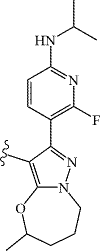 | 10.94 (s, 1H), 8.38 (d, J = 7.5 Hz, 0.5H), 8.31 (d, J = 7.9 Hz, 0.5H), 7.61 - 7.56 (m, 1H), 7.55 - 7.50 (m, 1H), 7.50 - 7.44 (m, 4H), 7.44 - 7.39 (m, 1H), 7.34 - 7.28 (m, 1H), 7.17 -7.11 (m, 1H), 6.85 - 6.79 (m, 1H), 6.29 - 6.23 (m, 1H), 5.40 (d, J = 7.9 Hz, 0.5H), 5.36 (d, J = 7.5 Hz, 0.5H), 4.44 - 4.31 (m, 2H), 4.18 - 4.08 (m, 1H), 3.92 - 3.82 (m, 1H), 2.17 - 2.11 (m, 1H), 2.04 - 1.91 (m, 2H), 1.85 -1.74 (m, 1H), 1.65 (d, J = 6.3 Hz, 1.5H), 1.58 (d, J = 6.3 Hz, 1.5H), 1.18 - 1.04 (m, 6H). | (method A) 600.4 [M+H]⁺ at 1.53 and 1.55 min. |
| 14 | 2-(2-Fluorophenyl)-8-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide | H | 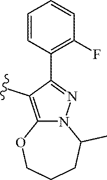 | 11.00 (s, 1H), 8.37 (d, J = 7.7 Hz, 0.5H), 8.33 (d, J = 7.6 Hz, 0.5H), 7.66 - 7.59 (m, 1H), 7.54 - 7.48 (m, 1H), 7.51 - 7.38 (m, 4H), 7.41 - 7.34 (m, 2H), 7.33 - 7.26 (m, 2H), 7.28 -7.22 (m, 1H), 7.20 - 7.09 (m, 2H), 5.29 - 5.23 (m, 1H), 4.71 - 4.67 (m, 1H), 4.62 - 4.56 (m, 0.5H), 4.55 -4.50 (m, 0.5H), 4.37 - 4.30 (m, 0.5H), 4.30 - 4.23 (m, 0.5H), 2.34 -2.30 (m, 1H), 2.09 - 2.00 (m, 2H), 1.94 - 1.91 (m, 1H), 1.48 (d, J = 6.9 Hz, 3H). | (method C) 524.4 [M+H]⁺ at 4.36 and 4.46 min |
| 15 | N-[(3S)-9-fluoro-2-oxo-5-phenyl-1,3-dihydro-1,4-benzodiazepin-3-yl]-2-(2-fluorophenyl)-8-methyl-5,6,7,8-tetrahydropyrazolo[5,1-b][1,3]oxazepine-3-carboxamide | F | 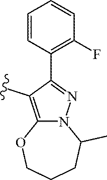 | 10.96 (s, 1H), 8.38 (d, J = 7.6 Hz, 0.5H), 8.33 (d, J = 7.5 Hz, 0.5H), 7.61 - 7.55 (m, 1H), 7.55 - 7.50 (m, 1H), 7.50 - 7.42 (m, 4H), 7.41 - 7.36 (m, 2H), 7.34 - 7.26 (m, 1H), 7.21 -7.10 (m, 3H), 5.35 (d, J = 7.5 Hz, 1H), 4.72 - 4.66 (m, 1H), 4.62 - 4.56 (m, 0.5H), 4.54 - 4.50 (m, 0.5H), 4.38 - 4.30 (m, 0.5H), 4.30 - 4.22 (m, 0.5H), 2.34 - 2.31 (m, 1H), 2.07 - 2.03 (m, 2H), 1.94 - 1.91 (m, 1H), 1.51 - 1.45 (m, 3H). | (method C) 542.4 [M+H]⁺ at 4.45 and 4.53 min |
| 16 | 2-(2,4-Difluorophenyl)-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide | H | 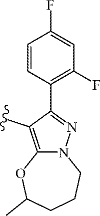 | 10.97 (d, J = 5.8 Hz, 1H), 8.37 (d, J = 7.5 Hz, 0.5H), 8.31 (d, J = 8.0 Hz, 0.5H), 7.63 (s, 1H), 7.54 - 7.47 (m, 1H), 7.47 - 7.37 (m, 5H), 7.33 - 7.28 (m, 2H), 7.27 - 7.22 (m, 1H), 7.21 -7.14 (m, 1H), 7.09 - 7.01 (m, 1H), 5.31 (d, J = 8.0 Hz, 0.5H), 5.27 (d, J = 7.5 Hz, 0.5H), 4.54 - 4.36 (m, 2H), 4.22 - 4.13 (m, 1H), 2.14 (s, 1H), 2.07 - 1.91 (m, 2H), 1.88 - 1.77 (m, 1H), 1.66 (d, J = 6.3 Hz, 1.5H), 1.58 (d, J = 6.3 Hz, 1.5H). | (method C) 560.4 [M+H]⁺ at 4.60 and 4.74 min |
| 17 | 2-(2,4-Difluorophenyl)-N-[(3S)-9-fluoro-2-oxo-5-phenyl-1,3-dihydro-1,4-benzodiazepin-3-yl]-5-methyl-5,6,7,8-tetrahydropyrazolo[5,1-b][1,3]oxazepine-3-carboxamide | F | 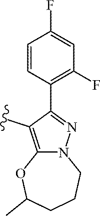 | 10.95 (s, 1H), 8.40 (d, J = 7.4 Hz, 0.5H), 8.33 (d, J = 7.9 Hz, 0.5H), 7.61 - 7.50 (m, 2H), 7.50 - 7.38 (m, 5H), 7.34 - 7.26 (m, 1H), 7.22 - 7.10 (m, 2H), 7.09 - 7.01 (m, 1H), 5.39 (d, J = 7.8 Hz, 0.5H), 5.35 (d, J = 7.4 Hz, 0.5H), 4.49 - 4.36 (m, 2H), 4.18 (td, J = 12.6, 7.1 Hz, 1H), 2.19 - 2.13 (m, 1H), 2.07 - 1.94 (m, 2H), 1.88 -1.77 (m, 1H), 1.66 (d, J = 6.3 Hz, 1.5H), 1.59 (d, J = 6.3 Hz, 1.5H). | Not available |
| 18 | 2-(2-Fluorophenyl)-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide | H | 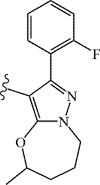 | 10.97 (s, 0.5H), 10.96 (s, 0.5H), 8.37 (d, J = 7.6 Hz, 0.5H), 8.30 (d, J = 8.0 Hz, 0.5H), 7.67 - 7.59 (m, 1H), 7.54 - 7.47 (m, 1H), 7.47 - 7.41 (m, 4H), 7.41 - 7.34 (m, 2H), 7.33 - 7.21 (m, 3H), 7.20 - 7.09 (m, 2H), 5.31 (d, J = 7.9 Hz, 0.5H), 5.27 (d, J = 7.6 Hz, 0.5H), 4.48 - 4.36 (m, 2H), 4.23 -4.13 (m, 1H), 2.19 - 2.13 (m, 1H), 2.08 - 1.94 (m, 2H), 1.89 - 1.77 (m, 1H), 1.66 (d, J = 6.3 Hz, 0.5H), 1.59 (d, J = 6.3 Hz, 1.5H). | (method C) 524.3 [M+H]⁺ at 4.38 min and 4.51 min. |
| 19 | N-((S)-9-fluoro-2-oxo-5-phenyl-2,3-dihydro-1H-benzo[b]azepin-3-yl)-2-(2-fluorophenyl)-5,6,7,8-tetrahydro-5,8-methanopyrazolo[ 5,1-b][1,3]oxazepine-3-carboxamide | F | 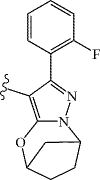 | 10.93 (s, 1H), 7.79 (d, J = 3.2 Hz, 0.5H), 7.77 (d, J = 3.2 Hz, 0.5H), 7.60 - 7.53 (m, 1H), 7.55 - 7.50 (m, 1H), 7.50 - 7.41 (m, 4H), 7.41 - 7.33 (m, 2H), 7.33 - 7.25 (m, 1H), 7.19 -7.12 (m, 1H), 7.16 - 7.08 (m, 2H), 5.47 - 5.43 (m, 1H), 5.34 (d, J = 7.6 Hz, 1H), 4.85 - 4.81 (m, 1H), 2.46 -2.40 (m, 1H), 2.27 - 2.24 (m, 1H), 2.24 - 2.13 (m, 3H), 2.08 - 2.05 (m, 1H) | (method C) 540.3 [M+H]⁺ at 4.07 min. |

### 20. N-[(3S)-9-Fluoro-2-Oxo-5-Phenyl-2,3-Dihydro-1H-1,4-Benzodiazepin-3-yl]-2-(2-Fluorophenyl)-SH,6H,7H,8H-Pyrazolo[3,2-b] [1,3]Thiazepine-3-Carboxamide (Procedure C)

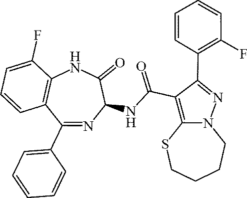 [SMILES: O=C(N[C@H]1N=C(c2ccccc2)c2cccc(F)c2NC1=O)c1c(-c2ccccc2F)nn2c1SCCCC2]

A solution of intermediate 32A (42 mg, 0.131 mmol) and LiOH (63 mg, 2.622 mmol) in MeOH:THF;water (1:1:1; 12 mL) was heated at 60° C. for 72 h. The reaction was cooled to rt, acidified with 1 M aq. HCl (10 mL) and the solvent removed under reduced pressure. The crude residue was dissolved in DMF (5 mL), NEt₃ (91 µL, 0.655 mmol) and (3S)-3-amino-9-fluoro-5-phenyl-1,3-dihydro-1,4-benzodiazepin-2-one (35 mg, 0.131 mmol) added, followed by HATU (50 mg, 0.131 mmol) and the reaction stirred at rt for 1 h. Analogous workup to that described for the compound of Example 5 followed by purification by flash chromatography [0-100% (10% MeOH in ethyl acetate) in iso-hexanes] afforded a white solid (40 mg, 56%). LCMS (method C): m/z 544.3 [M+H]⁺ at 4.40 min.¹H NMR (500 MHz, DMSO-d₆) δ 10.89 (s, 1H), 8.83 (d, J = 7.6 Hz, 1H), 7.62 - 7.51 (m, 2H), 7.54 - 7.42 (m, 5H), 7.44 - 7.38 7.41 (m, 1H), 7.30 (td, J = 8.1, 4.9 Hz, 1H), 7.25 - 7.20 (m, 1H), 7.22 - 7.13 (m, 2H), 5.38 (d, J = 7.6 Hz, 1H), 4.52 (q, J = 4.1, 2.6 Hz, 2H), 3.05 - 2.95 (m, 2H), 2.14 (s, 2H), 1.82 (s, 2H).

The following compounds of the invention were prepared by an analogous procedure to that described for the compound of Example 20.

### Table 5 - Example Compounds Prepared by Amide Coupling Procedure C

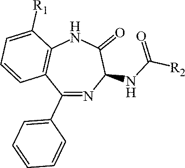

| Example | Name | R¹ | R² | ¹H NMR δ (500 MHz, DMSO-d₆) | LCMS (ES+) m/z |
|---|---|---|---|---|---|
| 21 | N-[(3S)-9-fluoro-2-oxo-5-phenyl-2,3-dihydro-1H-1,4-benzodiazepin-3-yl]-2-(2-fluorophenyl)-4,4-dioxo-5H,6H,7H,8H-4λ⁶- pyrazolo[3,2-b][1,3]thiazepine-3-carboxamide | F | 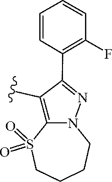 | 10.87 (s, 1H), 9.67 (d, J = 7.7 Hz, 1H), 7.79 (td, J = 7.7, 1.8 Hz, 1H), 7.61 (t, J = 9.2 Hz, 1H), 7.58 - 7.46 (m, 6H), 7.32 (tt, J = 8.1, 4.3 Hz, 2H), 7.27 (td, J = 7.5, 1.2 Hz, 1H), 7.19 (d, J = 7.9 Hz, 1H), 5.35 (d, J = 7.6 Hz, 1H), 4.61 (t, J = 5.2 Hz, 2H), 3.86 - 3.69 (m, 2H), 2.22 -2.15 (m, 2H), 2.02 - 1.90 (m, 2H). | (method C) 576.3 [M+H]⁺ at 3.82 min |
| 22 | 2-(6-Ethylpyridin-3-yl)-6,6-difluoro-N-[(3S)-9-fluoro-2-oxo-5-phenyl-2,3-dihydro-1H-1,4-benzodiazepin-3-yl]-5H,6H,7H,8H-pyrazolo[3,2-b][1,3]oxazepine-3-carboxamide | F | 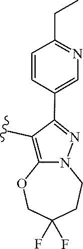 | 10.98 (s, 1H), 8.75 (dd, J = 2.3, 0.8 Hz, 1H), 8.67 (d, J = 7.4 Hz, 1H), 7.98 (dd, J = 8.1, 2.3 Hz, 1H), 7.60 (t, J = 9.2 Hz, 1H), 7.58 - 7.52 (m, 3H), 7.51 - 7.44 (m, 2H), 7.36 - 7.30 (m, 1H), 7.26 (d, J = 8.2, 0.8 Hz, 1H), 7.17 (d, J = 7.9 Hz, 1H), 5.41 (d, J = 7.4 Hz, 1H), 4.69 (t, J = 11.2 Hz, 2H), 4.43 - 4.37 (m, 2H), 2.77 (q, J = 7.6 Hz, 2H), 2.57 (s, 2H), 1.24 (t, J = 7.6 Hz, 3H). | (method C) 575.4 [M+H]⁺ at 2.73 min |
| 23 | 6,6-Difluoro-N-[(3S)-9-fluoro-2-oxo-5-phenyl-2,3-dihydro-1H-1,4-benzodiazepin-3-yl]-2-{6-[(propan-2-yl)amino]pyridin-3-yl}-5H,6H,7H,8H-pyrazolo[3,2-b][1,3]oxazepine-3-carboxamide | F | 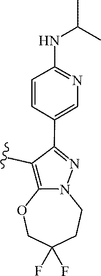 | 10.97 (s, 1H), 8.56 (d, J = 7.5 Hz, 1H), 8.29 (d, J = 2.4 Hz, 1H), 7.66 (dd, J = 8.7, 2.4 Hz, 1H), 7.60 (t, J = 9.2 Hz, 1H), 7.54 (d, J = 7.3 Hz, 3H), 7.51 -7.44 (m, 2H), 7.36 - 7.29 (m, 1H), 7.18 (d, J = 7.9 Hz, 1H), 6.48 (d, J = 7.7 Hz, 1H), 6.40 (d, J = 8.8 Hz, 1H), 5.41 (d, J = 7.4 Hz, 1H), 4.64 (t, J = 11.0 Hz, 2H), 4.37 - 4.31 (m, 2H), 4.07 - 3.97 (m, 1H), 2.51 (s, 2H), 1.14 (d, J = 6.4 Hz, 6H). | (method C) 604.5 [M+H]⁺ at 2.71 min |

The following compounds of the invention were prepared by an analogous procedure to that described for the compound of Example 20 as a 1:1 mixture of diastereomers. 0.5H corresponds to 1H of a diastereomeric peak in the ¹H NMR assignment

### Table 6 - Diastereomeric Example Compounds Prepared by Amide Coupling Procedure C

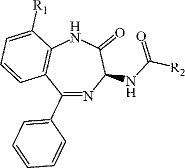

| Example | Name | R¹ | R² | ¹H NMR δ (500 MHz, DMSO-d₆) δ | LCMS (ES+) m/z |
|---|---|---|---|---|---|
| 24 | 5-Ethyl-2-(2-fluorophenyl)-N-[(3S)-2-oxo-5-phenyl-1,3-dihydro-1,4-benzodiazepin-3-yl]-5,6,7,8-tetrahydropyrazol o[5,1-b][1,3]oxazepine-3-carboxamide | H | 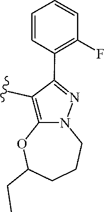 | 10.97 (s, 1H), 8.28 (d, J = 7.7 Hz, 0.5H), 8.24 (d, J = 7.9 Hz, 0.5H), 7.66 - 7.59 (m, 1H), 7.54 - 7.48 (m, 1H), 7.48 - 7.41 (m, 4H), 7.41 - 7.33 (m, 2H), 7.33 - 7.27 (m, 2H), 7.27 - 7.21 (m, 1H), 7.20 -7.09 (m, 2H), 5.29 (d, J = 2.1 Hz, 0.5H), 5.27 (d, J = 2.0 Hz, 0.5H), 4.46 - 4.38 (m, 1H), 4.31 - 4.23 (m, 1H), 4.22 - 4.13 (m, 1H), 2.17 (d, J = 13.7 Hz, 1H), 2.11 - 2.04 (m, 1H), 2.04 - 1.81 (m, 4H), 1.10 (t, J = 7.4 Hz, 1.5H), 1.05 (t, J = 7.4 Hz, 1.5H). | (method C) 538.3 [M+H]⁺ at 4.67 and 4.75 min |
| 25 | 5-Ethyl-N-[(3S)-9-fluoro-2-oxo-5-phenyl-1,3-dihydro-1,4-benzodiazepin-3-yl]-2-(2-fluorophenyl)-5,6,7,8-tetrahydropyrazol o[5,1-b][1,3]oxazepine-3-carboxamide | F | 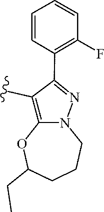 | 10.94 (s, 1H), 8.31 (d, J = 7.5 Hz, 0.5H), 8.27 (d, J = 7.7 Hz, 0.5H), 7.60 - 7.40 (m, 5H), 7.40 - 7.33 (m, 2H), 7.32 - 7.25 (m, 1H), 7.20 - 7.09 (m, 3H), 5.39 - 5.33 (m, 1H), 4.41 (d, J = 5.7 Hz, 1H), 4.31 - 4.23 (m, 1H), 4.22 - 4.13 (m, 1H), 2.20 - 2.12 (m, 1H), 2.11 -2.04 (m, 2H), 2.04 - 1.79 (m, 4H), 1.09 (t, J = 7.4 Hz, 1.5H), 1.05 (t, J = 7.5 Hz, 1.5H). | (method C) 556.3 [M+H]⁺ at 4.74 and 4.79 |
| 26 | 5-Ethyl-2-(6-methylpyridin-3-yl)-N-[(3S)-2-oxo-5-phenyl-1,3-dihydro-1,4-benzodiazepin-3-yl]-5,6,7,8-tetrahydropyrazol o[5,1-b][1,3]oxazepine-3-carboxamide | H | 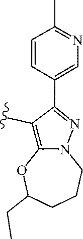 | 10.98 (s, 1H), 8.67 (dd, J = 9.8, 2.3 Hz, 1H), 8.53 (d, J = 7.6 Hz, 0.5H), 8.48 (d, J = 7.5 Hz, 0.5H), 7.95 - 7.88 (m, 1H), 7.68 - 7.61 (m, 1H), 7.55 - 7.49 (m, 1H), 7.49 - 7.41 (m, 4H), 7.35 - 7.30 (m, 2H), 7.29 - 7.23 (m, 1H), 7.23 -7.17 (m, 1H), 5.34 (d, J = 7.6 Hz, 1H), 4.46 - 4.39 (m, 1H), 4.31 -4.20 (m, 1H), 4.20 - 4.12 (m, 1H), 2.46 (s, 1H), 2.15 (d, J = 14.1 Hz, 1H), 2.10 - 1.78 (m, 6H), 1.12 -1.03 (m, 3H). | (method C) 535.4 [M+H]⁺ at 2.80 and 2.87 min |
| 27 | 5-Ethyl-N-[(3S)-9-fluoro-2-oxo-5-phenyl-1,3-dihydro-1,4-benzodiazepin-3-yl]-2-(6-methylpyridin-3-yl)-5,6,7,8-tetrahydropyrazol o[5,1-b][1,3]oxazepine-3-carboxamide | F | 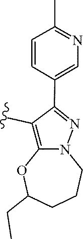 | 10.95 (s, 1H), 8.66 (dd, J= 8.3, 2.3 Hz, 1H), 8.54 (d, J= 7.5 Hz, 0.5H), 8.50 (d, J = 7.5 Hz, 0.5H), 7.95 - 7.88 (m, 1H), 7.63 - 7.56 (m, 1H), 7.56 - 7.48 (m, 3H), 7.48 - 7.42 (m, 2H), 7.35 - 7.27 (m, 1H), 7.23 - 7.18 (m, 1H), 7.18 -7.13 (m, 1H), 5.42 (d, J = 7.5 Hz, 1H), 4.46 - 4.40 (m, 1H), 4.31 -4.22 (m, 1H), 4.22 - 4.12 (m, 1H), 2.48 - 2.44 (m, 3H), 2.18 - 1.78 (m, 6H), 1.12 - 1.02 (m, 3H). | (method C) 553.3 [M+H]⁺ at 2.78 and 2.85 min |
| 30 | 4-(6-Ethylpyridin-3-yl)-N-[(3S)-9-fluoro-2-oxo-5-phenyl-2,3-dihydro-1H-1,4-benzodiazepin-3-yl]-7-oxa-2,3-diazatricyclo[6.2. 1.0^{2,6}]undeca-3,5- diene-5-carboxamide | F | 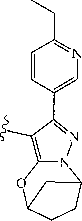 | Not available | (method C): 551.5 [M+H]⁺ at 2.41/2.46 min |
| 31 | N-[(3S)-9-fluoro-2-oxo-5-phenyl-2,3-dihydro-1H-1,4-benzodiazepin-3-yl]-4-{6-[(propan-2-yl)amino]pyridin-3-yl}-7-oxa-2,3-diazatricyclo[6.2. 1.0^{2,6}]undeca-3,5- diene-5-carboxamide | F | 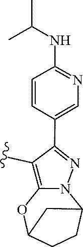 | Not available | (method C): 580.5 [M+H]⁺ at 2.38/2.42 min |

### 28. 6,6-Difluoro-N-[(3S)-9-Fluoro-2-Oxo-5-Phenyl-2,3-Dihydro-1H-1,4-Benzodiazepin-3-yl]-2-(2-Fluorophenyl)-5H,6H,7H,8H-Pyrazolo[3,2-b] [1,3]Oxazepine-3-Carboxamide

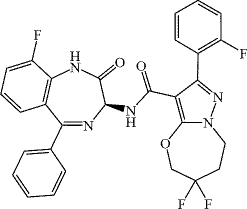 [SMILES: O=C(N[C@H]1N=C(c2ccccc2)c2cccc(F)c2NC1=O)c1c(-c2ccccc2F)nn2c1OCC(F)(F)CC2]

A solution of intermediate 29A (32 mg, 0.094 mmol) and LiOH (34 mg, 1.411 mmol) in MeOH:THF;water (1:1:1; 12 mL) was heated at 60° C. for 18 h. The reaction was cooled to rt, acidified with 1 M aq. HCl (10 mL) and the solvent removed under reduced pressure. The crude residue was dissolved in DMF (5 mL), NEt₃ (66 µL, 0.470 mmol) and (3S)-3-amino-9-fluoro-5-phenyl-1,3-dihydro-1,4-benzodiazepin-2-one (25 mg, 0.094 mmol) added, followed by HATU (36 mg, 0.094 mmol) and the reaction stirred at rt for 1 h. The reaction was diluted with water (20 mL) and extracted with EtOAc (2× 15 mL). The combined organic layers were washed with brine (20 mL), passed through a phase separation cartridge and the solvent removed under reduced pressure. Purification by flash chromatography [0-100% (10% MeOH in EtOAc) in heptane] afforded a white solid (30 mg, 57%). LCMS (method C): m/z 564.3 [M+H]⁺ at 4.44 min.¹H NMR (500 MHz, DMSO-d₆) δ 10.97 (s, 1H), 8.27 (d, J = 7.6 Hz, 1H), 7.62 -7.38 (m, 8H), 7.31 (td, J = 8.1, 4.9 Hz, 1H), 7.24 - 7.12 (m, 3H), 5.37 (d, J = 7.5 Hz, 1H), 4.72 (t, J= 10.9 Hz, 2H), 4.44 - 4.38 (m, 2H), 2.60 (d, J = 9.3 Hz, 2H).

### 29. 2-Fluorophenyl)-6-Morpholin-4-yl-N-[(3R)-9-Fluoro-2-Oxo-5-Phenyl-1,3-Dihydro-1,4-Benzodiazepin-3-yl]-5,6,7,8-Tetrahydropyrazolo[5,1-b][1,3]Oxazepine-3-Carboxamide

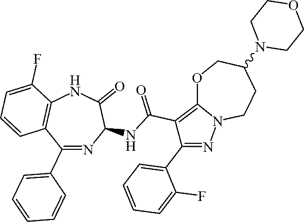 [SMILES: O=C(N[C@H]1N=C(c2ccccc2)c2cccc(F)c2NC1=O)c1c(-c2ccccc2F)nn2c1OCC(N1CCOCC1)CC2]

Prepared by an analogous procedure to that described for Example 28 with additional purification by HPLC [HPLC Method 4: 10-100% MeCN in water (0.1% formic acid)]. Mixture of diastereomers. LCMS (method C): m/z 613.5 [M+H]⁺ at 2.36 and 2.47 min.

### 32. 4-Fluoro-4-Methylsulfonylphenyl)-N-[(3S)-9-Fluoro-2-Oxo-5-Phenyl-1,3-Dihydro-1,4-Benzodiazepin-3-yl]-7-Oxa-2,3-Diazatricyclo[6.2.1.02,6]Undeca-3,5-Diene-5-Carboxamide

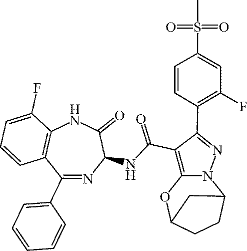 [SMILES: CS(=O)(=O)c1ccc(-c2nn3c(c2C(=O)N[C@H]2N=C(c4ccccc4)c4cccc(F)c4NC2=O)OC2CCC3C2)c(F)c1]

A solution of intermediate 45A (121 mg, 0.33 mmol), DIPEA (115 µL, 0.661 mmol) and HATU (138 mg, 0.363 mmol) in DMF (3 mL) was stirred for 10 min at rt. (3S)-3-Amino-9-fluoro-5-phenyl-1,3-dihydro-1,4-benzodiazepin-2-one (89 mg, 0.33 mmol) was added and the reaction stirred at rt for 22 h. The reaction was quenched with water (15 mL) and the resultant precipitate collected by filtration, washing with water (2×10 mL). The precipitate was dissolved in CH₂Cl₂, the solvent removed under reduced pressure and the residue purified by flash chromatography (60-100% EtOAc in heptane) to afford a white solid (173 mg, 85%). ¹H NMR (400 MHz, DMSO-d₆) δ 10.97 (s, 0.5H), 10.96 (s, 0.5H), 7.88 - 7.80 (m, 1H), 7.79 - 7.64 (m, 3H), 7.64 - 7.39 (m, 6H), 7.36 - 7.25 (m, 1H), 7.19 - 7.06 (m, 1H), 5.54 - 5.42 (m, 1H), 5.34 (d, J = 7.5 Hz, 1H), 4.92 - 4.78 (m, 1H), 3.28 (s, 3H), 2.49 - 2.41 (m, 4H), 2.31 - 2.04 (m, 5H). LRMS (APCI+) m/z 618.4 [M+H]⁺.

The following compounds of the invention were prepared by chiral resolution from the denoted parent mixture of diastereomers by the method indicated and were isolated as a single stereoisomer of unknown absolute stereochemistry. The retention time for each resolved diastereomer under the indicated analytical conditions is denoted by t_{R}. The stereochemical configuration of these compounds has been designated as R* or S*, with the arbitrarily defined stereocenter marked by an asterisk.

TABLE 7

Resolution of Diastereomers

| Parent Example | Preparative Resolution Method (Mobile Phase) | Analysis Method (Mobile Phase) | Diastereomer 1 Example t_{R} (min) | Diastereomer 2 Example t_{R}(min) |
|---|---|---|---|---|
| 10 | HPLC Method 2 60% MeCN in water (0.1% formic acid) | HPLC Method 1A 60% MeCN in water (0.1% formic acid) | 10-1 t_{R} = 5.91 min | 10-2 t_{R} = 9.92 min |
| 11 | HPLC Method 1 43% MeCN in water (0.1% formic acid) | LCMS Method A | 11-1 t_{R} = 1.51 min | 11-2 t_{R} = 1.53 min |
| 12 | HPLC Method 2 60% MeCN in water (0.1% formic acid) | HPLC Method 1A 60% MeCN in water (0.1% formic acid) | 12-1 t_{R} = 5.94 min | 12-2 t_{R} = 10.2 min |
| 13 | HPLC Method 3 30-60% MeCN in water (0.3% ammonia) over 10 min | LCMS Method A | 13-1 t_{R} = 1.58 min | 13-2 t_{R} = 1.61 min |
| 14 | SFC Method 2 35% MeCN/MeOH (1:1) | SFC Method 2A 40% MeCN/MeOH (1:1) | 14-1 t_{R} = 2.24 min | 14-2 t_{R} = 3.07 min |
| 15 | SFC Method 2 35% MeOH | SFC Method 2A 45% MeOH | 15-1 t_{R} = 2.42 min | 15-2 t_{R} = 2.96 min |
| 16 | SFC Method 3 30% MeOH | SFC Method 3A 40% MeOH (0.1% Ammonia) | 16-1 t_{R} = 3.41 min | 16-2 t_{R} = 3.96 min |
| 17 | SFC Method 1 30% iPrOH | SFC Method 1A 40% iPrOH (0.1% ammonia) | 17-1 t_{R} = 3.24 min | 17-2 t_{R} = 4.01 min |
| 19 | SFC Method 1 55% EtOH (0.1% ammonia) | SFC Method 4A 60% EtOH (0.1% ammonia) | 19-1 t_{R}= 1.74 min | 19-2 t_{R}= 2.20 min |
| 24 | SFC Method 2: 40% EtOH (0.1% ammonia) | SFC Method 2A: 50% EtOH (0.1% formic acid) | 24-1 t_{R} = 3.23 min | 24-2 t_{R} = 3.90 min |
| 25 | SFC Method 2: 55% EtOH (0.1% ammonia) | SFC Method 2A: 50% EtOH (0.1% ammonia) | 25-1 t_{R} = 2.93 min | 25-2 t_{R} = 4.17 min |
| 26 | HPLC Method 2: 70% MeCN in water (0.1% ammonia) | HPLC Method 1A: 90% MeCN in water (0.1% ammonia) | 26-1 t_{R} = 4.45 min | 26-2 t_{R} = 5.95 min |
| 27 | HPLC Method 2: 70% MeCN with water (0.1% ammonia) | HPLC Method 1A: 90% MeCN in water (0.1% ammonia) | 27-1 t_{R} = 4.78 min | 27-2 t_{R} = 7.39 min |
| 30 | SFC Method 1 55% EtOH (0.1% ammonia) | SFC Method 5A 60% EtOH (0.1% ammonia) | 30-1 t_{R} = 1.98 min | 30-2 t_{R} = 2.44 min |
| 31 | SFC Method 1 50% EtOH (0.1% ammonia) | SFC Method 4A 60% MeOH (0.1% ammonia) | 31-1 t_{R}= 2.03 min | 31-2 t_{R}= 2.42 min |

### Table 8 - Analytical Data for Resolved Example Compounds

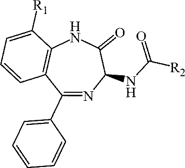

| Example | Name | R¹ | R² | ¹H NMR δ (500 MHz, DMSO-d₆) | LCMS ES+ m/z |
|---|---|---|---|---|---|
| 10-1 | (5R*)-2-[6-(ethylamino)-2-fluoropyridin-3 -yl] -5-methyl-N-[(3S)-2-oxo-5-phenyl-1,3-dihydro-1,4-benzodiazepin-3-yl]-5,6,7,8-tetrahydropyrazolo [ 5,1-b][1,3]oxazepine-3-carboxamide | H | 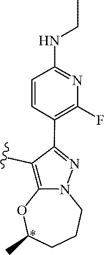 | δ 10.97 (s, 1H), 8.35 (d, J = 7.6 Hz, 1H), 7.67 - 7.60 (m, 1H), 7.54 - 7.47 (m, 1H), 7.49 -7.39 (m, 5H), 7.34 - 7.28 (m, 2H), 7.28 - 7.22 (m, 1H), 6.97 - 6.91 (m, 1H), 6.27 (dd, J = 8.3, 1.8 Hz, 1H), 5.28 (d, J = 7.5 Hz, 1H), 4.44 - 4.32 (m, 2H), 4.18 - 4.09 (m, 1H), 3.23 - 3.14 (m, 2H), 2.17 - 2.11 (m, 1H), 2.04 - 1.90 (m, 2H), 1.85 - 1.74 (m, 1H), 1.57 (d, J = 6.3 Hz, 3H), 1.11 (t, J = 7.2 Hz, 3H). | (method C) 568.4 [M+H]⁺ at 4.29 min |
| 10-2 | (5S*)-2-[6-(ethylamino)-2-fluoropyridin-3 -yl]-5-methyl-N-[(3S)-2-oxo-5-phenyl-1,3-dihydro-1,4-benzodiazepin-3-yl]-5,6,7,8-tetrahydropyrazolo [ 5,1-b][1,3]oxazepine-3-carboxamide | H | 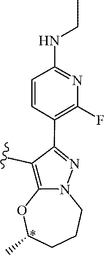 | δ 10.98 (s, 1H), 8.29 (d, J = 8.0 Hz, 1H), 7.67 - 7.60 (m, 1H), 7.54 - 7.47 (m, 1H), 7.47 -7.39 (m, 5H), 7.34 - 7.28 (m, 2H), 7.28 - 7.22 (m, 1H), 6.96 - 6.91 (m, 1H), 6.27 (dd, J = 8.3, 1.8 Hz, 1H), 5.32 (d, J = 7.9 Hz, 1H), 4.41 - 4.31 (m, 2H), 4.17 - 4.08 (m, 1H), 3.23 - 3.14 (m, 2H), 2.17 - 2.11 (m, 1H), 2.04 - 1.98 (m, 1H), 2.00 - 1.88 (m, 1H), 1.85 - 1.74 (m, 1H), 1.64 (d, J = 6.2 Hz, 3H), 1.10 (t, J = 7.2 Hz, 3H). | (method C) 568.4 [M+H]⁺ at 4.41 min |
| 11-1 | (5R)-2-[6-(ethylamino)-2-fluoropyridin-3 -yl] -N-[(3S)-9-fluoro-2-oxo-5-phenyl-1,3-dihydro-1,4-benzodiazepin-3-yl]-5-methyl-5,6,7,8-tetrahydropyrazolo [ 5,1-b][1,3]oxazepine-3-carboxamide | F | 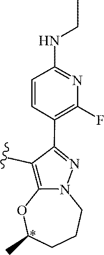 | δ 10.94 (s, 1H), 8.38 (d, J = 7.4 Hz, 1H), 7.61 - 7.52 (m, 1H), 7.55 - 7.48 (m, 1H), 7.51 -7.43 (m, 4H), 7.45 - 7.39 (m, 1H), 7.32 - 7.26 (m, 1H), 7.16 - 7.11 (m, 1H), 6.97 - 6.91 (m, 1H), 6.27 (dd, J = 8.3, 1.8 Hz, 1H), 5.35 (d, J = 7.5 Hz, 1H), 4.45 - 4.33 (m, 2H), 4.18 -4.09 (m, 1H), 3.24 - 3.15 (m, 2H), 2.17 - 2.10 (m, 1H), 2.04 - 1.90 (m, 2H), 1.85 - 1.74 (m, 1H), 1.58 (d, J = 6.3 Hz, 3H), 1.11 (t, J = 7.2 Hz, 3H). | (method C) 586.4 [M+H]⁺ at 4.37 min |
| 11-2 | (5S*)-2-[6-(ethylamino)-2-fluoropyridin-3 -yl] -N-[(3S)-9-fluoro-2-oxo-5-phenyl-1,3-dihydro-1,4-benzodiazepin-3-yl]-5-methyl-5,6,7,8-tetrahydropyrazolo [ 5,1-b][1,3]oxazepine-3-carboxamide | F | 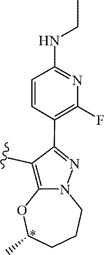 | δ 10.96 (s, 1H), 8.31 (d, J = 7.9 Hz, 1H), 7.61 - 7.55 (m, 1H), 7.55 - 7.50 (m, 1H), 7.50 -7.40 (m, 5H), 7.32 - 7.28 (m, 1H), 7.16 - 7.11 (m, 1H), 6.97 - 6.91 (m, 1H), 6.27 (dd, J = 8.2, 1.8 Hz, 1H), 5.39 (d, J = 7.8 Hz, 1H), 4.41 - 4.31 (m, 2H), 4.17 - 4.08 (m, 1H), 3.23 - 3.14 (m, 2H), 2.17 - 2.11 (m, 1H), 2.05 - 1.89 (m, 2H), 1.85 - 1.74 (m, 1H), 1.65 (d, J = 6.2 Hz, 3H), 1.10 (t, J = 7.2 Hz, 3H). | (method C) 586.4 [M+H]⁺ at 4.48 min |
| 12-1 | (5R*)-2-[2-fluoro-6-(propan-2-ylamino)pyridin-3-yl]-5-methyl-N-[(3S)-2-oxo-5-phenyl-1,3-dihydro-1,4-benzodiazepin-3-yl]-5,6,7,8-tetrahydropyrazolo [ 5,1-b][1,3]oxazepine-3-carboxamide | H | 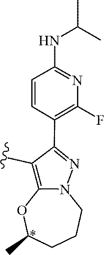 | δ 10.97 (s, 1H), 8.36 (d, J = 7.6 Hz, 1H), 7.67 - 7.60 (m, 1H), 7.55 - 7.47 (m, 1H), 7.47 -7.37 (m, 5H), 7.34 - 7.28 (m, 2H), 7.28 - 7.22 (m, 1H), 6.83 (d, J = 7.6 Hz, 1H), 6.26 (dd, J = 8.3, 1.8 Hz, 1H), 5.28 (d, J = 7.6 Hz, 1H), 4.45 - 4.32 (m, 2H), 4.18 - 4.09 (m, 1H), 3.92 - 3.82 (m, 1H), 2.17 - 2.11 (m, 1H), 2.04 - 1.90 (m, 2H), 1.85 - 1.74 (m, 1H), 1.57 (d, J = 6.3 Hz, 3H), 1.14 - 1.10 (d, J = 6.4 Hz, 6H). | (method C) 582.4 [M+H]⁺ at 4.51 min |
| 12-2 | (5S*)-2-[2-fluoro-6-(propan-2-ylamino)pyridin-3-yl]-5-methyl-N-[(3S)-2-oxo-5-phenyl-1,3-dihydro-1,4-benzodiazepin-3-yl]-5,6,7,8-tetrahydropyrazolo [ 5,1-b][1,3]oxazepine-3-carboxamide | H | 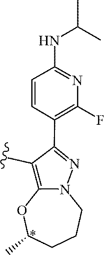 | δ 10.98 (s, 1H), 8.29 (d, J = 8.0 Hz, 1H), 7.67 - 7.60 (m, 1H), 7.54 - 7.47 (m, 1H), 7.47 -7.37 (m, 5H), 7.34 - 7.28 (m, 2H), 7.28 - 7.22 (m, 1H), 6.82 (d, J = 7.6 Hz, 1H), 6.26 (dd, J = 8.3, 1.8 Hz, 1H), 5.32 (d, J = 7.9 Hz, 1H), 4.40 - 4.30 (m, 2H), 4.17 - 4.08 (m, 1H), 3.92 - 3.82 (m, 1H), 2.17 - 2.11 (m, 1H), 2.04 - 1.89 (m, 2H), 1.85 - 1.74 (m, 1H), 1.64 (d, J = 6.3 Hz, 3H), 1.12 (d, J = 6.5 Hz, 6H). | (method C) 582.4 [M+H]⁺ at 4.63 min |
| 13-1 | (5R*)-N-[(3S)-9-fluoro-2-oxo-5-phenyl-1,3-dihydro-1,4-benzodiazepin-3-yl]-2-[2-fluoro-6-(propan-2-ylamino)pyridin-3-yl]-5-methyl-5,6,7,8-tetrahydropyrazolo [ 5,1-b][1,3]oxazepine-3-carboxamide | F | 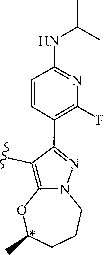 | δ 10.94 (s, 1H), 8.38 (d, J = 7.5 Hz, 1H), 7.62 - 7.54 (m, 1H), 7.54 - 7.50 (m, 1H), 7.50 -7.43 (m, 4H), 7.43 - 7.38 (m, 1H), 7.34 - 7.28 (m, 1H), 7.13 (d, J = 7.9 Hz, 1H), 6.83 (d, J = 7.6 Hz, 1H), 6.26 (dd, J = 8.3, 1.8 Hz, 1H), 5.36 (d, J = 7.5 Hz, 1H), 4.45 - 4.33 (m, 2H), 4.18 - 4.09 (m, 1H), 3.92 -3.82 (m, 1H), 2.17 - 2.11 (m, 1H), 2.04 - 1.90 (m, 2H), 1.85 - 1.74 (m, 1H), 1.57 (d, J = 6.3 Hz, 3H), 1.12 (d, J = 6.5 Hz, 6H). | (method C) 600.4 [M+H]⁺ at 4.71 min |
| 13-2 | (5S*)-N-[(3S)-9-fluoro-2-oxo-5-phenyl-1,3-dihydro-1,4-benzodiazepin-3-yl]-2-[2-fluoro-6-(propan-2-ylamino)pyridin-3-yl]-5-methyl-5,6,7,8-tetrahydropyrazolo [ 5,1-b][1,3]oxazepine-3-carboxamide | F | 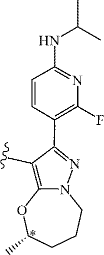 | δ 10.95 (s, 1H), 8.31 (d, J = 7.9 Hz, 1H), 7.62 - 7.55 (m, 1H), 7.55 - 7.50 (m, 1H), 7.50 -7.44 (m, 4H), 7.44 - 7.38 (m, 1H), 7.34 - 7.27 (m, 1H), 7.14 (d, J = 7.9 Hz, 1H), 6.82 (d, J = 7.6 Hz, 1H), 6.26 (dd, J = 8.2, 1.8 Hz, 1H), 5.40 (d, J = 7.9 Hz, 1H), 4.39 - 4.32 (m, 2H), 4.17 - 4.08 (m, 1H), 3.92 -3.82 (m, 1H), 2.17 - 2.11 (m, 1H), 2.04 - 1.89 (m, 2H), 1.85 - 1.74 (m, 1H), 1.65 (d, J = 6.3 Hz, 3H), 1.12 (d, J = 6.4 Hz, 6H). | (method C) 600.4 [M+H]⁺ at 4.83 min |
| 14-1 | (8R*)-2-(2-fluorophenyl)-8-methyl-N-(2-oxo-5-phenyl-1,3-dihydro-1,4-benzodiazepin-3-yl)-5,6,7,8-tetrahydropyrazolo [ 5,1-b][1,3]oxazepine-3-carboxamide | H | 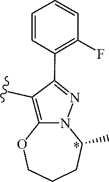 | δ 11.00 (s, 1H), 8.33 (d, J = 7.6 Hz, 1H), 7.64 (td, J = 7.6, 6.8, 1.7 Hz, 1H), 7.51 (m, J = 8.7, 5.8, 3.4 Hz, 1H), 7.45 (d, J = 3.3 Hz, 4H), 7.39 (m, J = 7.3, 2.2 Hz, 2H), 7.34 - 7.28 (m, 2H), 7.25 (t, J = 7.5 Hz, 1H), 7.21 - 7.10 (m, 2H), 5.27 (d, J = 7.6 Hz, 1H), 4.69 (t, J = 6.7 Hz, 1H), 4.56 - 4.50 (m, 1H), 4.34 (dd, J = 11.4, 8.8 Hz, 1H), 2.31 (s, 1H), 2.04 (d, J = 11.5 Hz, 2H), 1.92 (d, J = 14.3 Hz, 1H), 1.49 (d, J = 6.9 Hz, 3H). | (method C) 524.4 [M+H]⁺ at 4.29 min |
| 14-2 | (8S*)-2-(2-fluorophenyl)-8-methyl-N-(2-oxo-5-phenyl-1,3-dihydro-1,4-benzodiazepin-3-yl)-5,6,7,8-tetrahydropyrazolo [ 5,1-b][1,3]oxazepine-3-carboxamide | H | 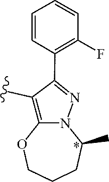 | δ 11.00 (s, 1H), 8.37 (d, J = 7.7 Hz, 1H), 7.63 (m, J = 8.5, 7.1, 1.7 Hz, 1H), 7.52 (tt, J = 5.9, 2.6 Hz, 1H), 7.51 - 7.41 (m, 4H), 7.39 (m, J = 7.5, 4.4, 2.1 Hz, 2H), 7.30 (m, J = 7.9, 1.9 Hz, 2H), 7.28 - 7.22 (m, 1H), 7.21 - 7.10 (m, 2H), 5.27 (d, J = 7.7 Hz, 1H), 4.74 - 4.67 (m, 1H), 4.62 - 4.55 (m, 1H), 4.31 - 4.23 (m, 1H), 2.34 (d, J = 13.0 Hz, 1H), 2.05 (td, J = 10.2, 5.2 Hz, 2H), 1.94 (d, J= 14.4 Hz, 1H), 1.49 (d, J = 6.9 Hz, 3H). | (method C) 524.4 [M+H]⁺ at 4.38 min |
| 15-1 | (8R*)-N-(9-fluoro-2-oxo-5-phenyl-1,3-dihydro-1,4-benzodiazepin-3-yl)-2-(2-fluorophenyl)-8-methyl-5,6,7,8-tetrahydropyrazolo [ 5,1-b][1,3]oxazepine-3-carboxamide | F | 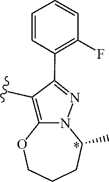 | δ 10.97 (s, 1H), 8.34 (d, J = 7.5 Hz, 1H), 7.62 - 7.43 (m, 6H), 7.43 - 7.35 (m, 2H), 7.30 (td, J = 8.1, 4.9 Hz, 1H), 7.21 -7.10 (m, 3H), 5.35 (d, J = 7.4 Hz, 1H), 4.69 (m, J = 6.7, 2.8 Hz, 1H), 4.56 - 4.49 (m, 1H), 4.34 (m, J = 9.5, 8.8, 4.9 Hz, 1H), 2.31 (s, 1H), 2.06 (m, J = 11.9, 3.6 Hz, 2H), 1.91 (d, J = 13.0 Hz, 1H), 1.49 (d, J = 6.9 Hz, 3H). | (method C) 542.4 [M+H]⁺ at 4.45 min |
| 15-2 | (8S*)-N-(9-fluoro-2-oxo-5-phenyl-1,3-dihydro-1,4-benzodiazepin-3-yl)-2-(2-fluorophenyl)-8-methyl-5,6,7,8-tetrahydropyrazolo [ 5,1-b][1,3]oxazepine-3-carboxamide | F | 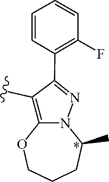 | δ 10.96 (s, 1H), 8.39 (d, J = 7.6 Hz, 1H), 7.61 - 7.39 (m, 6H), 7.42 - 7.35 (m, 2H), 7.34 -7.26 (m, 1H), 7.21 - 7.13 (m, 2H), 7.13 (t, J = 4.4 Hz, 1H), 5.35 (d, J = 7.7 Hz, 1H), 4.74 -4.67 (m, 1H), 4.60 (d, J = 12.2 Hz, 1H), 4.27 (t, J = 10.3 Hz, 1H), 2.32 (d, J = 12.8 Hz, 1H), 2.05 (t, J = 11.9 Hz, 2H), 1.95 (s, 1H), 1.49 (d, J = 6.9 Hz, 3H). | (method C) 542.4 [M+H]⁺ at 4.53 min |
| 16-1 | (5R*)-2-(2,4-difluorophenyl)-5-methyl-N-[(3S)-2-oxo-5-phenyl-1,3-dihydro-1,4-benzodiazepin-3-yl]-5,6,7,8-tetrahydropyrazolo [ 5,1-b][1,3]oxazepine-3-carboxamide | H | 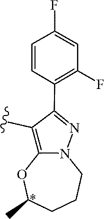 | δ 10.99 (s, 1H), 8.32 (d, J = 8.0 Hz, 1H), 7.64 (t, J = 7.6 Hz, 1H), 7.55 -7.48 (m, 1H), 7.48 -7.38 (m, 5H), 7.31 (d, J = 8.2 Hz, 2H), 7.26 (t, J = 7.5 Hz, 1H), 7.22 -7.14 (m, 1H), 7.09 -7.02 (m, 1H), 5.31 (d, J = 7.9 Hz, 1H), 4.41 (q, J = 8.3, 7.1 Hz, 2H), 4.18 (t, J = 12.6 Hz, 1H), 2.16 (d, J = 14.6 Hz, 1H), 2.07 - 1.92 (m, 2H), 1.83 (q, J = 12.3 Hz, 1H), 1.66 (d, J = 6.2 Hz, 3H). | (method C) 542.4 [M+H]⁺ at 4.67 min |
| 16-2 | (5S*)-2-(2,4-difluorophenyl)-5-methyl-N-[(3S)-2-oxo-5-phenyl-1,3-dihydro-1,4-benzodiazepin-3-yl]-5,6,7,8-tetrahydropyrazolo [ 5,1-b][1,3]oxazepine-3-carboxamide | H | 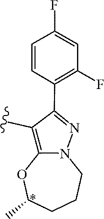 | δ 10.97 (s, 1H), 8.38 (d, J = 7.5 Hz, 1H), 7.67 -7.60 (m, 1H), 7.51 (q, J = 4.6 Hz, 1H), 7.48 -7.38 (m, 5H), 7.34 -7.28 (m, 2H), 7.25 (t, J = 7.5 Hz, 1H), 7.22 -7.14 (m, 1H), 7.10 -7.02 (m, 1H), 5.27 (d, J = 7.4 Hz, 1H), 4.46 (t, J = 8.0 Hz, 1H), 4.40 (d, J = 14.9 Hz, 1H), 4.19 (t, J = 12.7 Hz, 1H), 2.17 (d, J = 14.7 Hz, 1H), 2.07 -1.93 (m, 2H), 1.83 (q, J = 11.9, 11.4 Hz, 1H), 1.59 (d, J = 6.3 Hz, 3H). | (method C) 542.4 [M+H]⁺ at 4.53 min |
| 17-1 | (5R*)-2-(2,4-difluorophenyl)-N-[(3S)-9-fluoro-2-oxo-5-phenyl-1,3-dihydro-1,4-benzodiazepin-3-yl]-5-methyl-5,6,7,8-tetrahydropyrazolo [ 5,1-b][1,3]oxazepine-3-carboxamide | F | 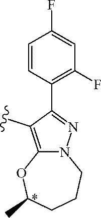 | δ 10.95 (s, 1H), 8.40 (d, J = 7.5 Hz, 1H), 7.58 (t, J = 9.2 Hz, 1H), 7.56 -7.50 (m, 1H), 7.50 -7.45 (m, 3H), 7.47 - 7.38 (m, 2H), 7.33 -7.26 (m, 1H), 7.23 -7.15 (m, 1H), 7.13 (d, J = 7.9 Hz, 1H), 7.10 -7.03 (m, 1H), 5.36 (d, J = 7.4 Hz, 1H), 4.50 -4.37 (m, 2H), 4.23 - 4.15 (m, 1H), 3.81 - 3.72 (m, OH), 2.20 -2.13 (m, 1H), 2.07 - 1.93 (m, 2H), 1.83 (q, J = 12.3 Hz, 1H), 1.59 (d, J = 6.3 Hz, 3H). | (method C) 560.3 [M+H]⁺ at 4.62 min |
| 17-2 | (5S*)-2-(2,4-difluorophenyl)-N-[(3S)-9-fluoro-2-oxo-5-phenyl-1,3-dihydro-1,4-benzodiazepin-3-yl]-5-methyl-5,6,7,8-tetrahydropyrazolo [ 5,1-b][1,3]oxazepine-3-carboxamide | F | 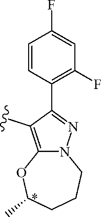 | δ 10.97 (s, 1H), 8.34 (d, J = 7.9 Hz, 1H), 7.58 (t, J = 9.2 Hz, 1H), 7.53 (t, J = 7.0 Hz, 1H), 7.50 -7.43 (m, 4H), 7.45 -7.38 (m, 1H), 7.31 (q, J = 7.5 Hz, 1H), 7.23 -7.15 (m, 1H), 7.15 (d, J = 7.9 Hz, 1H), 7.10 -7.02 (m, 1H), 5.40 (d, J = 7.8 Hz, 1H), 4.45 - 4.37 (m, 2H), 4.34 (d, J = 4.2 Hz, OH), 4.18 (t, J = 12.7 Hz, 1H), 2.17 (d, J = 14.5 Hz, 1H), 2.07 -1.92 (m, 2H), 1.83 (q, J = 12.4 Hz, 1H), 1.67 (d, J = 6.2 Hz, 3H). | (method C) 560.3 [M+H]⁺ at 4.74 min |
| 19-1 | (1S*,8R*)-N-[(3S)-9-fluoro-2-oxo-5-phenyl-2,3-dihydro-1H-1,4-benzodiazepin-3-yl]-4-(2-fluorophenyl)-7-oxa-2,3-diazatricyclo[6.2.1. 0^{2,6}]undeca-3,5- diene-5-carboxamide | F | 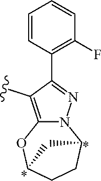 | δ 10.93 (s, 1H), 7.80 (d, J = 7.6 Hz, 1H), 7.61-7.53 (m, 1H), 7.56 -7.50 (m, 1H), 7.50 -7.42 (m, 5H), 7.42 -7.34 (m, 2H), 7.31-7.26 (m, 1H), 7.20 -7.14 (m, 1H), 7.14 - 7.11 (m, 1H), 7.14 -7.09 (m, 1H), 5.48 -5.45 (m, 1H), 5.34 (d, J = 7.6 Hz, 1H), 4.84 (s, 1H), 2.47 -2.40 (m, 1H), 2.29 - 2.20 (m, 2H), 2.22 - 2.03 (m, 2H). | (method C) 540.4 [M+H]⁺ at 3.95 |
| 19-2 | (1R*,8S*)-N-[(3S)-9-fluoro-2-oxo-5-phenyl-2,3-dihydro-1H-1,4-benzodiazepin-3-yl]-4-(2-fluorophenyl)-7-oxa-2,3-diazatricyclo[6.2.1. 0^{2,6}]undeca-3,5- diene-5-carboxamide | F | 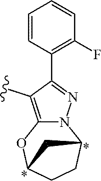 | δ 10.95 (s, 1H), 7.79 (d, J = 7.6 Hz, 1H), 7.60 -7.50 (m, 2H), 7.50 -7.44 (m, 3H), 7.47 -7.42 (m, 1H), 7.42 -7.33 (m, 2H), 7.30 -7.26 (m, 1H), 7.20 -7.13 (m, 1H), 7.16 - 7.09 (m, 2H), 5.46 (s, 1H), 5.33 (d, J = 7.5 Hz, 1H), 4.84 (s, 1H), 2.47 -2.40 (m, 1H), 2.26 - 2.14 (m, 4H), 2.13 - 2.04 (m, 1H). | (method C) 540.4 [M+H]⁺ at 3.98 |
| 24-1 | (5R*)-5-ethyl-2-(2-fluorophenyl)-N-[(3S)-2-oxo-5-phenyl-2,3-dihydro-1H-1,4-benzodiazepin-3-yl]-5H,6H,7H,8H-pyrazolo[3,2-b][1,3]oxazepine-3-Carboxamide | H | 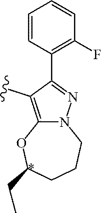 | δ 10.98 (s, 1H), 8.25 (d, J = 7.8 Hz, 1H), 7.67 -7.60 (m, 1H), 7.52 (ddd, J = 8.8, 5.9, 2.7 Hz, 1H), 7.45 (d, J = 6.0 Hz, 4H), 7.45 -7.35 (m, 2H), 7.31 (dd, J = 7.9, 2.2 Hz, 2H), 7.25 (t, J = 7.6 Hz, 1H), 7.21 -7.10 (m, 2H), 5.29 (d, J = 7.8 Hz, 1H), 4.46 - 4.39 (m, 1H), 4.28 (dd, J = 10.6, 5.4 Hz, 1H), 4.22 -4.14 (m, 1H), 2.18 (d, J = 15.1 Hz, 1H), 2.11 (dt, J = 12.7, 6.3 Hz, 1H), 1.96 (dt, J = 13.9, 6.8 Hz, 2H), 1.90 - 1.82 (m, 1H), 1.06 (t, J = 3.7 Hz, 3H). | LCMS (method C) 538.3 [M+H]⁺ at 4.81 min |
| 24-2 | (5S*)-5-ethyl-2-(2-fluorophenyl)-N-[(3S)-2-oxo-5-phenyl-2,3-dihydro-1H-1,4-benzodiazepin-3-yl]-5H,6H,7H,8H-pyrazolo[3,2-b][1,3]oxazepine-3-Carboxamide | H | 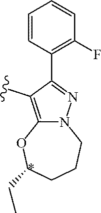 | δ 10.96 (s, 1H), 8.29 (d, J = 7.8 Hz, 1H), 7.64 (t, J = 7.7 Hz, 1H), 7.51 (m, J = 6.1, 2.7 Hz, 1H), 7.45 (d, J = 6.7 Hz, 4H), 7.38 (m, J = 7.7, 6.1 Hz, 2H), 7.33 -7.28 (m, 2H), 7.25 (t, J = 7.6 Hz, 1H), 7.20 -7.09 (m, 2H), 5.29 (d, J = 7.7 Hz, 1H), 4.43 (d, J = 14.2 Hz, 1H), 4.28 (m, J = 10.9, 5.9 Hz, 1H), 4.18 (t, J = 12.5 Hz, 1H), 2.17 (d, J = 14.4 Hz, 1H), 2.08 (d, J = 13.5 Hz, 1H), 1.99 (m, J = 15.4, 9.4, 7.7 Hz, 2H), 1.86 (m, J = 14.1, 8.2 Hz, 2H), 1.10 (t, J = 7.4 Hz, 3H). | (method C) 538.3 [M+H]⁺ at 4.73 min |
| 25-1 | (5R*)-5-ethyl-2-(2-fluorophenyl)-N-[(3S)-9-fluoro-2-oxo-5-phenyl-1,3-dihydro-1,4-benzodiazepin-3-yl]-5,6,7,8-tetrahydropyrazolo [ 5,1-b][1,3]oxazepine-3-carboxamide | F | 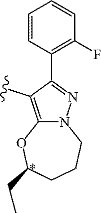 | δ 10.96 (s, 1H), 8.27 (d, J = 7.8 Hz, 1H), 7.61 -7.55 (m, 1H), 7.55 -7.51 (m, 1H), 7.51 -7.43 (m, 5H), 7.42 -7.35 (m, 2H), 7.33 -7.26 (m, 1H), 7.20 -7.11 (m, 3H), 5.38 (d, J = 7.7 Hz, 1H), 4.46 - 4.39 (m, 1H), 4.30 -4.26 (m, 1H), 4.22 -4.14 (m, 1H), 2.21 -2.15 (m, 1H), 2.15 - 2.05 (m, 1H), 2.03 - 1.92 (m, 2H), 1.91 -1.80 (m, 1H), 1.06 (t, J = 7.5 Hz, 3H). | (method C) 556.4 [M+H]⁺ at 4.88 min |
| 25-2 | (5S*)-5-ethyl-2-(2-fluorophenyl)-N-[(3S)-9-fluoro-2-oxo-5-phenyl-1,3-dihydro-1,4-benzodiazepin-3-yl]-5,6,7,8-tetrahydropyrazolo [ 5,1-b][1,3]oxazepine-3-carboxamide | F | 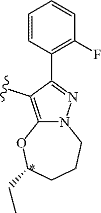 | δ 10.94 (s, 1H), 8.32 (d, J = 7.7 Hz, 1H), 7.62 -7.41 (m, 6H), 7.41-7.25 (m, 3H), 7.21 -7.08 (m, 3H), 5.37 (d, J = 7.7 Hz, 1H), 4.43 (dd, J = 14.3, 4.9 Hz, 1H), 4.32 - 4.24 (m, 1H), 4.18 (dd, J = 13.7, 11.1 Hz, 1H), 2.23 - 1.78 (m, 6H), 1.10 (t, J= 7.4 Hz, 3H). | (method C) 556.4 [M+H]⁺ at 4.80 min |
| 26-1 | (5R*)-5-ethyl-2-(6-methylpyridin-3-yl)-N-[(3S)-2-oxo-5-phenyl-2,3-dihydro-1H-1,4-benzodiazepin-3-yl]-5H,6H,7H,8H-pyrazolo[3,2-b][1,3]oxazepine-3-carboxamide | H | 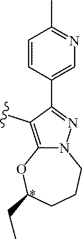 | δ 10.99 (s, 1H), 8.68 (d, J = 2.3 Hz, 1H), 8.54 (d, J = 7.6 Hz, 1H), 7.93 (dd, J = 8.0, 2.3 Hz, 1H), 7.65 (ddd, J = 8.6, 7.1, 1.6 Hz, 1H), 7.57 -7.41 (m, 6H), 7.36 -7.30 (m, 2H), 7.27 (td, J = 7.5, 1.2 Hz, 1H), 7.21 (d, J = 8.1 Hz, 1H), 5.34 (d, J = 7.5 Hz, 1H), 4.43 (dd, J = 13.6, 4.9 Hz, 1H), 4.28 (dt, J = 10.9, 6.0 Hz, 1H), 4.21 - 4.12 (m, 1H), 2.47 (s, 3H), 2.16 (d, J = 14.7 Hz, 1H), 2.07 (dt, J = 13.0, 4.0 Hz, 1H), 2.01 -1.88 (m, 2H), 1.87 - 1.77 (m, 1H), 1.06 (t, J = 7.4 Hz, 3H). | (method C) 535.4 [M+H]⁺ at 2.90 min |
| 26-2 | (5S*)-5-ethyl-2-(6-methylpyridin-3-yl)-N-[(3S)-2-oxo-5-phenyl-2,3-dihydro-1H-1,4-benzodiazepin-3-yl]-5H,6H,7H,8H-pyrazolo[3,2-b][1,3]oxazepine-3-carboxamide | H | 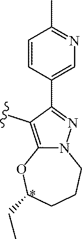 | δ 10.97 (s, 1H), 8.66 (d, J = 2.2 Hz, 1H), 8.49 (d, J = 7.6 Hz, 1H), 7.91 (dd, J = 8.0, 2.3 Hz, 1H), 7.65 (ddd, J = 8.4, 7.2, 1.7 Hz, 1H), 7.56 -7.42 (m, 6H), 7.33 (dd, J = 7.9, 1.4 Hz, 2H), 7.30 -7.23 (m, 1H), 7.20 (d, J =8.1 Hz, 1H), 5.34 (d, J = 7.6 Hz, 1H), 4.44 (dd, J = 13.8, 4.5 Hz, 1H), 4.25 (dt, J = 11.1, 5.9 Hz, 1H), 4.18 (t, J = 12.5 Hz, 1H), 2.46 (s, 3H), 2.16 (d, J= 14.7 Hz, 1H), 2.08 - 1.93 (m, 2H), 1.93 - 1.78 (m, 2H), 1.09 (t, J = 7.4 Hz, 3H). | (method C) 535.4 [M+H]⁺ at 2.83 min |
| 27-1 | (5R*)-5-ethyl-N-[(3S)-9-fluoro-2-oxo-5-phenyl-2,3-dihydro-1H-1,4-benzodiazepin-3-yl]-2-(6-methylpyridin-3-yl)-5H,6H,7H,8H-pyrazolo[3,2-b][1,3]oxazepine-3-carboxamide | F | 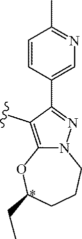 | δ 10.93 (s, 1H), 8.68 (d, J = 2.4 Hz, 1H), 8.55 (d, J = 7.6 Hz, 1H), 7.92 (dd, J = 8.0, 2.3 Hz, 1H), 7.60 (ddd, J = 10.1, 8.2, 1.3 Hz, 1H), 7.57 -7.42 (m, 6H), 7.32 (td, J = 8.0, 4.9 Hz, 1H), 7.22 (d, J = 8.1 Hz, 1H), 7.17 (d, J = 7.9 Hz, 1H), 5.43 (d, J = 7.5 Hz, 1H), 4.44 (dd, J = 13.4, 5.3 Hz, 1H), 4.29 (dt, J = 10.8, 5.8 Hz, 1H), 4.17 (dd, J = 13.9, 10.8 Hz, 1H), 2.47 (s, 3H), 2.16 (d, J = 14.9 Hz, 1H), 2.08 (ddd, J = 13.4, 7.4, 5.2 Hz, 2H), 1.94 (dt, J = 14.0, 6.8 Hz, 1H), 1.88 - 1.77 (m, 1H), 1.06 (t, J = 7.4 Hz, 3H). | (method C) 553.4 [M+H]⁺ at 2.90 min |
| 27-2 | (5S*)-5-ethyl-N-[(3S)-9-fluoro-2-oxo-5-phenyl-2,3-dihydro-1H-1,4-benzodiazepin-3-yl]-2-(6-methylpyridin-3-yl)-5H,6H,7H,8H-pyrazolo[3,2-b][1,3]oxazepine-3-carboxamide | F | 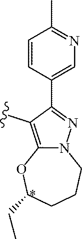 | δ 10.95 (s, 1H), 8.66 (d, J = 2.2 Hz, 1H), 8.51 (d, J = 7.5 Hz, 1H), 7.91 (dd, J = 8.0, 2.3 Hz, 1H), 7.60 (t, J = 9.2 Hz, 1H), 7.56 - 7.42 (m, 6H), 7.32 (td, J = 8.0, 4.9 Hz, 1H), 7.21 (d, J = 8.1 Hz, 1H), 7.17 (d, J = 7.9 Hz, 1H), 5.43 (d, J = 7.4 Hz, 1H), 4.48 - 4.40 (m, 1H), 4.26 (dt, J = 11.4, 6.0 Hz, 1H), 4.18 (t, J = 12.5 Hz, 1H), 2.46 (s, 3H), 2.16 (d, J = 15.0 Hz, 1H), 2.07 (d, J = 12.4 Hz, 1H), 2.04 - 1.95 (m, 2H), 1.92 - 1.78 (m, 2H), 1.09 (t, J = 7.4 Hz, 3H). | (method C) 553.4 [M+H]⁺ at 2.80 min |
| 30-1 | (1S*,8R*)-4-(6-ethylpyridin-3-yl)-N-[(3S)-9-fluoro-2-oxo-5-phenyl-2,3-dihydro-1H-1,4-benzodiazepin-3-yl]-7-oxa-2,3-diazatricyclo[6.2.1. 0^{2,6}]undeca- 3,5-diene-5-carboxamide | F | 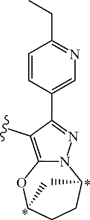 | δ 10.94 (s, 1H), 8.71 - 8.67 (m, 1H), 8.04 (d, J = 7.4 Hz, 1H), 7.94 (dd, J = 8.1, 2.3 Hz, 1H), 7.58 (t, J = 9.3 Hz, 1H), 7.56 -7.42 (m, 5H), 7.32 -7.28 (m, 1H), 7.21 (d, J = 8.1 Hz, 1H), 7.15 (d, J = 8.0 Hz, 1H), 5.48 -5.44 (m, 1H), 5.38 (d, J = 7.0 Hz, 1H), 4.88 - 4.84 (m, 1H), 2.74 (q, J = 7.6 Hz, 2H), 2.46 -2.40 (m, 1H), 2.28 - 2.20 (m, 2H), 2.20 - 2.14 (m, 2H), 2.08 -2.05 (m, 1H), 1.22 (t, J = 8.2, 3H). | (method C) 551.5 [M+H]⁺ at 2.34 |
| 30-2 | (1R*,8S *)-4-(6-ethylpyridin-3-yl)-N-[(3S)-9-fluoro-2-oxo-5-phenyl-2,3-dihydro-1H-1,4-benzodiazepin-3-yl]-7-oxa-2,3-diazatricyclo[6.2.1. 0^{2,6}]undeca- 3,5-diene-5-carboxamide | F | 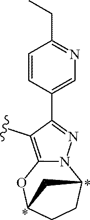 | δ 10.96 (s, 1H), 8.70 - 8.66 (m, 1H), 8.02 (d, J = 7.4 Hz, 1H), 7.93 (dd, J = 8.0, 2.3 Hz, 1H), 7.58 (t, J = 9.2 Hz, 1H), 7.57 -7.50 (m, 1H), 7.53 -7.47 (m, 2H), 7.49 -7.42 (m, 2H), 7.32 -7.28 (m, 1H), 7.21 (d, J = 8.1 Hz, 1H), 7.15 (d, J = 7.9 Hz, 1H), 5.48 - 5.44 (m, 1H), 5.38 (d, J = 7.3 Hz, 1H), 4.87 - 4.83 (m, 1H), 2.74 (q, J = 7.6 Hz, 2H), 2.46 - 2.39 (m, 1H), 2.28 -2.20 (m, 2H), 2.20 - 2.14 (m, | (method C) 551.5 [M+H]⁺ at 2.38 |
| | | | | 1H), 2.11 - 2.03 (m, 1H), 1.22 (t, J = 7.6 Hz, 3H). | |
| 31-1 | (1S*,8R*)-N-[(3S)-9-fluoro-2-oxo-5-phenyl-2,3-dihydro-1H-1,4-benzodiazepin-3-yl]-4-{6-[(propan-2-yl)amino]pyridin-3-yl}-7-oxa-2,3-diazatricyclo[6.2.1. 0^{2,6}]undeca-3,5- diene-5-carboxamide | F | 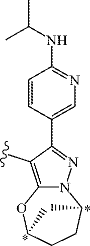 | δ 10.92 (s, 1H), 8.27 - 8.23 (m, 1H), 7.97 (d, J = 7.5 Hz, 1H), 7.65 -7.42 (m, 8H), 7.33 -7.27 (m, 1H), 7.15 (d, J = 7.9 Hz, 1H), 6.41 -6.33 (m, 2H), 5.44 - 5.36 (m, 2H), 4.79 (s, 1H), 4.04 - 3.94 (m, 1H), 2.40 (d, J = 13.0 Hz, 1H), 2.31 -1.99 (m, 4H), 1.13 (d, J = 6.4 Hz, 6H). | (method C) 580.5 [M+H]⁺ at 2.42 min |
| 31-2 | (1R*,8S*)-N-[(3S)-9-fluoro-2-oxo-5-phenyl-2,3-dihydro-1H-1,4-benzodiazepin-3-yl]-4-{6-[(propan-2-yl)amino]pyridin-3-yl}-7-oxa-2,3-diazatricyclo[6.2.1. 0^{2,6}]undeca-3,5- diene-5-carboxamide | F | 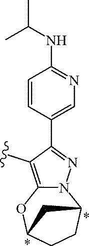 | δ 10.94 (s, 1H), 8.24 (d, J = 2.4 Hz, 1H), 7.96 (d, J = 7.5 Hz, 1H), 7.67 -7.40 (m, 7H), 7.35 -7.25 (m, 1H), 7.15 (d, J= 7.9 Hz, 1H), 6.42 - 6.31 (m, 2H), 5.44 - 5.36 (m, 2H), 4.79 (s, 1H), 4.04 - 3.94 (m, 1H), 2.39 (d, J = 13.1 Hz, 1H), 2.28 -2.12 (m, 4H), 2.10 - 2.00 (m, 1H), 1.13 (d, J = 6.4 Hz, 6H). | (method C) 580.5 [M+H]⁺ at 2.46 min |

### Example 33: Efficacy In Vitro

Compounds were subjected to RSV plaque reduction assays according to the following protocol.

### Plaque Reduction Assay.

Hep-G2 cells (ECACC, 85011430) were passaged in flasks and seeded in 24-well plates in DMEM containing antibiotics and supplemented with 10% FBS. During inoculation and subsequent incubation, cells were cultured in DMEM containing 2% FBS. 100 plaque forming unit/well of RSV (RSV A2 ECACC, 0709161v) was mixed with eight serial dilutions of compound. Subsequently, 100 µL of the virus/compound mixtures was added to confluent Hep-G2 cell monolayers. The cells and virus/compound mixtures were incubated at 37° C. in a humidified 5% CO₂ incubator for 2 h prior to removal of the inoculum and addition of 1 mL of overlay (DMEM containing 2% FBS and 0.8% CMC) containing compound dilutions. The cells and were incubated at 37° C. in a humidified 5% CO₂ incubator for 2 days.

Cells were washed with PBS before adding 75%/25% v/v EtOH/MeOH, for 3 min. Fix ative was removed and plates were washed with PBS. A pre-titrated amount of the primary antibody was added in 200 µL PBS/2% milk powder, and plates incubated for 90 min at 37° C. The plates were washed 3 times with PBS/0.05% Tween20 before addition of rabbit anti-goat horse radish peroxidase in 200 µL PBS/2% milk powder, and incubated for 1 h at 37° C. Following three wash steps with PBS/0.05% Tween20, 200 µL ready-to-use TrueBlue was added and plates were incubated at rt for 10-15 min before washing with water. After removal of water, plates were air-dried in the dark.

Plates were scanned and analysed using the Immunospot S6 Macro analyser, which is equipped with BioSpot analysis software for counting immunostained plaques (virospots). Plaque counts were used to calculate % infection relative to the mean of the plaque count in the virus control wells for RSV. The EC₅₀ value was calculated as 50% reduction in signal, respectively, by interpolation of inhibiti on curves fitted with a 4-parameter nonlinear regression with a variable slope in Dotmatics. Plaque EC₅₀ and cell toxicity CC₅₀ values are a mean of at least two experiments and figures are rounded to whole units.

Results

| Example | RSV A2 Plaque EC₅₀ (nM) | Cell Cytotoxicity CC₅₀ (nM) |
|---|---|---|
| 1 | 79 | >25,000 |
| 2 | 33 | >25,000 |
| 3 | 256 | >25,000 |
| 4 | 219 | >25,000 |
| 5 | 91 | >25,000 |
| 6 | 78 | >25,000 |
| 7 | 52 | >25,000 |
| 8 | 59 | >16,712 |
| 9 | 24 | >25,000 |
| 10 | 31 | >25,000 |
| 11 | 18 | >25,000 |
| 12 | 34 | >25,000 |
| 13 | 34 | >25,000 |
| 14 | 67 | >25,000 |
| 15 | 64 | >25,000 |
| 16 | 64 | >25,000 |
| 17 | 27 | >25,000 |
| 18 | 37 | >25,000 |
| 19 | 37 | >25,000 |
| 20 | 34 | >25,000 |
| 21 | 377 | >25,000 |
| 22 | 103 | >25,000 |
| 23 | 52 | >25,000 |
| 24 | 100 | >25,000 |
| 25 | 83 | >25,000 |
| 26 | 590 | >25,000 |
| 27 | 394 | >25,000 |
| 28 | 45 | >25,000 |
| 29 | 154 | >25,000 |
| 32 | 23 | >25,000 |
| 10-1 | 26 | >25,000 |
| 10-2 | 24 | >25,000 |
| 11-1 | 22 | >25,000 |
| 11-2 | 9 | >25,000 |
| 12-1 | 35 | >25,000 |
| 12-2 | 33 | >25,000 |
| 13-1 | 26 | >25,000 |
| 13-2 | 23 | >25,000 |
| 14-1 | 61 | >25,000 |
| 14-2 | 60 | >25,000 |
| 15-1 | 47 | >25,000 |
| 15-2 | 37 | >25,000 |
| 16-1 | 28 | >25,000 |
| 16-2 | 39 | >25,000 |
| 17-1 | 19 | >25,000 |
| 17-2 | 20 | >25,000 |
| 19-1 | 49 | >25,000 |
| 19-2 | 53 | >25,000 |
| 24-1 | 111 | >25,000 |
| 24-2 | 89 | >25,000 |
| 25-1 | 93 | >25,000 |
| 25-2 | 83 | >25,000 |
| 26-1 | 314 | >25,000 |
| 26-2 | 329 | >25,000 |
| 27-1 | 295 | >25,000 |
| 27-2 | 259 | >25,000 |
| 30-1 | 61 | >25,000 |
| 30-2 | 63 | >25,000 |
| 31-1 | 63 | >25,000 |
| 31-2 | 94 | >25,000 |

### Example 34: Aqueous Formulation

The compound of Example 1 is formulated as a solution in 30% w/v captisol (i.e. sulfobutylether-beta-cyclodextrin) at pH4 according to the following procedure.

A carrier of 30% w/v captisol (i.e. sulfobutylether-beta-cyclodextrin) is prepared by weighing the required amount of captisol into a suitable vessel, adding approximately 80% of the final volume of water and magnetically stirring until a solution is formed. The carrier is then made up to volume with water.

An aqueous solution of a compound of Example 1 is prepared by weighing 175 mg of the compound into a suitable vessel and adding approximately 80% of the required volume of the carrier. Using an aqueous solution of hydrochloric acid, the pH is adjusted to pH2 and the resulting mixture is magnetically stirred until a solution is formed. The formulation is then made up to volume with carrier and the pH is adjusted to pH4 using an aqueous solution of sodium hydroxide.

### Example 35: Tablet Composition

Tablets, each weighing 0.15 g and containing 25 mg of a compound of the invention are manufactured as follows:
Composition for 10,000 tablets
Compound of the invention (250 g)
Lactose (800 g)
Corn starch (415 g)
Talc powder (30 g)
Magnesium stearate (5 g)

The compound of the invention, lactose and half of the corn starch are mixed. The mixture is then forced through a sieve 0.5 mm mesh size. Corn starch (10 g) is suspended in warm water (90 mL). The resulting paste is used to granulate the powder. The granulate is dried and broken up into small fragments on a sieve of 1.4 mm mesh size. The remaining quantity of starch, talc and magnesium is added, carefully mixed and processed into tablets.

Example 36: Injectable Formulation

| | |
|---|---|
| Compound of the invention | 200 mg |
| Hydrochloric Acid Solution 0.1 M or | |
| Sodium Hydroxide Solution 0.1 M q.s. to pH | 4.0 to 7.0 |
| Sterile water q.s. to | 10 mL |

The compound of the invention is dissolved in most of the water (35° C.-40° C.) and the pH adjusted to between 4.0 and 7.0 with the hydrochloric acid or the sodium hydroxide as appropriate. The batch is then made up to volume with water and filtered through a sterile micropore filter into a sterile 10 mL amber glass vial (type 1) and sealed with sterile closures and overseals.

Example 37: Intramuscular Injection

| | |
|---|---|
| Compound of the invention | 200 mg |
| Benzyl Alcohol | 0.10 g |
| Glycofurol 75 | 1.45 g |
| Water for injection q.s to | 3.00 mL |

The compound of the invention is dissolved in the glycofurol. The benzyl alcohol is then added and dissolved, and water added to 3 mL. The mixture is then filtered through a sterile micropore filter and sealed in sterile 3 mL glass vials (type 1).

Example 38: Syrup Formulation

| | |
|---|---|
| Compound of invention | 250 mg |
| Sorbitol Solution | 1.50 g |
| Glycerol | 2.00 g |
| Sodium benzoate | 0.005 g |
| Flavour | 0.0125 mL |
| Purified Water q.s. to | 5.00 mL |

The compound of the invention is dissolved in a mixture of the glycerol and most of the purified water. An aqueous solution of the sodium benzoate is then added to the solution, followed by addition of the sorbital solution and finally the flavour. The volume is made up with purified water and mixed well.

### Example 39: In Vitro Pharmacokinetics

Compounds were subjected to the following assays to investigate liver microsomal stability.

### Microsomal Incubation: Experimental Procedure

Pooled liver microsomes were purchased from a reputable commercial supplier and stored at -80° C. prior to use. Microsomes (final protein concentration 0.5 mg/mL), 0.1 M phosphate buffer pH 7.4 and test compound (final substrate concentration 1 µM; final DMSO concentration 0.25%) were pre-incubated at 37° C. prior to the addition of NADPH (final concentration 1 mM) to initiate the reaction. The final incubation volume was 50 µL. A control incubation was included for each compound tested where 0.1 M phosphate buffer pH 7.4 was added instead of NADPH (minus NADPH). Two control compounds were included with each species. All incubations were performed singularly for each test compound. Each compound was incubated for 0, 5, 15, 30 and 45 min. The control (minus NADPH) was incubated for 45 min only. The reactions were stopped by transferring incubate into acetonitrile at the appropriate time points, in a 1:3 ratio. The termination plates are centrifuged at 3,000 rpm for 20 min at 4° C. to precipitate the protein. Following protein precipitation, the sample supernatants were combined in cassettes of up to 4 compounds, internal standard added, and samples analysed by LC-MS/MS. From a plot of In peak area ratio (compound peak area/internal standard peak area) against time, the gradient of the line was determined. Subsequently, half-life (t½) and intrinsic clearance (CLᵢₙₜ) were calculated.

Results

| Example | Liver Microsomal Stability t_{½} (min); rat / dog / human |
|---|---|
| 2 | 51.2 / 325 / 363 |
| 19 | 128 / 115 / 73.6 |
| 28 | 195 / 1860 / 669 |

### Example 40: In Vivo Pharmacokinetics

The pharmacokinetics of compounds were studied in vivo in rats at doses of 1 mg/kg (IV) and 10 mg/kg (PO).

### Methods

Male rats [Sprague Dawley (SD)] surgically prepared with a jugular vein cannula were treated with experimental compounds via intravenous administration (IV; n=3; 1 mg/kg) or oral administration (PO; n=3; 10 mg/kg). Compounds were formulated as a solution in 40:60 dimethylacetamide:saline (IV administration) and a solution of 10% DMSO, 10% cremaphor in water (80%) (PO administration). Animals were observed for any overt clinical signs or symptoms. Serial blood samples were collected via the cannula at 0.02, 0.08, 0.25, 0.5, 1, 2, 4, 6, 8 and 24 h post IV dosing of compound, and at 0.08, 0.25, 0.5, 1, 2, 4, 6, 8 and 24 h post oral dosing of compound, and plasma was prepared by centrifugation and stored immediately at -80° C. Samples were subsequently thawed, prepared for analysis by protein precipitation with acetonitrile, and analysed by tandem LCMS using electrospray ionisation using a matrix-matched calibration curve. PK parameters were calculated from the resulting data.

Results

| | |
|---|---|
| Example | 19 |
| PO AUCₗₐₛₜ (hr*ng/mL) | 4487 |
| Cl (mL/min/kg) | 16.1 |
| V_{d} (L/kg) | 2.8 |
| Cₘₐₓ (ng/mL) | 949 |
| C 8 h (ng/mL) | 110 |
| IV t_{½} (h) | 2.30 |
| PO t_{½} (h) | 2.9 |
| F (%) | 39.3% |

We claim:

1. A compound which is a benzodiazepine derivative of formula (Ib): 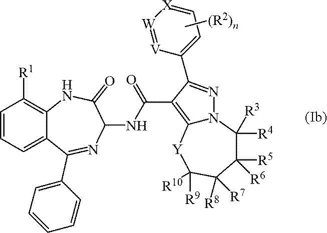
wherein:
R¹ is H or halo;
Y is selected from O, S, SO, SO₂ and NR;
one or two of V, W and X is N or CH and the other one or two is or are CH;
R² is a group selected from C₁-C₆ alkyl, C₁-C₆ hydroxyalkyl, C₁-C₆ haloalkyl, halo, —OR, —NHR″,
—SOₘNR₂, —SOₘR, nitro, —CO₂R, —CN, —CONR₂, —NHCOR and —NR¹¹R¹²;
each R is independently H or C₁-C₆ alkyl;
R¹¹ and R¹² are each independently H or C₁-C₆ alkyl; or R¹¹ and R¹² form, together with the N atom to which they are attached, either (a) a morpholine ring which is optionally bridged by a —CH₂— group linking two ring carbon atoms that are positioned para to each other, or (b) a spiro group of the following formula (b): 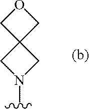
R″ is C₃-C₆ cycloalkyl;
m is 1 or 2;
n is 0, 1 or 2; and
each of R³ to R¹⁰ is independently selected from H, C₁-C₆ alkyl, halo, —OR, —NR₂,
—NHR″,—SOₘNR₂, —SOₘR, nitro, —CO₂R, —CN, —CONR, —NHCOR, —NR¹³R¹⁴ wherein R¹³ and R¹⁴ form, together with the N atom to which they are attached, a morpholine ring, and the following options (i) to (iii):
(i) any two of R³ to R¹⁰ that bond to the same carbon atom form a C₃-C₆ spiro ring;
(ii) any two of R³ to R¹⁰ that bond to non-adjacent carbon atoms form a C₁-C₃ bridgehead group linking the carbon atoms to which they are bonded; and
(iii) any two of R³ to R¹⁰ that bond to adjacent carbon atoms form, together with the carbon atoms to which they are bonded, a C₃-C₆ cycloalkyl group;
and wherein each alkyl group or moiety recited above is linear or branched;
or a pharmaceutically acceptable salt thereof.

2. A compound according to claim 1 wherein, in formula (Ib):
R¹ is H or F;
Y is O, S or SO₂,
each of V and X is CH and W is N or CH;
R² is selected from C₁-C₆ alkyl, halo, —NR₂ and —NHR″, in which R and R″ are as defined in claim 1;
n is 0, 1 or 2; and
each of R³ to R¹⁰ is independently selected from H, C₁-C₆ alkyl, halo and —NR¹³R¹⁴ wherein R¹³ and R¹⁴ form, together with the N atom to which they are attached, a morpholine ring; or
any two of R³ to R¹⁰ that bond to the same carbon atom form a C₃-C₆ spiro ring and the rest of R³ to R¹⁰ are H; or any two of R³ to R¹⁰ that bond to non-adjacent carbon atoms form a C₁-C₃ bridgehead group linking the carbon atoms to which they are bonded and the rest of R³ to R¹⁰ are H.

3. A compound according to claim 1 which is a benzodiazepine derivative of the following formula (I′): 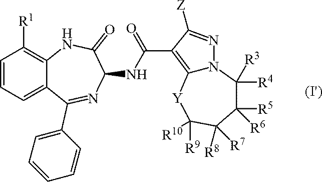
wherein each of R¹, Y and R³ to R¹⁰ is as defined in claim 1 or 2 and Z is selected from the following structures: 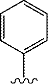 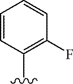 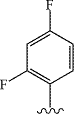 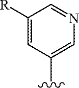 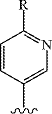 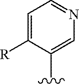 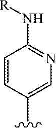 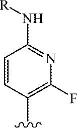 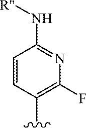 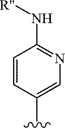 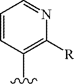
in which R and R″ are as defined in claim 1;
or a pharmaceutically acceptable salt thereof.

4. A compound according to claim 1 wherein R³ to R¹⁰ take the following values:
each of R³ to R¹⁰ is H; or
one or two of R³ to R¹⁰ are C₁-C₃ alkyl, halo or —NR¹³R¹⁴ wherein R¹³ and R¹⁴ form, together with the N atom to which they are attached, a morpholine ring, and the rest of R³ to R¹⁰ are H; or
R³ and R¹⁰ form a C₁ or C₂ bridgehead group linking the carbon atoms to which they are attached, and each of R⁴ to R⁹ is H.

5. A compound according to claim 1 wherein R³ to R¹⁰ take the following values:
each of R³ to R¹⁰ is H; or
one of R³ and R¹⁰ is C₁-C₃ alkyl and the rest of R³ to R¹⁰ are H; or
each of R³ to R⁸ is H and each of R⁹ and R¹⁰ is C₁-C₃ alkyl; or
each of R⁴ to R⁹ is H and each of R³ and R¹⁰ is C₁-C₃ alkyl; or
each of R³ to R⁶, R⁹ and R¹⁰ is H and each of R⁷ and R⁸ is halo; or
one of R⁷ and R⁸ is —NR¹³R¹⁴ wherein R¹³ and R¹⁴ form, together with the N atom to which they are attached, a morpholine ring, and the rest of R³ to R¹⁰ are H; or
R³ and R¹⁰ form a C₁ or C₂ bridgehead group linking the carbon atoms to which they are attached, and each of R⁴ to R⁹ is H.

6. A compound according to claim 1 wherein Y is O.

7. A compound according to claim 1 wherein R¹ is H or F.

8. A compound according to claim 1 which is selected from:
N-[(3S)-2-oxo-5-phenyl-1,3-dihydro-1,4-benzodiazepin-3-yl]-2-phenyl-5,6,7,8-tetrahydropyrazolo[5,1-b][1,3]oxazepine-3-carboxamide;
N-[(3S)-9-Fluoro-2-oxo-5-phenyl-1,3-dihydro-1,4-benzodiazepin-3-yl]-2-(2-fluorophenyl)-5,6,7,8-tetrahydropyrazolo[5,1-b][1,3]oxazepine-3-carboxamide;
2-(5-Methylpyridin-3-yl)-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
N-[(3S)-9-Fluoro-2-oxo-5-phenyl-1,3-dihydro-1,4-benzodiazepin-3-yl]-2-(5-methylpyridin-3-yl)-5,6,7,8-tetrahydropyrazolo[5,1-b][1,3]oxazepine-3-carboxamide;
2-(2-Fluorophenyl)-5,5-dimethyl-N-[(3S)-2-oxo-5-phenyl-1,3-dihydro-1,4-benzodiazepin-3-yl]-7,8-dihydro-6H-pyrazolo[5,1-b][1,3]oxazepine-3-carboxamide;
N-[(3S)-9-Fluoro-2-oxo-5-phenyl-1,3-dihydro-1,4-benzodiazepin-3-yl]-2-(2-fluorophenyl)-5,5-dimethyl-7,8-dihydro-6H-pyrazolo[5,1-b][1,3]oxazepine-3-carboxamide;
2-(2-Fluorophenyl)-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
N-[(3S)-9-Fluoro-2-oxo-5-phenyl-1,3-dihydro-1,4-benzodiazepin-3-yl]-2-phenyl-5,6,7,8-tetrahydropyrazolo[5,1-b][1,3]oxazepine-3-carboxamide;
2-[6-(Cyclopropylamino)-2-fluoropyridin-3-yl]-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
2-[6-(Ethylamino)-2-fluoropyridin-3-yl]-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
2-[6-(Ethylamino)-2-fluoropyridin-3-yl]-N-[(3S)-9-fluoro-2-oxo-5-phenyl-1,3-dihydro-1,4-benzodiazepin-3-yl]-5-methyl-5,6,7,8-tetrahydropyrazolo[5,1-b][1,3]oxazepine-3-carboxamide;
2-[2-Fluoro-6-(propan-2-ylamino)pyridin-3-yl]-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
N-[(3S)-9-fluoro-2-oxo-5-phenyl-1,3-dihydro-1,4-benzodiazepin-3-yl]-2-[2-fluoro-6-(propan-2-ylamino)pyridin-3-yl]-5-methyl-5,6,7,8-tetrahydropyrazolo[5,1-b][1,3]oxazepine-3-carboxamide;
2-(2-Fluorophenyl)-8-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
N-[(3S)-9-fluoro-2-oxo-5-phenyl-1,3-dihydro-1,4-benzodiazepin-3-yl]-2-(2-fluorophenyl)-8-methyl-5,6,7,8-tetrahydropyrazolo[5,1-b][1,3]oxazepine-3-carboxamide;
2-(2,4-Difluorophenyl)-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
2-(2,4-Difluorophenyl)-N-[(3S)-9-fluoro-2-oxo-5-phenyl-1,3-dihydro-1,4-benzodiazepin-3-yl]-5-methyl-5,6,7,8-tetrahydropyrazolo[5,1-b][1,3]oxazepine-3-carboxamide;
2-(2-Fluorophenyl)-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
N-((S)-9-fluoro-2-oxo-5-phenyl-2,3-dihydro-1H-benzo[b]azepin-3-yl)-2-(2-fluorophenyl)-5,6,7,8-tetrahydro-5,8-methanopyrazolo[5,1-b][1,3]oxazepine-3-carboxamide;
N-[(3S)-9-fluoro-2-oxo-5-phenyl-2,3-dihydro-1H-1,4-benzodiazepin-3-yl]-2-(2-fluorophenyl)-5 H,6H,7H,8H-pyrazolo[3,2-b][1,3]thiazepine-3-carboxamide;
N-[(3S)-9-fluoro-2-oxo-5-phenyl-2,3-dihydro-1H-1,4-benzodiazepin-3-yl]-2-(2-fluorophenyl)-4, 4-dioxo-5H,6H,7H,8H-4λ⁶-pyrazolo[3,2-b][1,3]thiazepine-3-carboxamide;
2-(6-Ethylpyridin-3-yl)-6,6-difluoro-N-[(3S)-9-fluoro-2-oxo-5-phenyl-2,3-dihydro-1H-1,4-benzo diazepin-3-yl]-5H,6H,7H,8H-pyrazolo[3,2-b][1,3]oxazepine-3-carboxamide;
6,6-Difluoro-N-[(3S)-9-fluoro-2-oxo-5-phenyl-2,3-dihydro-1H-1,4-benzodiazepin-3-yl]-2-{6-[(p ropan-2-yl)amino]pyridin-3-yl}-5H,6H,7H,8H-pyrazolo[3,2-b][1,3]oxazepine-3-carboxamide;
5-Ethyl-2-(2-fluorophenyl)-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
5-Ethyl-N-[(3S)-9-fluoro-2-oxo-5-phenyl-1,3-dihydro-1,4-benzodiazepin-3-yl]-2-(2-fluorophenyl)-5,6,7,8-tetrahydropyrazolo[5,1-b][1,3]oxazepine-3-carboxamide;
5-Ethyl-2-(6-methylpyridin-3-yl)-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
5-Ethyl-N-[(3S)-9-fluoro-2-oxo-5-phenyl-1,3-dihydro-1,4-benzodiazepin-3-yl]-2-(6-methylpyridin-3-yl)-5,6,7,8-tetrahydropyrazolo[5,1-b][1,3]oxazepine-3-carboxamide;
4-(6-Ethylpyridin-3-yl)-N-[(3S)-9-fluoro-2-oxo-5-phenyl-2,3-dihydro-1H-1,4-benzodiazepin-3-y 1]-7-oxa-2,3-diazatricyclo[6.2.1.0^{2,6}]undeca-3,5-diene-5-carboxamide;
N-[(3S)-9-fluoro-2-oxo-5-phenyl-2,3-dihydro-1H-1,4-benzodiazepin-3-yl]-4-{6-(propan-2-yl)am ino]pyridin-3-yl}-7-oxa-2,3-diazatricyclo[6.2.1.0^{2,6}]undeca-3,5-diene-5-carboxamide;
6,6-Difluoro-N-[(3S)-9-fluoro-2-oxo-5-phenyl-2,3-dihydro-1H-1,4-benzodiazepin-3-yl]-2-(2-flu orophenyl)-5H,6H,7H,8H-pyrazolo[3,2-b][1,3]oxazepine-3-carboxamide;
2-(2-Fluorophenyl)-6-morpholin-4-yl-N-[(3R)-9-fluoro-2-oxo-5-phenyl-1,3-dihydro-1,4-benzodiazepin-3-yl]-5,6,7,8-tetrahydropyrazolo[5,1-b][1,3]oxazepine-3-carboxamide;
4-(2-Fluoro-4-methylsulfonylphenyl)-N-[(3S)-9-fluoro-2-oxo-5-phenyl-1,3-dihydro-1,4-benzodiazepin-3-yl]-7-oxa-2,3-diazatricyclo[6.2.1.02,6]undeca-3,5-diene-5-carboxamide;
(5R*)-2-[6-(ethylamino)-2-fluoropyridin-3-yl]-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
(5S*)-2-[6-(ethylamino)-2-fluoropyridin-3-yl]-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
(5R)-2-[6-(ethylamino)-2-fluoropyridin-3-yl]-N-[(3S)-9-fluoro-2-oxo-5-phenyl-1,3-dihydro-1,4-benzodiazepin-3-yl]-5-methyl-5,6,7,8-tetrahydropyrazolo[5,1-b][1,3]oxazepine-3-carboxamide;
(5S*)-2-[6-(ethylamino)-2-fluoropyridin-3-yl]-N-[(3S)-9-fluoro-2-oxo-5-phenyl-1,3-dihydro-1,4-benzodiazepin-3-yl]-5-methyl-5,6,7,8-tetrahydropyrazolo[5,1-b][1,3]oxazepine-3-carboxamide;
(5R*)-2-[2-fluoro-6-(propan-2-ylamino)pyridin-3-yl]-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
(5S*)-2-[2-fluoro-6-(propan-2-ylamino)pyridin-3-yl]-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
(5R*)-N-[(3S)-9-fluoro-2-oxo-5-phenyl-1,3-dihydro-1,4-benzodiazepin-3-yl]-2-[2-fluoro-6-(propan-2-ylamino)pyridin-3-yl]-5-methyl-5,6,7,8-tetrahydropyrazolo[5,1-b][1,3]oxazepine-3-carboxamide;
(5S*)-N-[(3S)-9-fluoro-2-oxo-5-phenyl-1,3-dihydro-1,4-benzodiazepin-3-yl]-2-[2-fluoro-6-(propan-2-ylamino)pyridin-3-yl]-5-methyl-5,6,7,8-tetrahydropyrazolo[5,1-b][1,3]oxazepine-3-carboxamide;
(8R*)-2-(2-Fluorophenyl)-8-methyl-N-(2-oxo-5-phenyl-1,3-dihydro-1,4-benzodiazepin-3-yl)-5,6,7,8-tetrahydropyrazolo[5,1-b][1,3]oxazepine-3-carboxamide;
(8S*)-2-(2-Fluorophenyl)-8-methyl-N-(2-oxo-5-phenyl-1,3-dihydro-1,4-benzodiazepin-3-yl)-5,6,7,8-tetrahydropyrazolo[5,1-b][1,3]oxazepine-3-carboxamide;
(8R*)-N-(9-fluoro-2-oxo-5-phenyl-1,3-dihydro-1,4-benzodiazepin-3-yl)-2-(2-fluorophenyl)-8-methyl-5,6,7,8-tetrahydropyrazolo[5,1-b][1,3]oxazepine-3-carboxamide;
(8S*)-N-(9-fluoro-2-oxo-5-phenyl-1,3-dihydro-1,4-benzodiazepin-3-yl)-2-(2-fluorophenyl)-8-methyl-5,6,7,8-tetrahydropyrazolo[5,1-b][1,3]oxazepine-3-carboxamide;
(5R*)-2-(2,4-difluorophenyl)-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
(5S*)-2-(2,4-difluorophenyl)-5-methyl-N-[(3S)-2-oxo-5-phenyl-1,3-dihydro-1,4-benzodiazepin-3-yl]-5,6,7,8-tetrahydropyrazolo[5,1-b][1,3]oxazepine-3-carboxamide;
(5R*)-2-(2,4-difluorophenyl)-N-[(3S)-9-fluoro-2-oxo-5-phenyl-1,3-dihydro-1,4-benzodiazepin-3-yl]-5-methyl-5,6,7,8-tetrahydropyrazolo[5,1-b][1,3]oxazepine-3-carboxamide;
(5S*)-2-(2,4-difluorophenyl)-N-[(3S)-9-fluoro-2-oxo-5-phenyl-1,3-dihydro-1,4-benzodiazepin-3-yl]-5-methyl-5,6,7,8-tetrahydropyrazolo[5,1-b][1,3]oxazepine-3-carboxamide;
(1S*,8R*)-N-[(3S)-9-fluoro-2-oxo-5-phenyl-2,3-dihydro-1H-1,4-benzodiazepin-3-yl]-4-(2-fluor ophenyl)-7-oxa-2,3-diazatricyclo[6.2.1.0^{2,6}]undeca-3,5-diene-5-carboxamide;
(1R*,8S*)-N-[(3S)-9-fluoro-2-oxo-5-phenyl-2,3-dihydro-1H-1,4-benzodiazepin-3-yl]-4-(2-fluor ophenyl)-7-oxa-2,3-diazatricyclo[6.2.1.0^{2,6}]undeca-3,5-diene-5-carboxamide;
(5R*)-5-ethyl-2-(2-fluorophenyl)-N-[(3S)-2-oxo-5-phenyl-2,3-dihydro-1H-1,4-benzodiazepin-3-yl]-5H,6H,7H,8H-pyrazolo[3,2-b][1,3]oxazepine-3-carboxamide;
(5S*)-5-ethyl-2-(2-fluorophenyl)-N-[(3S)-2-oxo-5-phenyl-2,3-dihydro-1H-1,4-benzodiazepin-3-yl]-5H,6H,7H,8H-pyrazolo[3,2-b][1,3]oxazepine-3-carboxamide;
(5R*)-5-ethyl-2-(2-fluorophenyl)-N-[(3S)-9-fluoro-2-oxo-5-phenyl-1,3-dihydro-1,4-benzodiazepin-3-yl]-5,6,7,8-tetrahydropyrazolo[5,1-b][1,3]oxazepine-3-carboxamide;
(5S*)-5-ethyl-2-(2-fluorophenyl)-N-[(3S)-9-fluoro-2-oxo-5-phenyl-1,3-dihydro-1,4-benzodiazepin-3-yl]-5,6,7,8-tetrahydropyrazolo[5,1-b][1,3]oxazepine-3-carboxamide;
(SR*)-5-ethyl-2-(6-methylpyridin-3-yl)-N-[(3S)-2-oxo-5-phenyl-2,3-dihydro-1H-1,4-benzodiaze pin-3-yl]-5H,6H,7H,8H-pyrazolo[3,2-b][1,3]oxazepine-3-carboxamide;
(5S*)-5-ethyl-2-(6-methylpyridin-3-yl)-N-[(3S)-2-oxo-5-phenyl-2,3-dihydro-1H-1,4-benzodiaze pin-3-yl]-5H,6H,7H,8H-pyrazolo[3,2-b][1,3]oxazepine-3-carboxamide;
(5R*)-5-ethyl-N-[(3S)-9-fluoro-2-oxo-5-phenyl-2,3-dihydro-1H-1,4-benzodiazepin-3-yl]-2-(6-m ethylpyridin-3-yl)-5H,6H,7H,8H-pyrazolo[3,2-b][1,3]oxazepine-3-carboxamide;
(5S*)-5-ethyl-N-[(3S)-9-fluoro-2-oxo-5-phenyl-2,3-dihydro-1H-1,4-benzodiazepin-3-yl]-2-(6-m ethylpyridin-3-yl)-5H,6H,7H,8H-pyrazolo[3,2-b][1,3]oxazepine-3-carboxamide;
(1S*,8R*)-4-(6-ethylpyridin-3-yl)-N-[(3S)-9-fluoro-2-oxo-5-phenyl-2,3-dihydro-1H-1,4-benzodi azepin-3-yl]-7-oxa-2,3-diazatricyclo[6.2.1.0^{2,6}]undeca-3,5-diene-5-carboxamide;
(1R*,8S*)-4-(6-ethylpyridin-3-yl)-N-[(3S)-9-fluoro-2-oxo-5-phenyl-2,3-dihydro-1H-1,4-benzodi azepin-3-yl]-7-oxa-2,3-diazatricyclo[6.2.1.0^{2,6}]undeca-3,5-diene-5-carboxamide;
(1S*,8R*)-N-[(3S)-9-fluoro-2-oxo-5-phenyl-2,3-dihydro-1H-1,4-benzodiazepin-3-yl]-4-{6-[(pro pan-2-yl)amino]pyridin-3-yl}-7-oxa-2,3-diazatricyclo[6.2.1.0^{2,6}]undeca-3,5-diene-5-carboxamid e;
(1R*,8S*)-N-[(3S)-9-fluoro-2-oxo-5-phenyl-2,3-dihydro-1H-1,4-benzodiazepin-3-yl]-4-{6-[(pro pan-2-yl)amino]pyridin-3-yl}-7-oxa-2,3-diazatricyclo[6.2.1.0^{2,6}]undeca-3,5-diene-5-carboxamid e;
and the pharmaceutically acceptable salts thereof.

9. A pharmaceutical composition which comprises a compound as defined in claim 1 and a pharmaceutically acceptable carrier or diluent.

10-12. (canceled)

13. A method fortreating a subject suffering from or susceptible to an RSV infection, which method comprises administering to said subject an effective amount of a compound as defined in claim 1.

14-15. (canceled)

16. A method according to claim 13, which method further comprises administering to the subject a further therapeutic agent selected from the group consisting of:
(i) a RSV nucleocapsid (N)-protein inhibitor;
(ii) a protein inhibitor, such as one that inhibits the phosphoprotein (P) protein and/or large (L) protein;
(iii) an anti-RSV monoclonal antibody, such as an F-protein antibody;
(iv) an immunomodulating toll-like receptor compound;
(v) a respiratory virus anti-viral, such as an anti-influenza and/or anti-rhinovirus compound; and/or
(vi) an anti-inflammatory compound;
wherein the compound as defined in claim 1 and the further therapeutic agent are administered simultaneously, separately or sequentially.
